SEQUENCE LISTING (50 sequences)

SEQ ID NO: 1 - protein, 1250 aa, Homo sapiens
  misc_feature: Incyte ID No 2780338CD1
MKPPQQSLYLLVDSVDEGCNITEGEQTSTSLSGTVAALLAGHHEFFPPWLLLLCSARKQSKAVTKMFTGFRKISLDDLRKAYIVKDVQQYILHRLDQEEALRQHLTKETAEMLNQLHIKSSGCFLYLERVLDGVVENFIMLREIRDIPGTLNGLYLWLCQRLFVRKQFAKVQPILNVILAACRPLTITELYHAVWTKNMSLTLEDFQRKLDILSKLLVDGLGNTKILFHYSFAEWLLDVKHCTQKYLCNAAEGHRMLAMSYTCQAKNLTPLEAQEFALHLINSNLQLETAELALWMIWNGTPVRDSLSTLIPKEQEVLQLLVKAGAHVNSEDDRTSCIVRQALEREDSIRTLLDNGASVNQCDSNGRTLLANAAYSGSLDVVNLLVSRGADLEIEDAHGHTPLTLAARQGHTKVVNCLIGCGANINHTDQDGWTALRSAAWGGHTEVVSALLYAGVKVDCADADSRTALRAAAWGGHEDIVLNLLQHGAEVNKADNEGRTALIAAAYMGHREIVEHLLDHGAEVNHEDVDGRTALSVAALCVPASKGHASVVSLLIDRGAEVDHCDKDGMTPLLVAAYEGHVDVVDLLLEGGADVDHTDNNGRTPLLAAASMGHASVVNTLLFWGAAVDSIDSEGRTVLSIASAQGNVEVVRTLLDRGLDENHRDDAGWTPLHMAAFEGHRLICEALIEQGARTNEIDNDGRIPFILASQEGHYDCVQILLENKSNIDQRGYDGRNALRVAALEGHRDIVELLFSHGADVNCKDADGRPTLYILALENQLTMAEYFLENGANVEASDAEGRTALHVSCWQGHMEMVQVLIAYHADVNAADNEKRSALQSAAWQGHVKVVQLLIEHGAVVDHTCNQGATALCIAAQEGHIDVVQVLLEHGADPNHADQFGRTAMRVAAKNGHSQIIKLLEKYGASSLNGCSPSPVHTMEQKPLQSLSSKVQSLTIKSNSSGSTGGGDMQPSLRGLPNGPTHAFSSPSESPDSTVDRQKSSLSNNSLKSSKNSSLRTTSSTATAQTVPIDSFHNLSFTEQIQQHSLPRSRSRQSIVSPSSTTQSLGQSHNSPSSEFEWSQVKPSLKSTKASKGGKSENSAKSGSAGKKAKQSNSSQPKVLEYEMTQFDRRGPIAKSGTAAPPKQMPAESQCKIMIPSAQQEIGRSQQQFLIHQQSGEQKKRNGIMTNPNYHLQSNQVFLGRVSVPRTMQDRGHQEVLEGYPSSETELSLKQALKLQIEGSDPSFNYKKETPL

SEQ ID NO: 2 - protein, 2542 aa, Homo sapiens
  misc_feature: Incyte ID No 2317440CD1
MVALSLKICVRHCNVVKTMQFEPSTAVYDACRVIRERVPEAQTGQASDYGLFLSDEDPRKGIWLEAGRTLDYYMLRNGDILEYKKKQRPQKIRMLDGSVKTVMVDDSKTVGELLVTICSRIGITNYEEYSLIQETIEEKKEEGTGTLKKDRTLLRDERKMEKLKAKLHTDDDVNWLDHSRTFREQGVDENETLLLRRKFFYSDQNVDSRDPVQLNLLYVQARDDILNGSHPVSFEKACEFGGFQAQIQFGPHVEHKHKPGFLSLKEFLPKEYIKQRGAEKRIFQEHKNCGEMSEIEAKVKYVKLARSLRTTDSWYLLFQEKMKGKNKLVPRLLGITKDSVMRVDEKTKEVLQEWPLTTVKRWAASPKSFTLDFGEYQESYYSVQTTEGEQISQLIAGYIDIILKKKQSKDRFGLEGDEESTMLEESVSPKKSTILQQQFNRTGKAEHGSVALPAVMRSGSSGPETFNVGSMPSPQQQVMVGQMHRGHMPPLTSAQQALMGTINTSMHAVQQAQDDLSELDSLPPLGQDMASRVWVQNKVDESKHEIHSQVDAITAGTASVVNLTAGDPADTDYTAVGCAITTISSNLTEMSKGVKLLAALMDDEVGSGEDLLRAARTLAGAVSDLLKAVQPTSGEPRQTVLTAAGSIGQASGDLLRQIGENETDERFQDVLMSLAKAVANAAAMLVLKAKNVAQVAEDTVLQNRVIAAATQCALSTSQLVACAKVVSPTISSPVCQEQLIEAGKLVDRSVENCVRACQAATTDSELLKQVSAAASVVSQALHDLLQHVRQFASRGEPIGRYDQATDTIMCVTESIFSSMGDAGEMVRQARVLAQATSDLVNAMRSDAEAEIDMENSKKLLAAAKLLADSTARMVEAAKGAAANPENEDQQQRLREAAEGLRVATNAAAQNAIKKKIVNRLEVAAKQAAAAATQTIAASQNAAVSNKNPAAQQQLVQSCKAVADHIPQLVQGVRGSQAQAEDLSAQLALIISSQNFLQPGSKMVSSAKAAVPTVSDQAAAMQLSQCAKNLATSLAELRTASQKAHEACGPMEIDSALNTVQTLKNELQDAKMAAVESQLKPLPGETLEKCAQDLGSTSKAVGSSMAQLLTCAAQGNEHYTGVAARETAQALKTLAQAARGVAASTTDPAAAHAMLDSARDVMEGSAMLIQEAKQALIAPGDAERQQRLAQVAKAVSHSLNNCVNCLPGQKDVDVALKSIGESSKKLLVDSLPPSTKPFQEAQSELNQAAADLNQSAGEVVHATRGQSGELAAASGKFSDDFDEFLDAGIEMAGQAQTKEDQIQVIGNLKNISMASSKLLLAAKSLSVDPGAPNAKNLLAAAARAVTESINQLITLCTQQAPGQKECDNALRELETVKGMLDNPNEPVSDLSYFDCIESVMENSKVLGESMAGISQNAKTGDLPAFGECVGIASKALCGLTEAAAQAAYLVGISDPNSQAGHQGLVDPIQFARANQAIQMACQNLVDPGSSPSQVLSAATIVAKHTSALCNACRIASSKTANPVAKRHFVQSAKEVANSTANLVKTIKALDGDFSEDNRNKCRIATAPLIEAVENLTAFASNPEFVSIPAQISSEGSQAQEPILVSAKTMLESSSYLIRTARSLAINPKDPPTWSVLAGHSHTVSDSIKSLITSIRDKAPGQRECDYSIDGINRCIRDIEQASLAAVSQSLATRDDISVEALQEQLTSVVQEIGHLIDPIATAARGEAAQLGHKVTQLASYFEPLILAAVGVASKILDHQQQMTVLDQTKTLAESALQMLYAAKEGGGNPKAQHTHDAITEAAQLMKEAVDDIMVTLNEAASEVGLVGGMVDAIAEAMSKLDEGTPPEPKGTFVDYQTTVVKYSKAIAVTAQEMMTKSVTNPEELGGLASQMTSDYGHLAFQGQMAAATAEPEEIGFQIRTRVQDLGHGCIFLVQKAGALQVCPTDSYTKRELIECARAVTEKVSLVLSALQAGNKGTQACITAATAVSGIIADLDTTIMFATAGTLNAENSETFADHRENILKTAKALVEDTKLLVSGAASTPDKLAQAAQSSAATITQLAEVVKLGAASLGSDDPETQVVLINAIKDVAKALSDLISATKGAASKPVDDPSMYQLKGAAKVMVTNVTSLLKTVKAVEDEATRGTRALEATIECIKQELTVFQSKDVPEKTSSPEESIRMTKGITMATAKAVAAGNSCRQEDVIATANLSRKAVSDMLTACKQASFHPDVSDEVRTRALRFGTECTLGYLDLLEHVLVILQKPTPEFKQQLAAFSKRVAGAVTELIQAAEAMKGTEWVDPEDPTVIAETELLGAAASIEAAAKKLEQLKPRAKPKQADETLDFEEQILEAAKSIAAATSALVKSASAAQRELVAQGKVGSIPANAADDGQWSQGLISAARMVAAATSSLCEAANASVQGHASEEKLISSAKQVAASTAQLLVACKVKADQDSEAMRRLQAAGNAVKRASDNLVRAAQKAAFGKADDDDVVVKTKFVGGIAQIIAAQEEMLKKERELEEARKKLAQIRQQQYKFLPTELREDEG

SEQ ID NO: 3 - protein, 560 aa, Homo sapiens
  misc_feature: Incyte ID No 3013470CD1
MSEHSRNSDQEELLDEEINEDEILANLSAEELKELQSEMEVMAPDPSLPVGMIQKDQTDKPPTGNFNHKSLVDYMYWEKASRRMLEEERVPVTFVKSEEKTQEEHEEIEKRNKNMAQYLKEKLNNEIVANKRESKGSSNIQETDEEDEEEEDDDDDDEGEDDGEESEETNREEEGKAKEQIRNCENNCQQVTDKAFKEQRDRPEAQEQSEKKISKLDPKKLALDTSFLKVSTRPSGNQTDLDGSLRRVRKNDPDMKELNLNNIENIPKEMLLDFVNAMKKNKHIKTFSLANVGADENVAFALANMLRENRSITTLNIESNFITGKGIVAIMRCLQFNETLTELRFHNQRHMLGHHAEMEIARLLKANNTLLKMGYHFELPGPRMVVTNLLTRNQDKQRQKRQEEQKQQQLKEQKKLIAMLENGLGLPPGMWELLGGPKPDSRMQEFFQPPPPRPPNPQNVPFSQRSEMMKKPSQAPKYRTDPDSFRVVKLKRIQRKSRMPEAREPPEKTNLKDVIKTLKPVPRNRPPPLVEITPRDQLLNDIRHSSVAYLKPVQLPKELA

SEQ ID NO: 4 - protein, 470 aa, Homo sapiens
  misc_feature: Incyte ID No 1738823CD1
MRTPPALGSQGSEVTGPTFADGELIPREPGFFPEDEEEAMTLAPPEGPQELYTDSPMESTQSLEGSVGSPAEKDGGLGGLFLPEDNAGQTPRKMRHVYNSELLDVYRSQCCKKINLLNDLEARLKNLKANSPNRKISSTAFGRQLMHSSNFSSSNGSTEDLFRDSIDSCDNDITEKVSFLEKKVTELENDSLTNGDLKSKLKQENTQLVHRVHELEEMVKDQETTAEQALEEEARRHREAYGKLEREKATEVELLNARVQQLEEENTELRTTVTRLKSQTEKLDEERQRMSDRLEDTSLRLKDEMDLYKRMMDKLRQNRLEFQKEREATQELIEDLRKELEHLQMYKLDCERPGRGRSASSGLGEFNARAREVELEHEVKRLKQENYKLRDQNDDLNGQILSLSLYEAKNLFAAQTKAQSLAAEIDTASRDELMEALKEQEEINFRLRQYMDKIILAILDHNPSILEIKH

SEQ ID NO: 5 - protein, 898 aa, Homo sapiens
  misc_feature: Incyte ID No 4184551CD1
MSETDHIASTSSDKNVGKTPELKEDSCNLFSGNESSKLENESKLLSLNTDKTLCQPNEHNNRIEAQENYIPDHGGGEDSCAKTDTGSENSEQIANFPSGNFAKHISKTNETEQKVTQILVELRSSTFPESANEKTYSESPYDTDCTKKFISKIKSVSASEDLLEEIESELLSTEFAEHRVPNGMNKGEHALVLFEKCVQDKYLQQEHIIKKLIKENKKHQELFVDICSEKDNLREELKKRTETEKQHMNTIKQLESRIEELNKEVKASRDQLIAQDVTAKNAVQQLHKEMAQRMEQANKKCEEARQEKEAMVMKYVRGEKESLDLRKEKETLEKKLRDANKELEKNTNKIKQLSQEKGRLHQLYETKEGETTRLIREIDKLKEDINSHVIKVKWAQNKLKAEMDSHKETKDKLKETTTKLTQAKEEADQIRKNCQDMIKTYQESEEIKSNELDAKLRVTKGELEKQMQEKSDQLEMHHAKIKELEDLKRTFKEGMDELRTLRTKVKCLEDERLRTEDELSKYKEIINRQKAEIQNLLDKVKTADQLQEQLQRGKQEIENLKEEVESLNSLINDLQKDIEGSRKRESELLLFTERLTSKNAQLQSESNSLQSQFDKVSCSESQLQSQCEQMKQTNINLESRLLKEEELRKEEVQTLQAELACRQTEVKALSTQVEELKDELVTQRRKHASSIKDLTKQLQQARRKLDQVESGSYDKEVSSMGSRSSSSGSLNARSSAEDRSPENTGSSVAVDNFPQVDKAMLIERIVRLQKAHARKNEKIEFMEDHIKQLVEEIRKKTKIIQSYILREESGTLSSEASDFNKVHLSRRGGIMASLYTSHPADNGLTLELSLEINRKLQAVLEDTLLKNITLKENLQTLGTEIERLIKHQHELEQRTKKT

SEQ ID NO: 6 - protein, 817 aa, Homo sapiens
  misc_feature: Incyte ID No 70042484CD1
MMKTEPRGPGGPLRSASPHRSAYEAGIQALKPPDAPGPDEAPKGAHHKKYGSNVHRIKSMFLQMGTTAGPSGEAGGGAGLAEAPRASERGVRLSLPRASSLNENVDHSALLKLGTSVSERVSRFDSKPAPSAQPAPPPHPPSRLQETRKLFERSAPAAAGGDKEAAARRLLRQERAGLQDRKLDVVVRFNGSTEALDKLDADAVSPTVSQLSAVFEKADSRTGLHRGPGLPRAAGVPQVNSKLVSKRSRVFQPPPPPPPAPSGDAPAEKERCPAGQQPPQHRVAPARPPPKPREVRKIKPVEVEESGESEAESAPGEVIQAEVTVHAALENGSTVATAASPAPEEPKAQAAPEKEAAAVAPPERGVGNGRAPDVAPEEVDESKKEDFSEADLVDVSAYSGLGEDSAGSALEEDDEDDEEDGEPPYEPESGCVEIPGLSEEEDPAPSRKIHFSTAPIQVFSTYSNEDYDRRNEDVDPMAASAEYELEKRVERLELFPVELEKDSEGLGISIIGMGAGADMGLEKLGIFVKTVTEGGAAHRDGRIQVNDLLVEVDGTSLVGVTQSFAASVLRNTKGRVRFMIGRERPGEQSEVAQLIQQTLEQERWQREMMEQRYAQYGEDDEETGEYATDEDEELSPTFPGGEMAIEVFELAENEDALSPVDMEPEKLVHKFKELQIKHAVTEAEIQQLKRKLQSLEQEKGRWRVEKAQLEQSVEENKERMEKLEGYWGEAQSLCQAVDEHLRETQAQYQALERKYSKAKRLIKDYQQKEIEFLKKETAQRRVLEESELARKEEMDKLLDKISELEGNLQTLRNSNST

SEQ ID NO: 7 - protein, 664 aa, Homo sapiens
  misc_feature: Incyte ID No 3236274CD1
MPAVDKLLLEEALQDSPQTRSLLSVFEEDAGTLTDYTNQLLQAMQRVYGAQNEMCLATQQLSKQLLAYEKQNFALGKGDEEVISTLHYFSKVVDELNLLHTELAKQLADTMVLPIIQFREKDLTEVSTLKDLFGLASNEHDLSMAKYSRLPKKKENEKVKTEVGKEVAAARRKQHLSSLQYYCALNALQYRKQMAMMEPMIGFAHGQINFFKKGAEMFSKRMDSFLSSVADMVQSIQVELEAEAEKMRVSQQELLSVDESVYTPDSDVAAPQINRNLIQKAGYLNLRNKTGLVTTTWERLYFFTQGGNLMCQPRGAVAGGLIQDLDNCSVMAVDCEDRRYCFQITTPNGKSGIILQAESRKENEEWICAINNISRQIYLTDNPEAVAIKLNQTALQAVTPITSFGKKQESSCPSQNLKNSEMENENDKIVPKATASLPEAEELIAPGTPIQFDIVLPATEFLDQNRGSRRTNPFGETEDESFPEAEDSLLQQMFIVRFLGSMAVKTDSTTEVIYEAMRQVLAARAIHNIFRMTESHLMVTSQSLRLIDPQTQVSRANFELTSVTQFAAHQENKRLVGFVIRVPESTGEESLSTYIFESNSEGEKICYAINLGKEIIEVQKDPEALAQLMLSIPLTNDGKYVLLNDQPDDDDGNPNEHRGAESEA

SEQ ID NO: 8 - protein, 780 aa, Homo sapiens
  misc_feature: Incyte ID No 7179725CD1
MAWPCITRACCIARFWNQLDKADIAVPLVFTKYSEATEHPGAPPQPPPPQQQAQPALAPPSARAVAIETQPAQGELDAVARATGPAPGPTGEREPAAGPGRSGPGPGLGSGSTSGPADSVMRQDYRAWKVQRPEPSCRPRSEYQPSDAPFERETQYQKDFRAWPLPRRGDHPWIPKPVQISAASQASAPILGAPKRRPQSQERWPVQAAAEAREQEAAPGGAGGLAAGKASGADERDTRRKAGPAWMVRRAEGLGHEQTPLPAAQAQVQATGPEAGRGRAAADALNRQIREEVASAVSSSYRNEFRAWTDIKPVKPIKAKPQYKPPDDKMVHETSYSAQFKGEASKPTTADNKVIDRRRIRSLYSEPFKEPPKVEKPSVQSSKPKKTSASHKPTRKAKDKQAVSGQAAKKKSAEGPSTTKPDDKEQSKEMNNKLAEAKESLAQPVSDSSKTQGPVATEPDKDQGSVVPGLLKGQGPMVQEPLKKQGSVVPGPPKDLGPMIPLPVKDQDHTVPEPLKNESPVISAPVKDQGPSVPVPPKNQSPMVPAKVKDQGSVVPESLKDQGPRIPEPVKNQAPMVPAPVKDEGPIVPAPVKDEGPMVSAPIKDQDPMVPEHPKDESAMATAPIKNQGSMVSEPVKNQGLVVSGPVKDQDVVVPEHAKVHDSAVVAPVKNQGPVVPESVKNQDPILPVLVKDQGPTVLQPPKNQGRIVPEPLKNQVPIVPVPLKDQDPLVPVPAKDQGPAVPEPLKTQGPRDPQLPTVSPLPRVMIPTAPHTEYIESSP

SEQ ID NO: 9 - protein, 766 aa, Homo sapiens
  misc_feature: Incyte ID No 1966217CD1
MEEIKPASASCVSKEKPSKVSDLISRFEGGSSLSNYSDLKKESAVNLNAPRTPGRHGLTTTPQQKLLSQHLPQRQGNDTDKTQGAQTCVANGVMAAQNQMECEEEKAATLSSDTSIQASEPLLDTHIVNGERDETATAPASPTTDSCDGNASDSSYRTPGIGPVLPLEERGAETETKVQERENGESPLELEQLDQHHEMKETNEQKLHKIANELLLTERAYVNRLDLLDQVFYCKLLEEANRGSFPAEMVNKIFSNISSINAFHSKFLLPELEKRMQEWETTPRIGDILQKLAPFLKMYGEYVKGFDNAMELVKNMTERIPQFKSVVEEIQKQKICGSLTLQHHMLEPVQRIPRYEMLLKDYLRKLPPDSLDWNDAKKSLEIISTAASHSNSAIRKMENLKKLLEIYEMLGEEEDIVNPSNELIKEGQILKLAARNTSAQERYLFLFNNMLLYCVPKFSLVGSKFTVRTRVGIDGMKIVETQNEEYPHTFQVSGKERTLELQASSAQDKEEWIKALQETIDAFHQRHETFRNAIAKDNDIHSEVSTAELGKRAPRWIRDNEVTMCMKCKEPFNALTRRRHHCRACGYVVCWKCSDYKAQLEYDGGKLSKVCKDCYQIISGFTDSEEKKRKGILEIESAEVSGNSVVCSFLQYMEKSKPWQKAWCVIPKQDPLVLYMYGAPQDVRAQATIPLLGYVVDEMPRSADLPHSFKLTQSKSVHSFAADSEELKQKWLKVILLAVTGETPGGPNEHPATLDDHPEPKKKSEC

SEQ ID NO: 10 - protein, 420 aa, Homo sapiens
  misc_feature: Incyte ID No 1598186CD1
MRPHLGDTIEEPSVLERVLCPRSEGGSRTGRAGPSGWAPPRGARSAESTDRLKGMALTVDVAGPAPWGFRITGGRDFHTPIMVTKVAERGKAKDADLRPGDIIVAINGESAEGMLHAEAQSKIRQSPSPLRLQLDRSQATSPGQTNGDSSLEVLATRFQGSVRTYTESQSSLRSSYSSPTSLSPRAGSPFSPPPSSSSLTGEAAISRSFQSLACSPGLPAADRLSYSGRPGSRQAGLGRAGDSAVLVLPPSPGPRSSRPSMDSEGGSLLLDEDSEVFKMLQENREGRAAPRQSSSFRLLQEALEAEERGGTPAFLPSSLSPQSSLPASRALATPPKLHTCEKCSTSIANQAVRIQEGRYRHPGCYTCADCGLNLKMRGHFWEDACAMEGMRLSLEALEGMVEGAKRRDRRKTRRPIQPSW

SEQ ID NO: 11 - protein, 417 aa, Homo sapiens
  misc_feature: Incyte ID No 7493044CD1
MSFNTCSTFSTCSTFSTNYQSLGRLDSQRVASIYAGAGGSGSQISLSHSTSLQGGMGSRGLSTGMAGGLAGMGGIQNEKETMQSLNDLLASYLDRVRNLETENAGEQYLGAPGEERPQVRDWSHYFKTIEEDLRAIFTNTVDNAHIVLQIDNARLAADDLRVKYETELAMCQSVESDIRELRKVIDYTNVTQLQLEIEIELKEELLFMKKNHEEEVKGVQAQIASSGLTVEVDAPKSQDLAKIMAENWAQYGKLARKNREELDKYWSQQIQKSTTVVTTQLAEVGAAEMLMELRHTVQSLEILDSMRNLKAILENSLREMEARYTLQMEQLNRIRPHLESELAQTQAEGQGQAQEYEALQNIKVKLEAEITTYHRLLEDGEDFNLGDALDSSNSMQTIQKTTTCQIVDGKVVSENEQ

SEQ ID NO: 12 - protein, 567 aa, Homo sapiens
  misc_feature: Incyte ID No 7925017CD1
MQNKLCFSLDPFQLPAKTEPIKERAVQPAPTRKPTVIRIPAKPGSLHEDPQSPPPLPAEKPIGNTFSTVSGKLSNVERTRNLESNHPGQTGGFVRVPPRLPPRPVNGKTIPTQQPPTKVPPERPPPPKLSATRRSNKKLPFNRSSSDMDLQKKQSNLATGLSKAKSQVFKNQDPVLPPRPKPGHPLYSKYMLSVPHGIANEDIVSQNPGELSCKRGDVLVMLKQTENNYLECQKGEDTGRVHLSQMKIITPLDEHLRSRPNDPSHAQKPVDSGAPHAVVLHDFPAEQVDDLNLTSGEIVYLLEKIDTDWYRGNCRNQIGIFPANYVKVIIDIPEGGNGKRECVSSHCVKGSRCVARFEYIGEQKDELSFSEGEIIILKEYVNEEWARGEVRGRTGIFPLNFVEPVEDYPTSGANVLSTKVPLKTKKEDSGSNSQVNSLPAEWCEALHSFTAETSDDLSFKRGDRIQILERLDSDWCRGRLQDREGIFPAVFVRPCPAEAKSMLAIVPKGRKAKALYDFRGENEDELSFKAGDIITELESVDDDWMSGELMGKSGIFPKNYIQFLQIS

SEQ ID NO: 13 - protein, 648 aa, Homo sapiens
  misc_feature: Incyte ID No 6758789CD1
MPRAARAGGCGARWQGQLCVLTRLLCSPLCLTHPPLNAALLSFPTVSQPQAAPSPLEKSPSTAILCNTCGNVCKGEVLRVQDKYFHIKCFVCKACGCDLAEGGFFVRQGEYICTLDYQRLYGTRCFSCDQFIEGEVVSALGKTYHPDCFVCAVCRLPFPPGDRVTFNGKECMCQKCSLPVSVGSSAHLSQGLRSCGGCGTEIKNGQALVALDKHWHLGCFKCKSCGKLLNAEYISKDGLPYCEADYHAKFGIRCDSCEKYITGRVLEAGEKHYHPSCALCVRCGQMFAEGEEMYLQGSSIWHPACRQAARTEDRNKETRTSSESIISVPASSTSGSPSRVIYAKLGGEILDYRDLAALPKSKAIYDIDRPDMISYSPYISHSAGDRQSYGEGDQDDRSYKQCRTSSPSSTGSVSLGRYTPTSRSPQHYSRPAGTVSVGTSSCLSLSQHPSPTSVFRHHYIPYFRGSESGRSTPSLSVLSDSKPPPSTYQQAPRHFHVPDTGVKDNIYRKPPIYRQHAARRSDGEDGSLDQDNRKQKSSWLMLKGDADTRTNSPDLDTQSLSHSSGTDRDPLQRMAGDSFHSQYKIYPYDSLIVTNRIRVKLPKDVDRTRLERHLSPEEFQEVFGMSIEEFDRLALWKRNDLKKKALLF

SEQ ID NO: 14 - protein, 270 aa, Homo sapiens
  misc_feature: Incyte ID No 7488249CD1
MNPQCARCGKVVYPTEKVNCLDKYWHKGCFHCEVCKMALNMNNYKGYEKKPYCNAHYPKQSFTTVADTPENLRLKQQSELQSQVKYKRDFEESKGRGFSIVTDTPELQRLKRTQEQISNVKYHEDFEKTKGRGFTPVVDDPVTERVRKNTQVVSDAAYKGVHPHIVEMDRRPGIIVAPVLPGAYQQSHSQGYGYMHQTSVSSMRSMQHSPNLRTYRAMYDYSAQDEDEVSFRDGDYIVNVQPIDDGWMYGTVQRTGRTGMLPANYIEFVN

SEQ ID NO: 15 - protein, 1893 aa, Homo sapiens [sequence omitted]

SEQ ID NO: 16 - protein, 869 aa, Homo sapiens
  misc_feature: Incyte ID No 931056CD1
MGKKIKKEVEPPPKDVFDPLMIESKKAATVVLMLNSPEEEILAKACEAIYKFALKGEENKTTLLELGAVEPLTKLLTHEDKIVRRNATMIFGILASNNDVKKLLRELDVMNSVIAQLAPEEEVVIHEFASLCLANMSAEYTSKVQIFEHGGLEPLIRLLSSPDPDVKKNSMECIYNLVQDFQCRAKLQELNAIPPILDLLKSEYPVIQLLALKTLGVIANDKESRTMLRDNQGLDHLIKILETKELNDLHIEALAVIANCLEDMDTMVQIQQTGGLKKLLSFAENSTIPDIQKNAAKAITKAAYDPENRKLFHEQEVEKCLVALLGSENDGTKIAASQAISAMCENSGSKDFFNNQGIPQLIQLLKSDNEEVREAAALALANLTTCNPANANAAAEADGIDPLINLLSSKRDGAIANAATVLTNMAMQEPLRLNIQNHDIMHAIISPLRSANTVVQSKAALAVTATACDVEARTELRNSGGLEPLVELLRSKNDEVRKHASWAVMVCAGDELTANELCRLGALDILEEVNVSGTRKNKFSEAAYNKLLNNNLSLKYSQTGYLSSSNIINDGFYDYGRINPGTKLLPLKELCLQEPSDLRAVLLINSKSYVSPPSSMEDKSDVGYGRSISSSSSLRRSSKEKNNYHFSAGFGSPIEDKSEPASGRNTVLSKSATKEKGWRKSKGKKEEEKVKEEEEVMVVPKFVGEGSSDKEWCPPSDPDFSMYVYEVTKSILPITNIKEQIEDLAKYVAEKMGGKIPKEKLPDFSWELHISELKFQLKSNVIPIGHVKKGIFYHRALLFKALADRIGIGCSLVRGEYGRAWNEVMLQNDSRKGVIGGLPAPEMYVIDLMFHPGGLMKLRSREADLYRFI

SEQ ID NO: 17 - protein, 572 aa, Homo sapiens
  misc_feature: Incyte ID No 2578937CD1
MNTSIPYQQNPYNPRGSSNVIQCYRCGDTCKGEVVRVHNNHFHIRCFTCQVCGCGLAQSGFFFKNQEYICTQDYQQLYGTRCDSCRDFITGEVISALGRTYHPKCFVCSLCRKPFPIGDKVTFSGKECVCQTCSQSMASSKPIKIRGPSHCAGCKEEIKHGQSLLALDKQWHVSCFKCQTCSVILTGEYISKDGVPYCESDYHAQFGIKCETCDRYISGRVLEAGGKHYHPTCARCVRCHQMFTEGEEMYLTGSEVWHPICKQAARAEKKLKHRRTSETSISPPGSSIGSPNRVICDIYENLDLRQRRASSPGYIDSPTYSRQGMSPTFSRSPHHYYRSGDLSTATKSKTSEDISQTSKYSPIYSPDPYYASESEYWTYHGSPKVPRARRFSSGGEEDDFDRSMHKLQSGIGRLILKEEMKARSSSYADPWTPPRSSTSSREALHTAGYEMSLNGSPRSHYLADSDPLISKSASLPAYRRNGLHRTPSADLFHYDSMNAVNWGMREYKIYPYELLLVTTRGRNRLPKDVDRTRLERHLSQEEFYQVFGMTISEFDRLALWKRNELKKQARLF

SEQ ID NO: 18 - protein, 1056 aa, Homo sapiens
  misc_feature: Incyte ID No 489786CD1
MAVLKLTDQPPLVQAIFSGDPEEIRMLIHKTEDVNTLDSEKRTPLHVAAFLGDAEIIELLILSGARVNAKDNMWLTPLHRAVASRSEEAVQVLIKHSADVNARDKNWQTPLHVAAANKAVKCAEVIIPLLSSVNVSDRGGRTALHHAALNGHVEMVNLLLAKGANINAFDKKDRRALHWAAYMGHLDVVALLINHGAEVTCKDKKGYTPLHAAASNGQINVVKHLLNLGVEIDEINVYGNTALHIACYNGQDAVVNELIDYGANVNQPNNNGFTPLHFAAASTHGALCLELLVNNGADVNIQSKDGKSPLHMTAVHGRFTRSQTLIQNGGEIDCVDKDGNTPLHVAARYGHELLINTLITSGADTAKCGIHSMFPLHLAALNAHSDCCRKLLSSGFEIDTPDKFGRTCLHAAAAGGNVECIKLLQSSGADFHKKDKCGRTPLHYAAANCHFHCIETLVTTGANVNETDDWGRTALHYAAASDMDRNKTILGNAHDNSEELERARELKEKEATLCLEFLLQNDANPSIRDKEGYNSIHYAAAYGHRQCLELLLERTNSGFEESDSGATKSPLHLAAYNGHHQALEVLLQSLVDLDIRDEKGRTALDLAAFKGHTECVEALINQGASIFVKDNVTKRTPLHASVINGHTLCLRLLLEIADNPEAVDVKDAKGQTPLMLAVAYGHIDAVSLLLEKEANVDTVDILGCTALHRGIMTGHEECVQMLLEQEVSILCKDSRGRTPLHYAAARGHATWLSELLQMALSEEDCCFKDNQGYTPLHWACYNGNENCIEVLLEQKCFRKFIGNPFTPLHCAIINDHGNCASLLLGAIDSSIVSCRDDKGRTPLHAAAFADHVECLQLLLRHSAPVNAVDNSGKTALMMAAENGQAGAVDILVNSAQADLTVKDKDLNTPLHLACSKGHEKCALLILDKIQDESLINEKNNALQTPLHVAARNGLKVLVEELLAKGACVLAVDENGHTPALACAPNKDVADCLALILATMMPFSPSSTMMAVNFVCLKKDNLSRTTLSNLGSMVSLCSNNVGSEDGYNENDSDSETF

SEQ ID NO: 19 - protein, 923 aa, Homo sapiens
  misc_feature: Incyte ID No 2240034CD1
MDGFAGSLDDSISAASTSDVQDRLSALESRVQQQEDEITVLKAALADVLRRLAISEDHVASVKKSVSSKGQPSPRAVIPMSCITNGSGANRKPSHTSAVSIAGKETLSSAAKSIKRPSPAEKSHNSWENSDDSRNKLSKIPSTPKLIPKVTKTADKHKDVIINQEGEYIKMFMRGRPITMFIPSDVDNYDDIRTELPPEKLKLEWAYGYRGKDCRANVYLLPTGEIVYFIASVVVLFNYEERTQRHYLGHTDCVKCLAIHPDKIRIATGQIAGVDKDGRPLQPHVRVWDSVTLSTLQIIGLGTFERGVGCLDFSKADSGVHLCVIDDSNEHMLTVWDWQKKAKGAEIKTTNEVVLAVEFHPTDANTIITCGKSHIFFWTWSGNSLTRKQGIFGKYEKPKFVQCLAFLGNGDVLTGDSGGVMLIWSKTTVEPTPGKGPKGVYQISKQIKAHDGSVFTLCQMRNGMLLTGGGKDRKIILWDHDLNPEREIEVPDQYGTIRAVAEGKADQFLVGTSRNFILRGTFNDGFQIEVQGHTDELWGLATHPFKDLLLTCAQDRQVCLWNSMEHRLEWTRLVDEPGHCADFHPSGTVVAIGTHSGRWFVLDAETRDLVSIHTDGNEQLSVMRYSIDGTFLAVGSHDNFIYLYVVSENGRKYSRYGRCTGHSSYITHLDWSPDNKYIMSNSGDYEILYWDIPNGCKLIRNRSDCKDIDWTTYTCVLGFQVFGVWPEGSDGTDINALVRSHNRKVIAVADDFCKVHLFQYPCSKAKAPSHKYSAHSSHVTNVSFTHNDSHLISTGGKDMSIIQWKLVEKLSLPQNETVADTTLTKAPVSSTESVIQSNTPTPPPSQPLNETAEEESRISSSPTLLENSLEQTVEPSEDHSEEESEEGSGDLGEPLYEEPCNEISKEQAKATLLEDQQDPSPSS

SEQ ID NO: 20 - protein, 201 aa, Homo sapiens
  misc_feature: Incyte ID No 3438037CD1
MSFATLRPAPPGRYLYPEVSPLSEDEDRGSDSSGSDEKPCRVHAARCGLQGARRRAGGRRAGGGGPGGRPGREPRQRHTANARERDRTNSVNTAFTALRTLIPTEPADRKLSKIETLRLASSYISHLGNVLLAGEACGDGQPCHSGPAFFHAARAGSPPPPPPPPPARDGENTQPKQICTFCLSNQRKLSKDRDRKTAIRS

SEQ ID NO: 21 - protein, 793 aa, Homo sapiens
  misc_feature: Incyte ID No 6578021CD1
MDPQPLRGASEEPSGTQSEGGGSSSSGAGSPGPPGILRPLQPPQRADTPRRNSSSSSSPSEWPRQKLSRKAISSANLLVRSGSTESRGGKDPLSSPGGPGSRRSNYNLEGISVKMFLRGRPITMYIPSGIRSLEELPSGPPPETLSLDWVYGYRGRDSRSNLFVLRSGEVVYFIACVVVLYRPGGGPGGPGGGGQRHYRGHTDCVRCLAVHPDGVRVASGQTAGVDKDGKPLQPVVHIWDSETLLKLQEIGLGAFERGVGALAFSAADQGAFLCVVDDSNEHMLSVWDCSRGMKLAEIKSTNDSVLAVGFNPRDSSCIVTSGKSHVHFWNWSGGVGVPGNGTLTRKQGVFGKYKKPKFIPCFVFLPDGDILTGDSEGNILTWGRSPSDSKTPGRGGAKETYGIVAQAHAHEGSIFALCLRRDGTVLSGGGRDRRLVQWGPGLVALQEAEIPEHFGAVRAIAEGLGSELLVGTTKNALLRGDLAQGFSPVIQGHTDELWGLCTHPSQNRFLTCGHDRQLCLWDGESHALAWSIDLKETGLCADFHPSGAVVAVGLNTGRWLVLDTETREIVSDVIDGNEQLSVVRYSPDGLYLAIGSHDNVIYIYSVSSDGAKSSRFGRCMGHSSFITHLDWSKDGNFIMSNSGDYEILYWDVAGGCKQLKNRYESRDREWATYTCVLGFHVYGVWPDGSDGTDINSLCRSHNERVVAVADDFCKVHLFQYPCARAKAPSRMYGGHGSHVTSVRFTHDDSHLVSLGGKDASIFQWRVLGAGGAGPAPATPSRTPSLSPASSLDV

SEQ ID NO: 22 - protein, 1094 aa, Homo sapiens
  misc_feature: Incyte ID No 8013295CD1
MNLRCDLLDKKANPNAKALNGFTPLHIACKKNRIKVMELLLKHGASIQAVTESGLTPIHVAAFMGHVNIVSQLMHHGASPNTTNVRGETALHMAARSGQAEVVRYLVQDGAQVEAKAKDDQTPLHISARLGKADIVQQLLQQGASPNAATTSGYTPLHLSAREGHEDVAAFLLDHGASLSITTKKGFTPLHVAAKYGKLEVANLLLQKSASPDAAGKSGLTPLHVAAHYDNQKVALLLLDQGASPHAAAKNGYTPLHIAAKKNQMDIATTLLEYGADANAVTRQGIASVHLAAQEGHVDMVSLLLGRNANVNLSNKSGLTPLHLAAQEDRVNVAEVLVNQGAHVDAQTKMGYTPLHVGCHYGNIKIVNFLLQHSAKVNAKTKNGYTPLHQAAQQGHTHIINVLLQNNASPNELTVNGNTALGIARRLGYISVVDTLKIVTEETMTTTTVTEKHKMNVPETMNEVLDMSDDEGEDAMTGDTDKYLGPQDLKELGDDSLPAEGYMGFSLGARSASLRSFSSDRSYTLNRSSYARDSMMIEELLVPSKEQHLTFTREFDSDSLRHYSWAADTLDNVNLVSSPIHSGFLVSFMVDARGGSMRGSRHHGMRIIIPPRKCTAPTRITCRLVKRHKLANPPPHGERRGISSRLVEMGPAGAQFLGPVIVEIPHFGSMRGKERELIVLRSENGETWKEHQFDSKNEDLTELLNGMDEELDSPEELGKKRICRIITKDFPQYFAVVSRIKQESNQIGPEGGILSSTTVPLVQASFPEGALTKRIRVGLQAQPVPDEIVKKILGNKATFSPIVTVEPRRRKFHKPITMTIPVPPPSGEGVSNGYKGDTTPNLRLLCSITGGTSPAQWEDITGTTPLTFIKDCVSFTTNVSARFWLADCHQVLETVGLATQLYRELICVPYMAKFVVFAKMNDPVESSLRCFCMTDDKVDKTLEQQENFEEVARSKDIEVLEGKPIYVDCYGNLAPLTKGGQQLVFNFYSFKENRLPFSIKIRDTSQEPCGRLSFLKEPKTTKGLPQTAVCNLNITLPAHKKIEKTDRRQSFASLALRKRYSYLTEPGMSEFPDTSTNPGQCFRRRDIFSMRSKL

SEQ ID NO: 23 - protein, 818 aa, Homo sapiens
  misc_feature: Incyte ID No 5001859CD1
MERYKALEQLLTELDDFLKILDQENLSSTALVKKSCLAELLRLYTKSSSSDEEYIYMNKVTINKQQNAESQGKAPEEQGLLPNGEPSQHSSAPQKSLPDLPPPKMIPERKQLAIPKTESPEGYYEEAEPYDTSLNEDGEAVSSSYESYDEEDGSKGKSAPYQWPSPEAGIELMRDARICAFLWRKKWLGQWAKQLCVIKDNRLLCYKSSKDHSPQLDVNLLGSSVIHKEKQVRKKEHKLKITPMNADVIVLGLQSKDQAEQWLRVIQEVSGLPSEGASEGNQYTPDAQRFNCQKPDIAEKYLSASEYGSSVDGHPEVPETKDVKKKCSAGLKLSNLMNLGRKKSTSLEPVERSLETSSYLNVLVNSQWKSRWCSVRDNHLHFYQDRNRSKVAQQPLSLVGCEVVPDPSPDHLYSFRILHKGEELAKLEAKSSEEMGHWLGLLLSESGSKTDPEEFTYDYVDADRVSCIVSAAKNSLLLMQRKFSEPNTYIDGLPSQDRQEELYDDVDLSELTAAVEPTEEATPVADDPNERESDRVYLDLTPVKSFLHGPSSAQAQASSPTLSCLDNATEALPADSGPGPTPDEPCIKCPENLGEQQLESLEPEDPSLRITTVKIQTEQQRISFPPSCPDAVVATPPGASPPVKDRLRVTSAEIKLGKNRTEAEVKRYTEEKERLEKKKEEIRGHLAQLRKEKRELKETLLKCTDKEVLASLEQKLKEIDEECRGEESRRVDLELSIMEVKDNLKKAEAGPVTLGTTVDTTHLENVSPRPKAVTPASAPDCTPVNSATTLKNRPLSVVVTGKGTVLQKAKEWEKKGAS

SEQ ID NO: 24 - protein, 617 aa, Homo sapiens
  misc_feature: Incyte ID No 7506133CD1
MPAVDKLLLEEALQDSPQTRSLLSVFEEDAGTLTDYTNQLLQAMQRVYGAQNEMCLATQQLSKQLLAYEKQNFALGKGDEEVISTLHYFSKVVDELNLLHTELAKQLADTMVLPIIQFREKDLTEVSTLKDLFGLASNEHDLSMAKYSRLPKKKENEKVKTEVGKEVAAARRKQHLSSLQYYCALNALQYRKQMAMMEPMIGFAHGQINFFKKGAEMFSKRMDSFLSSVADMVQSIQVELEAEAEKMRVSQQELLSVDESVYTPDSDVAAPQINRNLIQKAGYLNLRNKTGLVTTTWERLYFFTQGGNLMCQPRGAVAGGLIQDLDNCSVMAVDCEDRRYCFQITTPNGKSGIILQAESRKENEEWICAINNISRQIYLTDNPEAVAIKLNQTALQAVTPITSFGKKQESSCPSQNLKNSEMENENDKIVPKATASLPEAEELIAPGTPIQFDIVLPATEFLDQNRGSRRTNPFGETEDESFPEAEDSLLQQMFIVRFLGSMAVKTDSTTEVIYEAMRQVLAARAIHNIFRMTESHLMVTSQSLRLIDPQTQVSRANICYAINLGKEIIEVQKDPEALAQLMLSIPLTNDGKYVLLNDQPDDDDGNPNEHRGAESEA

SEQ ID NO: 25 - protein, 402 aa, Homo sapiens
  misc_feature: Incyte ID No 5301066CD1
MFAEGEEMYLQGSSIWHPACRQAARTEDRNKETRTSSESIISVPASSTSGSPSRVIYAKLGGEILDYRDLAALPKSKAIYDIDRPDMISYSPYISHSAGDRQSYGEGDQDDRSYKQCRTSSPSSTGSVSLGRYTPTSRSPQHYSRPAGTVSVGTSSCLSLSQHPSPTSVFRHHYIPYFRGSESGRSTPSLSVLSDSKPPPSTYQQAPRHFHVPDTGVKDNIYRKPPIYRQHAARRSDGEDGSLDQDNRKQKSSWLMLNGDADTRTNSPDLDTQSLSHSSGTDRDPLQRMAGTAVTHDSPISKSDPLPGHGKNGLDQRNANLAPCGADPDASWGMREYKIYPYDSLIVTNRIRVKLPKDVDRTRLERHLSPEEFQEVFGMSIEEFDRLALWKRNDLKKKALLF

SEQ ID NO: 26 - DNA, 4519 bp, Homo sapiens
  misc_feature: Incyte ID No 2780338CB1
gagcttatggccaagttcacctgcaagttgcagagaggtttacatcgccaagctttggcctttcatttctgcaaagcccaggactctgatacttgtgtgttggagggtttattagaggtctagtagcccagatctgccgcagtggactactccaaggatatgaggacaagctaagggatccagcagtccaaagcctcttacagcctggggagtgcgagagaaacccagccgaagcatttaaaaggtgtgttctactccctcttctgggaatgaagcctccccagcaaagcctatacctgcttgttgattctgttgatgaagggtgtaacattactgaaggtgaacaaacgtctaccagcttatctgggactgttgcagcacttttagctggtcaccatgagttctttccaccatggctattgcttctctgttctgcccgaaagcagagtaaggctgttactaaaatgtttactggttttcgaaaaataagtttagatgaccttcggaaggcatatatcgtcaaggatgttcagcagtacattcttcatcgtttagatcaagaagaagctttgcgacaacacctcacaaaagaaactgcagagatgttaaatcaactgcacattaaaagcagtggatgctttctttacctagaacgagttttagatggagttgtagaaaattttattatgttaagagaaattcgtgacatcccaggaactctaaatggtttatatctctggctgtgccaaagactttttgtaagaaaacaatttgcaaaggttcagcctattttgaatgtgattcttgcagcctgccgacctttgaccataacggaattatatcacgcagtatggaccaaaaacatgtcgttaactttggaagattttcaacgcaagttagatatcctctccaaacttcttgttgatggactaggaaatacaaaaatactgtttcattatagttttgccgagtggcttctggatgtgaaacactgtactcagaagtatttatgtaatgcagcagaaggacacagaatgttggctatgagttatacctgtcaagccaagaatttaacaccattggaagcacaagaatttgcattgcacttaattaactcaaacttacaattagagacagcggagttagctctgtggatgatatggaatggtacacctgtcagagattccctttctactttgatacccaaggaacaagaagtgctacagctgttggttaaagctggggctcatgtcaacagtgaagacgatcgcacatcatgcatagttcgacaagccttagaaagagaggattccattcggacattattagataatggagcttcagtaaatcagtgtgattcaaatgggagaacattattggctaatgctgcatatagtggcagtcttgatgtagtcaatttacttgtctctaggggagcagatttagagatagaagatgctcatggacatacaccactcactctagcggctagacagggacataccaaggtggttaattgtttgattgggtgtggagcaaatattaatcatactgatcaagatggttggacagcattaagatctgctgcttggggtggccatactgaggtagtttctgcactactttatgctggcgtaaaagtggattgtgcagatgctgatagccgaaccgctttgagagcagcagcatggggaggacacgaggatattgtactgaatttgctacaacatggcgctgaagtgaacaaagctgataatgaaggtagaactgctttgatagcagcagcatacatgggacatagagagattgtggaacacctactggaccatggagcagaagtaaatcatgaggatgttgatggcaggactgcactctctgtagctgcactttgtgtgcctgcaagtaaagggcacgcatcagttgttagccttttaattgatcgaggtgctgaagtagatcattgtgataaagatggcatgactccactgctggtagctgcctatgaaggacatgttgatgtggttgacttgcttctagaagggggagcagatgtagatcacacagataacaatggccgtacacccctcttagcagcagcgtctatgggtcatgcatcagttgtaaatacacttttgttttggggtgcagctgtggatagtattgatagtgaaggtaggacagtcctcagtatagcttcagcacaaggaaatgttgaggtggtacgtactctactggatagagggttagatgaaaatcacagagatgatgctggatggacacctttgcacatggcagcttttgaagggcacagattgatatgtgaagcacttattgaacaaggtgctagaacaaatgagattgacaatgatggacgaatccctttcatattagcttcacaagagggtcattatgattgtgttcaaatattactggaaaacaaatccaacattgatcaaagaggttatgatggaagaaatgcactgcgggttgctgcattagaagggcacagggacattgttgaattgctttttagccatggtgctgatgttaactgcaaagatgctgatggtcggcctacactttatatcttggccttagaaaatcagcttacaatggccgaatattttttagaaaatggtgcaaacgtagaagcaagtgatgctgaaggaaggacagcacttcatgtgtcttgttggcaaggccatatggaaatggtgcaggtcctgatagcataccatgctgacgtcaatgctgcagacaatgaaaagcgctctgctttgcagtctgcagcctggcagggccatgtaaaagtggttcagcttctgattgagcatggtgctgtagttgaccatacatgtaaccaaggtgcaactgcactctgtattgcagcccaggaagggcacattgatgttgttcaggtcttattagagcatggtgctgatccaaaccatgctgatcaatttggacgcactgctatgcgtgttgcagccaaaaatggacattctcagataattaaattattagaaaaatatggtgcatctagtttgaatggctgttccccatctcctgttcacacaatggagcaaaaacctctacagtcattgtcttcaaaagtgcagtcattaacaattaaatcaaatagctctggtagtactggtggaggggatatgcagccttcgttacgtggtttacctaatgggcctactcatgcttttagttctccttcagaatctccagattctacagttgaccggcagaagtcatcactgtcaaataattccctgaaaagctcaaaaaattcatctttgagaactacttcatctacagcaacggctcaaacagtgccaattgatagctttcataacttgtcatttacagaacaaattcagcagcattcattgccacgcagtagaagtcgacagtcaattgtttccccatcttccacaacacagtccttaggacagagtcataattcaccaagtagtgaatttgagtggagtcaagtaaagcccagtttgaagtcaactaaagcaagtaaaggggggaaatcagaaaattctgccaagtctggatcagctgggaaaaaagcgaaacaaagtaattcttcacagccaaaggttttagaatatgaaatgactcagtttgatagaagaggacctatagccaaatccgggactgctgcaccacctaaacaaatgccagcagaatctcaatgcaaaattatgataccttcagctcagcaggaaattggtcgatctcaacagcagtttcttattcaccaacaaagtggggaacagaagaagagaaatggaataatgacaaatccaaattatcatcttcagagcaaccaggtttttcttggtagggtttcagtcccacgaacaatgcaagatagagggcatcaggaagtgttggagggatacccttcctcagagacagaattaagccttaaacaagctctgaagcttcagattgaaggttctgaccctagcttcaactataaaaaggaaacaccattataaaagtttcctattctgtgaaacagaagacattgtgatggagtggttcttcagctactggatggaaacatatgcctgttgatttgctgaaaaaacaaaaaaaatgaagaatgtgatctctgcagtacagttaccttaattactgtaatgtgcctaaatagtaaggctgccttctcaatgtaaccctctgtgcttaaaaaatttcattttgtgtgctttgtattcactacacaggaataagcactttttaaaaatgcagatacatactgcagttccctgataaaagctgaaaagaaaatttgagtattttaagttaagatgtgataaaaaatgtgcatgtgccataatcaaatatatatgaaaaggcagtgttccttgtatttatttttttttctttttgtggcaaaagaaacttaaacatactgtttcagtcacattgcattgtagtgtatggcctgtttcttgtatctttaaagacgtttctcaataaaacaaatctt

SEQ ID NO: 27 - DNA, 8008 bp, Homo sapiens
  misc_feature: Incyte ID No 2317440CB1
gcgcccggagccagcggcggcggcagcggctgaggcggctgcgggagcggagtggtcgcccagcgcgcggggggacgcgggctgcactcccgggcaggcgctctgcaatgagacagtaaatgcatcccccgatgtttgagaaacagtggcaggatctggaagttcatgttggtatcctttgtccatggtgctgcaggaggcttctcacatgctttgggatatcttcagggaaatacgctcttaatagaaactgagaatttaagacattctaagtgagactgtccacatcatctaggaaaatggtggccctgtccttaaagatttgtgtgcgccactgcaacgtggtgaagaccatgcagtttgaaccatctacagctgtgtacgatgcgtgtcgagtcattcgggaacgggtgcctgaggcacaaactgggcaagcttctgactatggactctttctttcggatgaagacccgaggaaagggatttggctggaagcgggcagaacactggattactacatgttgcggaatggggatattttggaatataaaaagaaacagagacctcagaaaatccggatgctggatggatctgtgaagacagtgatggtggatgattccaagactgtgggggagctcctggtcactatttgtagcagaataggaataacaaattatgaagaatactccttaatccaagaaactattgaagaaaagaaagaggaaggaacgggcacactcaaaaaagacaggacactgttacgagatgagaggaaaatggagaagttgaaggccaagctgcacacagatgatgacgtaaattggctggatcacagccgaacattcagagaacaaggagtagatgaaaacgaaacgttgctgcttagacggaagttcttttactctgatcagaatgtagattcgagagaccccgtgcagctgaacttgctttatgttcaggcacgggatgacatcctgaatggctctcaccctgtctccttcgagaaagcttgtgagtttggtggatttcaagcccagatacaatttggacctcatgtggaacataaacacaaacctggatttcttagtctgaaggaattcctgcccaaagaatatatcaagcagagaggagctgaaaagaggatctttcaggagcataagaactgcggagagatgagtgagatagaagccaaggtcaagtacgtcaaactcgcacggtccctccgcacaactgactcttggtatctcctgtttcaggagaagatgaaaggcaagaacaagctggtgcctcgcctgctggggatcaccaaagactcggtgatgcgcgtggatgagaagaccaaggaagtgctgcaggagtggcccctcaccaccgtcaagcgctgggcagcctcacccaagagcttcacactggattttggggagtatcaggaaagctactattcagtacaaaccaccgagggagagcagatatcccagctgattgcaggctacattgacatcatcctgaaaaagaaacaaagtaaagatcgatttggactagaaggtgatgaggagtcaaccatgttagaagagtccgtttccccaaaaaagtccaccatcttgcagcagcagttcaaccggaccgggaaggcagagcacggctcagtggcgctgccggccgtgatgcgctcgggctccagcgggcctgagaccttcaacgttggcagcatgccctcgccacagcagcaggtcatggttgggcagatgcaccgaggccacatgccgccactgacctcagcccagcaggccttgatggggaccatcaacacaagcatgcacgccgtccagcaggcccaggatgatctcagtgagctcgactcgctgccacctctcggccaggatatggcatctagggtatgggttcagaacaaagtcgacgaatccaaacacgaaatccattctcaagttgatgctatcacggccggaacggcttcagttgttaacctcacagctggtgaccctgcagacactgactacacagctgtgggatgtgcgatcaccactatttcttccaacctgacggagatgtccaagggtgtgaagctattggccgccctcatggatgatgaggtgggcagcggggaggacttgctcagagctgccaggaccctcgctggggcggtgtcagacttgctgaaagctgtgcagcctacttctggagagcctcgacagacagttttgactgctgctggcagcatcggacaagccagtggggatcttctgagacagattggagagaatgagactgatgagcgattccaggatgttttaatgagtttggccaaagctgttgccaatgcagctgccatgttggtactaaaggcaaagaatgttgcccaagtggccgaagacactgtcctacagaacagggtaattgctgctgccacccagtgtgccctctccacctcccagcttgtggcatgtgccaaggttgtgagccccactattagctcccctgtgtgccaggagcagctgattgaagcagggaagctggtggaccgctcggtggagaactgtgtccgtgcctgccaggcggccactaccgatagtgagctcctgaagcaggtcagcgcagcggccagcgtggtcagccaggccctccatgatctcctgcagcatgtgcggcagtttgccagccgaggcgagcccatcggccgctacgaccaggctactgacaccatcatgtgtgtcaccgagagcatcttcagctccatgggtgacgctggtgaaatggtgcgccaggcgcgggttctggcccaagccacatcagacctcgtcaatgccatgaggtcagatgcagaagccgaaatcgacatggagaattcaaagaagctcctggcagcagcaaaactcttagctgactccactgctcgcatggtggaagctgcaaagggggctgcagccaacccagagaatgaggaccagcagcaaaggctgagagaagctgcagaaggcctccgggtagcaaccaacgcagctgcccagaatgctattaagaaaaaaattgtcaaccgactggaggttgcagccaagcaggccgcagcggcagctacacagaccatcgccgcctcccagaatgcagctgtttccaacaagaaccctgcggcccagcagcagctggtccagagttgcaaggcagtggctgatcacatccctcagctggtccagggagtgagggggagccaagctcaagctgaagacctgagtgcccagctggctctcatcatctccagccagaacttcctccagcctggaagcaagatggtgtcctctgccaaagccgcagtgcccaccgtgagtgaccaggccgcagccatgcagctgagccagtgtgccaagaacctggccaccagcttggcggagctgcgtaccgcctcgcagaaggcccatgaagcttgtggtccgatggaaatcgattcagctctgaatacggtgcagacgcttaagaatgaactgcaggatgccaagatggcagccgtggagagccagctgaagccacttccaggggaaacgctggaaaaatgtgctcaggacctgggaagcacatccaaggcggtgggctcctccatggcacagctgctgacctgtgctgctcaaggcaacgaacactacacaggggtggctgctagagagacggcccaagctctgaaaacactggcccaggccgcccgtggagtggctgcatcgacaaccgaccccgcggccgcccatgccatgttagattctgctcgagacgtgatggagggctccgccatgctcattcaagaggccaagcaggccctgattgcacctggagatgcagagcgtcaacaaagactggctcaggtggctaaagccgtctcacactccttgaataactgcgtaaattgcctccctgggcagaaggatgtggacgtggccttgaagagcatcggggagtccagcaagaagctgcttgtggattcgctacctccaagcacgaagcctttccaggaagcccagagtgaactgaaccaggcagcagctgatctgaaccagtctgctggggaagtggtccatgccacccggggccagagtggagagttggctgcagcctctggaaagttcagtgatgattttgatgaattcctcgatgctggcattgagatggctggccaagctcagacaaaagaagaccagatccaagtgatagggaacctcaagaatatctcgatggcatccagcaagctgctgttagctgccaagtctctctctgtagatccaggagctcccaatgcgaaaaatctcctggctgcagctgcaagagctgtgacagagagcatcaatcaactcatcactctgtgtacccaacaagctccgggccagaaagagtgcgataatgccctgcgggagctcgagactgtgaaggggatgttggacaatcctaatgaacctgttagtgacctctcttactttgactgcattgagagtgtgatggaaaactccaaggttctgggtgaatcgatggcagggatttcacagaatgccaagaccggagacctccctgcctttggggaatgtgtggggattgcatccaaggctctctgtgggctgacagaggctgcagcccaggctgcatacttggttggcatctctgatccaaacagccaggcaggccaccagggcctggtggaccccatccagtttgccagggctaaccaggccatccagatggcatgccagaacttggtggaccctggcagcagcccatcacaggtcctgtcagccgccacaattgttgccaagcacacgtcagccttgtgtaatgcctgccgcatcgcctcatccaagacggccaacccagtagccaagaggcacttcgtccagtcagccaaggaagtcgccaacagcactgccaacctggtgaagaccatcaaggccctggatggggatttctctgaagacaaccgcaataagtgtcgcatcgccaccgcacccttgattgaagctgtggagaacctgacagcgttcgcctcaaaccctgagtttgtcagcattcctgcccagatcagctccgagggttcccaggcacaggaaccaatcctggtctcagccaagaccatgctggagagttcatcgtacctcattcgcactgcacgctctctggccatcaaccccaaagacccacccacctggtctgtactggctggacattcccatacagtgtccgactccatcaagagtctcatcacttctatcagggacaaggcccctggacagagggagtgtgattactccatcgatggcatcaaccggtgcatccgggacatcgagcaggcctcgctggccgccgtcagccagagcctggccacgagggacgacatctctgtggaggccctgcaggagcagctgacttcggtggtccaggaaatcggacaccttatcgatcccatcgccacagcggctcggggagaagcagctcagctgggacataaggtgacacaactggcaagctattttgagcccttgatcttagccgcagttggtgtggcctccaagattcttgatcatcagcagcagatgacggtgctggaccagaccaagactctcgcagagtctgccttgcagatgttgtatgcagccaaagaaggtggcggaaaccccaaggcacaacacacccatgacgccatcacagaggccgcccagttgatgaaggaagccgtggatgacatcatggtgacgctgaacgaagctgccagtgaagtggggctggttgggggcatggtggacgccattgcagaagccatgagcaagctggatgaaggcactcctccagaaccaaagggaacatttgtcgactatcagacgactgtggttaaatactccaaagccattgcggtgacagctcaggaaatgatgactaagtcggttactaacccggaggagttgggaggactggcttcacaaatgaccagtgactatgggcacctggctttccagggccagatggcagcagccacggcggaaccagaggagatcggattccagattcgcactcgtgtgcaggacctgggccacggctgtatcttcctggtgcagaaggcaggggccctccaggtctgccccacagacagctacaccaagagggagctgatcgaatgcgcccgtgccgtcacggaaaaggtctccttggtgctctcggctctccaggccgggaacaaaggaacccaggcatgcattacagccgccaccgctgtgtctgggatcattgccgacctggacaccaccattatgtttgcaacagcggggacgctgaatgcagagaacagtgagaccttcgcagaccacagggagaacattctcaagacggccaaggccttggtagaagacacgaaactacttgtgtcaggagctgcgtccactcctgacaagctggcccaggcggcccagtcctcagcagccaccatcacccagctcgcagaagtggtcaagctgggggcagccagcctgggctccgacgaccccgagacccaggtggttttgatcaatgccatcaaagatgtggccaaggccctttctgatctcatcagtgctaccaagggagctgccagcaagccagtggacgacccttccatgtaccagctcaagggggctgccaaggtgatggtgaccaatgtcacctcgctcctcaagactgtaaaggcagtggaggatgaggccacccggggcaccagggcgcttgaggccacaattgaatgcataaagcaggagcttacggtgttccagtcaaaagacgtacctgaaaagacatcatcacctgaagaatccataaggatgacgaaaggcatcaccatggcaacagccaaagccgtggcagctgggaactcatgtagacaggaggacgtgattgctactgccaacctgagccggaaagccgtgtcagatatgttgacggcttgcaagcaagcatccttccaccccgatgtcagtgacgaggtgagaaccagagccttgcgtttcgggacggagtgcacccttggctacttggacctcctggagcacgtcttggtgattcttcagaaaccaaccccagaattcaagcagcagctggccgctttctccaagcgagtcgccggcgctgtgacagagctcatccaggcggcggaagccatgaaaggaacagagtgggtggatccagaagacccaactgtcattgcagaaacagagttactgggggctgcagcatccatcgaagctgctgctaagaagttagagcaactgaagccaagagcaaaaccaaaacaagcggatgagaccctggactttgaggaacagatcttggaagctgctaaatccattgctgctgccacaagcgccctggtcaaatcggcctcagcagcccagagggagctggtggcccaaggaaaggtgggctccatccctgccaatgctgcagacgacggacagtggtcacaggggctgatttctgctgcccggatggtggcggctgcgaccagcagtctctgtgaggcggccaatgcctccgttcagggacacgccagcgaggagaagctcatctcatctgccaagcaggtcgccgcttccacggctcagctgctggtggcctgcaaggtgaaggccgaccaggattcagaggccatgaggcggctacaggcggcaggaaatgctgtgaaaagagcctcagacaatcttgtccgtgcagcccagaaggcagcttttggcaaagctgatgacgacgatgttgtagtgaaaaccaagtttgtggggggcattgctcagatcatcgccgcccaggaagaaatgctaaagaaagagcgagaactggaagaagcaaggaaaaaactggcccaaatccgccagcagcagtataagtttttacccaccgagctgagggaagatgagggctaaaggtgcgagcccagatggcgagccccaggggatggccctggctgaactggacagacagtgttcctgagaggctgggcact

SEQ ID NO: 28 - DNA, 2160 bp, Homo sapiens
  misc_feature: Incyte ID No 3013470CB1
ctcagcaaaccactgaggttcctggaaaaaaaaaaaaaatatctcaaggtaaaataatatttagaaaaaataatgtcagagcacagcagaaattcagatcaagaagaacttctcgatgaggagattaatgaagatgaaatcttggccaacttgtctgctgaagaactgaaagaactgcagtcggaaatggaagtcatggcccctgaccccagccttcccgtgggaatgattcagaaagatcaaactgacaagccaccgacaggaaacttcaatcataaatctcttgttgattatatgtattgggaaaaggcatccaggcgcatgctggaagaggaacgagttcctgtcacctttgtgaaatccgaggaaaagactcaagaagagcatgaagaaatagaaaaacgtaataaaaatatggcccagtatttaaaagaaaagctcaataatgaaatagttgcaaataaaagagaatcaaagggcagcagcaatatccaagaaacagatgaagaagatgaagaagaagaagatgatgatgatgacgacgaaggagaagatgatggtgaagagagtgaagaaacgaacagagaagaggaaggcaaagcaaaggaacaaattagaaattgtgagaacaactgccagcaggtaactgacaaagcattcaaagaacagagagacagaccagaggcccaagaacaaagtgagaaaaaaatatcgaaattagatcctaagaagttagctctagacaccagctttttgaaggtaagtacaaggccttcaggaaaccagacagacctggatgggagcttgaggagagttaggaaaaatgatcctgacatgaaggaactcaacctgaacaacattgaaaacatccccaaagaaatgttactggactttgtcaatgcaatgaagaaaaacaagcacatcaaaacattcagtttagccaatgtgggtgcagatgagaatgtagcatttgccttggctaacatgttgcgtgaaaatagaagcatcaccactctcaacatcgagtccaatttcatcacaggtaaagggattgtggccatcatgaggtgtctccagtttaatgagacgctaactgagcttcggtttcacaatcagaggcacatgttgggtcaccatgctgaaatggaaatagccaggcttttgaaggcaaacaacactctcctgaagatgggctaccattttgagcttccgggtcccagaatggtggtcactaatctgctcaccaggaatcaggataaacaaaggcagaaacgacaggaagagcaaaaacagcagcaactcaaggaacagaagaagctgatagccatgttagagaatgggttggggctgccccctgggatgtgggagctgttgggaggacccaagccagattccagaatgcaggaattcttccagccaccgccacctcggcctcccaacccccaaaatgtcccctttagtcaacgcagtgaaatgatgaaaaagccatcgcaggccccgaagtacaggacagaccctgactccttccgggtggtgaagctgaagagaatccagcgcaaatctcggatgccggaagccagagaaccacccgagaaaaccaacctcaaagatgtcatcaaaacgctcaagccagtgccgagaaacaggccacccccattggtggaaatcactcccagagatcagctgctaaacgacattcgtcacagcagtgtcgcctatcttaaacctgtgcaactgccaaaagaactggcgtaagaggcaacagagccatctagaagaacaagaaatggaaatagtgactcttggattacagcatggagactatgtcagcagcaatactttaggatccacgtggcagaactggaaacaatgctaccatctgataagggtatttgtaaaaggcagaatgtttgggccatgaagaagtaggggctgaagaggaaggtggaaggagataaaatataatatttagaggcaatattttctacttgcaatcaatttgagtgactcaggtgaaatttagagtcatatttcccgaagcagaagttaaagaaaattttttaaacatttgctttattattgttttcttctggtaaataataaatataacagaagtgttaactattgtattcccatttttttctcctccatcttcctgct

SEQ ID NO: 29 - DNA, 2992 bp, Homo sapiens
  misc_feature: Incyte ID No 1738823CB1
gagctcggctcactagttacggcgcagtgtgctggacagagcggtgcggcgcgattgttcctgcgcttcggggctgcccgccgcgtccccgcgcgccgccgacccgcgcccgctggcttccgcgcctctgccggggagcggcccgcggatgcgcaccccgccagctctcgggagccagggcagcgaggtcacaggccccacctttgctgatggcgagctcatccccagggaacccggcttttttcccgaggacgaggaggaggctatgacgctggcgccacctgagggcccccaggagttgtacacagacagccccatggagagcactcagagcctggaggggtctgtcgggagtcctgccgagaaggacgggggacttgggggcctgtttctgccagaagacaatgcggggcagacgcctaggaaaatgcggcacgtgtacaacagcgaattgctagatgtttaccgctctcaatgctgcaagaaaatcaacctgctcaatgacttggaagcccgactgaaaaacctgaaggccaacagccccaaccgaaagatctccagcacggcctttggacggcagctcatgcacagcagcaacttcagcagcagcaatggcagcaccgaagacctgttccgggacagcattgactcttgcgacaatgacatcacagagaaggtaagcttcctggaaaagaaggtgacagagctggagaatgacagcctgaccaatggggacctgaagagcaagctgaagcaagagaacacacagctggtgcacagggtgcatgagctggaggagatggtgaaggatcaggagaccacggccgagcaggctctggaggaggaggcgcggcgccaccgcgaggcctacggcaagctggagagggagaaggctaccgaggtggagctgctcaatgccagggtgcagcagttggaggaagaaaatacagagcttagaacaacagtgactcggctcaagtctcaaacagagaaactggatgaggagcggcagcgcatgtctgaccgtctggaggacaccagcctgcggctcaaagatgagatggacctgtacaagcgcatgatggacaagctgcgacagaaccgccttgagttccagaaggagcgggaggcgacgcaggagctcatcgaggacttgcggaaggagctggagcacctgcagatgtacaagctggactgcgagcggccaggcaggggccgcagtgcctcctctggcctaggcgagttcaatgccagggcccgcgaggtggagctcgagcacgaggtcaagcggctcaagcaggagaattataagctgcgggatcagaacgacgacttgaatgggcagattttgagcctcagcctctacgaagcaaaaaacctctttgctgcccagactaaagcccagtctctggctgcggagatagacaccgcctcgcgcgatgagctaatggaagccctgaaggagcaggaggagatcaacttccggctgaggcagtacatggacaagattatcctcgccatcctggaccacaatccctccatcctcgagatcaaacactaaggcacggggctggctgcagagcagccttaggaccctgggaccaagggcagaccctgcccaaggatgcaggcctaagccgggcctcacactcacactgtaaatgtctctctggccaccatgcgttacgtgtacccgtgtatatgtggggaggctgtgcacacgagcgaggggtgagtggccgtggctgtgggcagcatccacacggttagccgtgcatgcactttgtggcccctttgcaaggggcagagggtactggaagtgggaggaggcaaggtctgctatcaggagttactgtaaaaacaagaactggaagctcgtgtttccggtactgggtaaaatgattctacctctggggataaggattcacattcgctctagtacgatgggctctttcaccccactcctagtccccttgggagtgggagcaaaattgtgatctttcctaggagtttgaatgccccatatttgtgtcctcgccagctctttggccacctttcaagccccagtgttcaagctcagagaggatgaaggggcatctggagggtccatgagatggggccctcaccaaatgccttggccaccggccagcagccctctgtgatgtgtgtgcagagtgaacagaggactcaggtttcatagcagctagtgcggggcatcctcatcctgtacctatagagttcgtgcactcccccatccatgctacctacctccccgtttctgtttcagttttaagacaatgaagcagcattcactgcgtcattgtaacatgaagggataaaaatgaaggagaaggagggtttgggatgggtgtctaaggcaggagttgacatcgggcaaccaaagaaatgagttttggggaggaacacaactcccatcccagtcagtttccttcccatggctgcatcttaagatggatgcacggagaaaccgtctgcctggctccctgtcttcattctccacgcagcctggtgatggcaggcctgggtttggatttcagaatcctagctccgggctccactcgtgtggcagcaagactgcttcgttccagcgtttagaaacacacctgtatttgattctcagccaggggagcactcgctgcactggtgggaggcggttgggaaagttgcaggaaaaccttagtcttccatccttctgacccatggtggaaattcacaccatggatttttaatggatctttgttctaggcagctgggaatagacatggtacttaccttagagttttccaatttatctcaattttatatggcttgtgattcattttcttaatccaaatatatataaacgtgtgtggtcataaaaaaaaaaaaaaaaaaaaaaaaaaaaaaaaaaaaaaaaaaaatagtaagaaaataggatgtaaaaactagacatacaaagatgatgtagaaaaataaaggcggggctcgcccc

SEQ ID NO: 30 - DNA, 3225 bp, Homo sapiens
  misc_feature: Incyte ID No 4184551CB1
ctccgcaggggtttggggaaacggccgctgagtgaggcgtcggctgtgtttctcaccgcggtcttttcctcccactcttggctggttggaccccgctatggaaaagttggcccctgagccagagctccagcagccttgttagggcgtggcctgaggcttggataagtgggatgtaaaacgaagatcaggagcagatttgaagaattacaaagtgaattggtgccagtcagcatgtcagagacagaccacatagcctctacttcctctgataaaaatgttgggaaaacacctgaattaaaggaagactcatgcaacttgttttctggcaatgaaagcagcaaattagaaaatgagtccaaactattgtcattaaacactgataaaactttatgtcaacctaatgagcataataatcgaattgaagcccaggaaaattatattccagatcatggtggaggtgaggattcttgtgccaaaacagacacaggctcagaaaattctgaacaaatagctaattttcctagtggaaattttgctaaacatatttcaaaaacaaatgaaacagaacagaaagtaacacaaatattggtggaattaaggtcatctacatttccagaatcagctaatgaaaagacttattcagaaagcccctatgatacagactgcaccaagaaatttatttcaaaaataaagagcgtttcagcatcagaggatttgttggaagaaatagaatctgagctcttatctacggagtttgcagaacatcgagtaccaaatggaatgaataagggagaacatgcattagttctgtttgaaaagtgtgtgcaagataaatatttgcagcaggaacatatcataaaaaagttaattaaagaaaataagaagcatcaggagctcttcgtagacatttgttcagaaaaagacaatttaagagaagaactaaagaaaagaacagaaactgagaagcagcatatgaacacaattaaacagttagaatcaagaatagaagaacttaataaagaagttaaagcttccagagatcaactaatagctcaagacgttacagctaaaaatgcagttcagcagttacacaaagagatggcccaacggatggaacaggccaacaagaaatgtgaagaggcacgccaagaaaaagaagcaatggtaatgaaatatgtaagaggtgagaaggaatctttagatcttcgaaaggaaaaagagacacttgagaaaaaacttagagatgcaaataaggaacttgagaaaaacactaacaaaattaagcagctttctcaggagaaaggacggttgcaccagctgtatgaaactaaggaaggcgaaacgactagactcatcagagaaatagacaaattaaaggaagacattaactctcacgtcatcaaagtaaagtgggcacaaaacaaattaaaagctgaaatggattcacacaaggaaaccaaagataaactcaaagaaacaacaacaaaattaacacaagcaaaggaagaagcagatcagatacgaaaaaactgtcaggatatgataaaaacatatcaggagtcagaagaaattaaatcaaatgagcttgatgcaaagcttagagtcacaaaaggagaacttgaaaaacaaatgcaagaaaaatctgaccagctagagatgcatcatgccaaaataaaggaactagaagatctgaagagaacatttaaggagggtatggatgagttaagaacactgagaacaaaggtgaaatgtctagaagatgaacgattaagaacagaagatgaattatcaaaatataaggaaattattaatcgccaaaaagctgaaattcagaatttattggacaaggtgaaaactgcagatcagctacaggagcagcttcaaagaggtaagcaagaaattgaaaatttgaaagaagaagtggaaagtcttaattctttgattaatgacctacaaaaagacatcgaaggcagtaggaaaagagaatctgagctgctgctgtttacagaaaggctcactagtaagaatgcacagcttcagtctgaatccaattctttgcagtcacaatttgataaagtttcctgtagtgaaagtcagttacaaagccagtgtgaacaaatgaaacagacaaatattaatttggaaagtaggttgttgaaagaggaagaactgcgaaaagaggaagtccaaactctgcaagctgaactcgcttgtagacaaacagaagttaaagcattgagtacccaggtagaagaattaaaagatgagttagtaactcagagacgtaaacatgcctctagtatcaaggatctcaccaaacaacttcagcaagcacgaagaaaattagatcaggttgagagtggaagctatgacaaagaagtcagcagcatgggaagtcgttctagttcatcagggtccctgaatgctcgaagcagtgcagaagatcgatctccagaaaatactgggtcctcagtagctgtggataactttccacaagtagataaggccatgttgattgagagaatagttaggctgcaaaaagcacatgcccggaaaaatgaaaagatagaatttatggaggaccacatcaaacaactggtggaagaaattaggaaaaaaacaaaaataattcaaagttatattttacgagaagaatcaggcacactttcttcagaggcatctgattttaacaaagttcatttaagtagacggggtggcatcatggcatctttatatacatcccatccagctgacaatggattaacattggagctctctttggaaatcaaccgaaaattacaggctgttttggaggatacgttactaaaaaatattactttgaaggaaaatctacaaacacttggaacagaaatagaacgtcttattaaacaccagcatgaactagaacagaggacaaagaaaacctaaaacaagcctcttgctcagtaaagagacaaaagccacacaggagtaggtgccactgacctctattgttggagactttgttccactttttgtttcagccagtaaaaatattgttttgcttcatctgtacacaaaaaaatacccttttacaatatgaatgcattgctgtatatactgtaagactgaaagctttgatgaaatttgtttttgtatggtgcaatatgacagcctgtcattgaatctaaacaacttaatttgcttgtattcataagaagtgttgaacattacaagggcttttat

SEQ ID NO: 31 - DNA, 2787 bp, Homo sapiens
  misc_feature: Incyte ID No 70042484CB1
tccaacaggctccccaccatgatgaagacggagccacgggggcccgggggtcccctccggagcgcctccccgcaccgcagcgcctacgaggcgggcatccaggcgctgaagccgcccgacgcgcccgggcccgacgaggcacccaagggggcccaccacaagaaatatggctccaacgtccaccgcatcaaaagtatgttcctgcagatgggcacgacggcggggccctcgggcgaggcgggcggcggcgcgggcctggccgaggccccacgggcgtccgagcgcggcgtgcgcctgtcgctgccgcgggccagcagcctgaacgagaacgtggaccacagcgccctgctgaagctgggcaccagcgtgtcggagcgcgtgagccgcttcgactccaagcccgcgccctccgcgcagcctgcgccgccgccgcacccgccgtcccggctgcaggagacgcggaagctgttcgaacggagcgccccagcggccgcaggcggcgacaaggaggccgcggcgcggcggctgctgaggcaggagcgcgccggcctgcaggaccggaagctggacgtcgtggtgcgcttcaacggcagcaccgaggcgctggacaagctggacgctgacgccgtgtcccccacggtcagccagctcagcgccgtcttcgagaaggccgactcgaggaccggcctccaccgcgggcccgggctccccagggccgcaggggttccccaggtcaactcgaagctggtcagcaagcggtcccgggtgttccagcccccgccgccgccgccgcccgccccgtcgggggatgccccggccgagaaagagcgatgccccgcagggcagcagcccccgcagcaccgagtggcccctgcccggccgccccccaagccccgggaggtgcgcaagattaagccggtggaggtggaggagagcggggagtcggaggccgagtcggcgcccggggaggtgatccaggccgaggttacggtccacgcggccctggagaatggcagcaccgtggcaactgcagccagccccgcgcccgaggagccaaaggcccaagcggccccggagaaggaggcggcggcggtagcgccgccagagaggggggtgggcaatggccgggccccggacgtggcccctgaggaggtagatgaatccaagaaggaggacttctcggaggcggacttggtggacgtgagcgcctacagtgggctcggggaggactctgcgggcagtgccctggaggaggacgacgaagacgacgaggaggatggggagcccccctacgagcccgagtcggggtgcgtggagatcccggggctgtcggaggaggaggacccagccccgagccggaagatccatttcagcacggcgcccatccaagtgttcagcacttactccaacgaggattacgatcgtcgcaacgaggatgtggatcccatggcagcctctgctgagtacgagctggagaagcgtgtggagaggttggagctgttccctgtggagctggagaaggactccgagggcctgggcatcagcatcatcggcatgggcgccggggcagacatgggcctggagaagctgggtatcttcgtcaagaccgtgacggagggtggtgcggcccatcgggatggcaggatccaggtgaatgatctcctggtggaggtggatggaacaagtctggtgggagtgacccagagcttcgcggcgtctgtgctccggaacaccaagggccgagtgcggtttatgattggccgggagcggccgggagagcagagcgaagtggcccagctaattcagcagactttggaacaggagcgatggcagcgggagatgatggagcagagatacgcccagtatggggaggatgacgaggagacgggagagtatgccactgacgaggatgaggagctgagccccacgttcccgggtggtgagatggccatcgaggtgtttgagctagcggagaacgaggatgcactgtcccctgtggacatggagcccgagaagctggtgcacaagttcaaggagctccagatcaagcatgcggtcactgaggcagagatccagcagctgaaaagaaagctgcagagcctggagcaggagaaggggcgctggcgggtggagaaggcgcagttggagcagagtgtggaggagaacaaggagcgcatggagaaactggaaggctactggggtgaggcccagagcctgtgccaggctgtggacgagcacctgcgggagactcaggcgcagtaccaggccctggagcgcaagtacagcaaggccaagcgcctcatcaaggactaccagcagaaggagatcgagttcctgaaaaaggagactgcacagcgtcgggttctggaggagtcggagctggccagaaaggaggagatggacaagctcctggacaagatctcagaactggaaggaaacttgcaaacactgaggaattccaattctacttaacaggaatcattccatgactggacaataattaacccccctcccattgtcctccctcccctgtcctcaacaccccacccctcccccttccagcctggggacaggtgccccgactccccccacccctccaccccacctcccccagcttcagggaccagagggctcatatcacaggcccccttaagatggcctgggcagacagaggtggctagaagggcagcctctttcttgccccatggggctgaggcacagagccgagggctgccgagggctggcctggtggtcgacggacacaagcacctgcagatcaaactgcca

SEQ ID NO: 32 - DNA, 2444 bp, Homo sapiens
  misc_feature: Incyte ID No 3236274CB1
ggcgtggacgcgcgcggggccgccgcgggcacggagtggccgccgcgtcgcctgagcccagagcccgggagtgctctcggccgccgcgtctcctgccctctgtccttccaacccagccctcggctgagccgcgccgcaccatgcccgccgtggacaagctcctgctagaggaggcgttgcaggacagcccccagactcgctctttactgagcgtgtttgaagaagatgctggcaccctcacagactataccaaccagctgctccaggcaatgcagcgcgtctatggagcccagaatgagatgtgcctggccacacaacagctttctaagcaactgctggcatatgaaaaacagaactttgctcttggcaaaggtgatgaagaagtaatttcaacactccactatttttccaaagtggtggatgagcttaatcttctccatacagagctggctaaacagttggcagacacaatggttctacctatcatacaattccgagaaaaggatctcacagaagtaagcactttaaaggatctatttggactcgctagcaatgagcatgacctctcaatggcaaaatacagcaggctgcctaagaaaaaggagaatgagaaggtgaagaccgaagtcggaaaagaggtggccgcggcccggcggaagcagcacctctcctcccttcagtactactgtgccctcaacgcgctgcagtacagaaagcaaatggccatgatggagcccatgataggctttgcccatggacagattaacttttttaagaagggagcagagatgttttccaaacgtatggacagctttttatcctccgttgcagacatggttcaaagcattcaggtagaactggaagccgaggcggaaaagatgcgggtgtcccagcaagaattactttctgttgatgaatctgtttacactccagactctgatgtggccgcaccacagatcaacaggaacctcatccagaaggctggttaccttaatcttagaaacaaaacagggctggtcaccaccacctgggagaggctttatttcttcacccaaggcgggaatctcatgtgtcagcccaggggagccgtggctggaggtttgatccaggacctggacaactgctcagtgatggccgtggattgcgaagaccggcgctactgcttccagatcaccacgcccaatggaaaatcgggaataatcctccaggctgagagcagaaaggaaaatgaagagtggatatgtgcaataaacaacatctccagacagatctacctgaccgacaaccctgaggcagtcgcgatcaagttgaatcagaccgctctgcaagcagtgactcccattacaagttttggaaaaaaacaagaaagctcatgccccagccagaacctgaaaaattcagagatggaaaatgaaaatgacaagattgttcccaaagcaacagccagtctacctgaagcagaggagctgatcgcgcctggaacgccgattcaattcgatattgtgcttcctgctacagaattccttgatcagaacagagggagcaggcgtaccaacccttttggtgaaactgaggatgaatcatttccagaagcagaagattctcttttgcagcagatgtttatagttcggtttttgggatcaatggcagttaaaacagacagcactactgaagtgatttatgaagcgatgagacaagtattggctgctcgggctattcataacatcttccgcatgacagaatcccatctgatggtcaccagtcaatctttgaggttgatagatccacagactcaagtatcaagggccaattttgaacttaccagtgtcacacaatttgctgctcatcaagaaaacaagagactggttggttttgtcatccgtgttcctgaatccactggagaagaatctctgagtacatacatttttgaaagcaactcagaaggcgaaaagatatgttatgctattaatttgggaaaagaaattattgaggttcagaaggatccagaagcactggctcaattaatgctgtccataccactaaccaatgatggaaaatatgtactgttaaacgatcaaccagatgacgatgatggaaatccaaatgaacatagaggcgcagaatccgaagcataactcacttgcgcctgtgggggaagagcaaacaggaaggagagctacctcctaagggttttaacgtctctgacatacaggcacactgacctgatttccgaaggctgacaatcgtttgtggaatgtaatcttgatgccttgatactgagacttgggagggaaactaagaaatggttgacagcgttcccacccatctacaatgttattttaggtgctttgtggtaagtcttttttcttagattgcgctaaaatttcttagattgttcagcgctcagaaccaagtttgaaaaatgcattgttcatatgaatgtc

SEQ ID NO: 33 - DNA, 2605 bp, Homo sapiens
  misc_feature: Incyte ID No 7179725CB1
aaccacattggccggtcaggatcggaggctgaaggaagattatagagacttgctttagaaccacaagaagaaagaggaggccggcttttcagctagcatcatggcgtggccgtgcatcacgagggcctgctgcatcgcccgcttctggaaccagttggacaaagcggacatcgctgtgccgctggttttcaccaagtactcggaggccaccgagcacccgggcgccccgccgcagccaccgccgccgcagcagcaggcgcagccggcgctcgcgcccccctcggcgcgcgcggttgccatagagacgcagccagcccagggcgagttggatgcagttgcccgggcaacggggccagcgcctgggcctaccggcgagcgcgagccggcggcgggccccggccggagcgggccgggcccgggcctgggctccggctccacctccggccccgcggactcggtgatgcggcaggattaccgagcctggaaggtgcagcggcccgagcccagctgccggccgcgcagcgaataccagccctccgacgctcccttcgagcgcgagacccagtaccagaaggacttccgcgcctggccgctgccgcgccgcggggaccacccgtggatccccaagcccgtgcagatctctgcggcctcccaggcgtcggcgcccattctcggggcgcccaagcgccggccgcagagccaggagcgctggccagtgcaggccgccgctgaggcccgggagcaggaggcggcccccggcggagcgggtggcctggcggccggaaaggcgtccggggcggacgagcgcgacacgcgcaggaaggccgggcctgcctggatggtgcgccgcgccgagggcctggggcacgagcagacgccgctgcccgcggcccaggcccaggtccaggccaccggccccgaggctggcagggggcgcgccgcggcggacgccctcaaccggcaaatccgcgaggaggtggcgagtgcagtgagcagctcctacaggaatgaattcagggcatggacggacatcaagcctgtgaaaccaataaaggccaagccccagtacaagcccccagatgataagatggttcatgagaccagctacagtgctcagttcaaaggagaggccagcaagccaacaacagctgacaataaggtcattgatcgcagaagaatacgcagcctctacagcgaacccttcaaggaacccccaaaggtggaaaaacctagtgttcagagttccaaaccaaaaaagacctcagcgagccataagcccacgaggaaggccaaagacaagcaggcggtgtcaggccaggctgccaagaaaaagagcgcggagggcccgagtaccaccaagccagacgacaaggagcaaagcaaagagatgaacaataaactggctgaggcgaaagagagcctggctcaacccgtcagtgattcaagtaagactcaaggtcctgtagccacagagccagacaaggatcaaggttctgtggtcccaggccttctgaaaggtcaaggtcctatggtgcaagagcctctgaagaagcaaggttctgtggtcccagggcctccaaaggatctaggtcccatgatcccattaccagtcaaggatcaagatcacacggtccctgagcctttaaagaatgaaagccctgttatctcagcaccagtcaaggaccaaggtccctcggtcccagttcctccaaagaatcaaagtcctatggttccagcaaaagttaaggatcaaggctctgtggtaccagagtctctaaaggatcaaggtcctaggattcctgagcctgtgaagaatcaagctcctatggtcccagcacctgtcaaggatgaaggtcccatagtcccagcacctgtcaaggatgaaggtcccatggtctcagcacctatcaaggatcaagatcccatggtcccagagcatccgaaggatgaaagtgccatggccacagcacccataaagaatcaaggttccatggtctctgagcctgtaaagaatcaaggtttagtggtctcagggccagtcaaggatcaagatgttgtagtcccagagcatgcaaaggttcacgattctgcagttgtggcacctgtaaagaatcaaggtcctgtggtccccgagtccgtgaagaatcaagaccccattctcccagtactagttaaggatcaaggccccacagtcctacagcctccaaagaatcaaggtcgtatagtccctgaacctctgaagaatcaagttcctatagtcccagtgcctctgaaggatcaagatcctctggtgccagtaccagcaaaggaccaaggtcctgcagtccctgaacctctgaagactcaaggtcccagggaccctcagctacctactgtctcacctctaccccgagtcatgatcccaactgccccccatacggaatacattgagagctccccttgacactcaccccttgacacaccaatgaaggagctgacagtgagagtgctcccctcccaggggcagtgaagacacatatttaatctgcatgaaacatgtacagtagtcttgctggaatctaataaaaatggtccctctggctcagcaaaaaaaaaaaaaaggggg

SEQ ID NO: 34 - DNA, 3716 bp, Homo sapiens
  misc_feature: Incyte ID No 1966217CB1
gtgtaatcttcagcctccctgaagctttacagccaggagtgagattttatcacatgaactttaaaagagtaaagccaaatatctttttattaaaccagagtagattaattatcagtatatgtgaaatcatgtccactgatcaaggcagcagtacctgtccaaagatcgctttagttccaccttgctccacaagcagcacaaccacactggttggtgagaatgtatctgaagaagaggctcaggaataaatgggaacaggccagcaaaacactcagctgcaagtccaaagccacaagtgcctccaaagccattacacctgcagaattcaccttcgtccaatatacaccaaacccccaggcataaagctttacctagtgcaaaaccaaggatggaggaaattaaacctgcctctgcttcttgtgtctcaaaagaaaaacccagtaaggtatcagatctcatcagtcgctttgaaggaggcagctcattatcaaattatagtgatttgaagaaagagtctgctgtgaacctaaatgctcctagaaccccaggaaggcatggattgacaaccacacctcaacaaaaactcctctcccagcacttgccacagaggcagggaaatgatacagataagactcagggtgcacagacttgtgtggccaacggtgtaatggcagcacaaaaccagatggaatgtgaggaggagaaagctgccactcttagctcagatacttctattcaagcttctgaacccttgcttgatacgcacatagtgaatggagaaagagatgaaactgccacagctcctgcatcacccacaacagacagctgtgatggaaatgcttctgacagtagctacaggactccaggcataggcccagtgctccccctagaagaaagaggggcagaaacagaaaccaaggtacaagagagggaaaatggggaaagccctctggaactggagcagctggaccagcaccatgagatgaaggagactaatgagcaaaaacttcacaaaatagccaatgaacttttgcttactgaaagagcttatgtcaaccgacttgacctcttagatcaggtattttattgcaaactgttggaagaagcaaaccgaggctcgtttccagcagagatggtgaataaaatcttttctaatatttcatcaataaatgccttccatagtaaattcctcttgccagagctggagaaacgaatgcaagaatgggaaactactcctagaattggagacatccttcagaaattggcaccattccttaagatgtatggagaatatgtgaaaggatttgataatgcaatggaattggttaaaaacatgacagaacgtattccccagttcaaatcagtggttgaagaaattcagaaacagaaaatctgtgggagcttaactttgcagcatcacatgctagaacctgttcagcggattccccggtatgagatgctccttaaggactatctaaggaaattgcctcctgattccctggactggaatgatgctaaaaaatcacttgaaattatatctacagcagcaagccattctaatagtgcaataaggaaaatggagaacctaaagaaactcttagagatttatgaaatgttgggagaagaagaagacattgtaaacccttcaaatgaactaataaaagaaggacagatcctcaaactagctgctcggaacacttcagcacaagaacgctaccttttcttattcaacaacatgttgctgtactgtgtgcccaaattcagcttggtaggctctaaattcacagttcgaaccagggttggcattgatggaatgaaaattgtagagactcaaaatgaagaatatccacatactttccaggtgtctgggaaagagagaacactggaactgcaggccagttctgcgcaagacaaagaagaatggatcaaggcccttcaagaaaccatcgatgcttttcatcaaaggcatgaaaccttcagaaatgcaattgcaaaggataatgacattcactcagaggtttctactgctgagctagggaaaagagccccaagatggatccgagataatgaagtgacaatgtgtatgaaatgtaaagaacctttcaatgcactgacacgaaggaggcatcattgtcgagcatgtggatatgtggtttgttggaaatgctccgactacaaagctcaacttgaatatgatggtggtaaattgagcaaagtttgtaaagactgttatcaaatcataagtggattcacagacagtgaagaaaagaaaagaaaaggaattttagagattgaatcagcagaagtatctggaaacagtgtggtgtgcagctttcttcagtatatggagaagtcaaaaccttggcagaaagcttggtgtgtgatccccaagcaagaccctcttgtgctgtacatgtatggtgccccccaggacgtcagagcccaggccaccattccacttctgggctatgtggtggatgaaatgccaaggagcgcagacctgccacacagtttcaaactgacccagtctaagtccgtgcacagctttgctgcagacagtgaggaactgaagcagaagtggctgaaagtcatccttttagctgtcacaggtgagacaccaggtggtccaaatgagcatccagccaccttggatgatcatcctgaacctaagaaaaaatcagaatgctgaactcctccaggaccagccatggtgtggaggtctcaggacttacagctcaagacattcccagctcttcttacacatctgctagcactttatgttgaaaaatataggcccataaatgcatcttttgaggactattttcctatgtttatgtactcttagtgaaattagtgtgcagagtcattctaccgataaagttttgaaataatgtgaaaactggagcattttttgagctattccttgaatatgtgcttttttgtcttgaagaaatggtgtatcaattgattctgtcaccgtcaggttagaatgagcacttccatttaagaaatcctttcatgtcttcttctctttcacatgtaggacctggaacagtttgaaagatatacctccatgttgccaaaatagatccatggtgaaaaatacagggacagttgaggctattgtattaacttatttaatttagtttataaatctctagctgcataaatgatgtctgttcttttaaaacaaaagaaaaaggacaaattgttggtgttcagatttccgatttataaagaaaagataacttgtttttgtagaaatactcctaagaatgtcttaagtatatagcaattatgtatatatagtattaaatatatatattatacatgcttttaggttcacattcatctctaatttattttttaaatataaagcatggtttatcgtggaattgagcaatgttctaaactgaagaaatgttttggtgatttatgttttgctgattggcatttgagggtattgatatttttataataagcggtaatttatgtggcatgggatatatttgtgaattccaacagtacttttaaagtaccattttttgtttctgtgcctatttaaaacagtgcctctcttagaaggtgctattaatataagatgagggttcagttaatgcttcagtcttgattattctaagattaaagtgcattttggtcactgggaaaaaaaaaaaaaaaaaaaaaaaaaagaaaaacaaaaaaaagaggcgcgg

SEQ ID NO: 35 - DNA, 1372 bp, Homo sapiens
  misc_feature: Incyte ID No 1598186CB1
cactgggcttttctatagtgtcacctaaatgcggccgcatttaggtgacactatagaagagcccagtgtgctggaaagggtactttgccctcggagcgaaggaggctccagaactggtagagccgggccatcgggctgggcacctccccgcggcgcccgcagcgcggagtccactgaccggctcaaaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcggccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggaggacgcttgtgctatggagggaatgagattgtcactggaagctttggaggggatggtggagggcgccaagcggagggacaggaggaagaccaggagacccatccagccaagctggtgagacaacctctgatcctgaggaccggccgcccaccaagggttgggcccccggggccaggttgatctaccgacaccttccact

SEQ ID NO: 36 - DNA, 1562 bp, Homo sapiens
  misc_feature: Incyte ID No 7493044CB1
ccagtgacaatacatcttttggataaggggattttggcactcagaaagaagctgcttgggccataagtaacttaaaagttagtggaaggaaagatcaagtggcttaccttatccagcagaatgttatcccacctttttgcaacttgctgactgtaaaagatgcacaagtggtcgtccgcaaagcctgagtcctgtcctctcactctcctccccagacagcatgagcttcaacacttgctccaccttctccacctgctccaccttctccaccaactaccagtccctggggcgcctggacagtcagcgcgtggccagcatctatgcaggtgctgggggctctggttcccagatctccctgtcccactccaccagcttacagggtggcatggggtccagaggcctgtccacagggatggccgggggtctggcaggaatgggaggcatccagaatgagaaggagaccatgcaaagcctgaacgacctcctggcctcctacctggacagagtgaggaacctggagaccgagaatgccggagagcaatatctgggagcacctggagaagagagaccccaggtcagagactggagccattacttcaagaccattgaggaagatctgagggctatcttcacaaatactgtggacaatgcccacatcgttctacagatcgacaatgcccgtcttgctgctgatgacttgagagtcaagtatgagacagagctggccatgtgccagtctgtggagagcgacatccgtgagctccgcaaggtcattgattacaccaatgtcactcagctgcagctggagatagagattgagctcaaggaggagctgctcttcatgaagaagaaccatgaagaggaagtaaaaggcgtacaagcccagattgccagctctgggttgactgtggaggtagatgcccccaaatctcaggacctcgccaagatcatggcagaaaactgggcccaatatggcaagctggctcggaagaaccgagaggagctggacaagtactggtctcagcagattcagaagagcaccacagtggtcaccacgcagcttgccgaggttggagctgctgagatgcttatggagctgagacatacagtacagtccttggagattctggactccatgagaaatctgaaggccatcttggagaacagcctgagggagatggaggcccgctacaccctgcagatggagcagctcaacaggatccggccgcacctggagtcagagctggcacagacccaggcagaaggacagggccaggcccaggagtatgaggccctgcagaacatcaaggtcaagctggaggctgagatcaccacctaccaccgcctgctggaagatggtgaggacttcaatcttggtgatgccctggacagcagtaactccatgcaaaccatccaaaagaccaccacctgccagatagtggatggcaaagtggtgtctgaaaacgagcaatgacaccaaagttctgagacattaagccagcagaagcagggtaccctttggggaacagaaggccaataaaaagttcagaggtcacacctta

SEQ ID NO: 37 - DNA, 4296 bp, Homo sapiens
  misc_feature: Incyte ID No 7925017CB1
ttatattaaaaactgtttcagtaaaaaggtttacagccatgcttttgtttaaagtatttctcctaagtatatactgtccatttaaaatattgggttggcgcattactttcaatgacaaccactgcaattacttaataaatcggttaagatggaggaatgccgagtcatatatccttcccccaaaattaaatatttgaagggatatgtcttattttatttctgggatagaatgtttttcattttcatgttactgaagtgtagttttactaaactactaaatcggagactgctaagcaccttccctgtgccaggcacagcactgggtagggtgagggccctgggagtaaatattctgggacaggcacagggggtgtacagggaaagagtctttcctctctgtggcatctgggaggctttaggatgtgggaaagatggtctcctcatctcagttgcccctcattttcaggcagacacgtatgaaatgattacactcccctgaaagatcagcttctacataattcaagatcatcctcaatctctctcatcagagaggaagaaaagccgcatgaattatttacagaacactaagttatttccttttatgagctccaactttgtttttgtctctgcaaactgagattttgtttatccatgaatagtttccccaggggcatgaaaaaatatgactcttaaattatgaatgaggttttttgtttcagtccatgttaactaaaacatgcaaaataaactatgtttttcattagatccctttcagctccctgcaaaaacagaaccaataaaagaacgagcagttcaaccagcacccaccaggaagcccactgtaattcgaattccagccaaaccaggaagtttacatgaggatccacaaagtccacctcctctccctgctgaaaaacctattggaaacactttcagtacagtatctggaaagctcagtaatgttgagagaactagaaacttggaatccaaccacccaggtcaaacaggaggttttgtgcgagtacccccaaggttgccaccgagacctgtgaatggaaaaaccattccaactcaacagcctccaaccaaggtgccccctgagagaccacctcccccaaagctttctgcaaccagaagatctaataagaaactgccttttaatcgatcctcttctgacatggatcttcagaaaaaacaaagtaacttggcaactggactctcaaaagccaagagtcaagtttttaaaaatcaagatccggtgctaccccctcgtcccaaaccaggacaccctctctacagtaaatacatgctgtctgtgcctcatggaattgccaatgaagatattgtctctcaaaaccccggagaactctcttgtaagcgtggggatgtacttgtgatgctgaagcagacggaaaataattacttggagtgccaaaagggagaagacactggcagagttcacctgtctcaaatgaagattatcactccacttgatgaacatcttagaagcagaccaaacgatccaagccacgctcagaagcctgttgacagtggtgctcctcatgctgtcgttcttcatgatttcccagcagagcaagttgatgatttgaacctcacttctggagaaattgtttatcttctggagaagatagatacagattggtacagagggaactgtagaaaccagattggcatatttcctgccaactatgtcaaagtgattattgatatcccagaaggaggaaatgggaaaagagaatgtgtttcatctcattgtgttaaaggctcaagatgtgttgctcggtttgaatatattggagagcagaaggatgagttgagtttctcagagggagaaattattattcttaaagagtatgtgaatgaggaatgggccagaggagaagttcgaggcagaactgggattttccccctgaactttgtggagcctgttgaggattatcccacctctggtgcaaatgttttaagcacaaaggtaccactgaaaaccaaaaaagaagattctggctcaaactctcaggttaacagtcttccggcagaatggtgtgaagctcttcacagttttacagcagagaccagtgatgacttatcattcaagaggggagaccggatccagattctggaacgtctggattctgactggtgcaggggcagactgcaggacagggaggggatcttcccagcagtgtttgtgaggccctgcccagctgaggcaaaaagtatgttggccatagtaccgaaggggaggaaggccaaagccttatatgatttccgaggggagaatgaagatgaactttccttcaaggctggagatataataacagagctggaatctgtagatgatgactggatgagtggagaacttatgggaaaatctggaatatttcccaaaaactacatacagtttctacagatcagctagaggagaagcttgtctgtgttccttggcacaagaactcacttgaactatcaccttgactatcagatatgtttttgcactattttttttaactgaaaaagaaatatctaagctgtacatggtacactagaattttctgaaagcagaaaacgttcagattttgtagttaattttcattacaatagaaaacatgcacatggaaacccatgagctaggattctaccgaggaaaacatctagtgggattagcaaggtgaagggaaagcatctggtggcatggcagcatggggaggctcacacacagaagttgcacgtggacatctgttttaatcagcacaagtgaattaaccatgcttcttcattttttttactttagttaaaaaagaggacatttaatattctacatgctgtaactatcaggacatggttagcaatctcaatttcatttttgatattcaaattaattcttacagcttgagcatatcagccttattaccagagcaaatccttccttcagatgggatagtttactgactagttggagcatttgtaagcacatggtgaaatcagcccctgcccaccaaaataatctttatgttaccaagtgattcccatttgtctaaggatttgaagggggtctaaattggatgtatcttacttagtctaaagaaccaaaaccatccctgaaatgccttgctaatacaactaatccttccatatatgtgccatacttatttttttcctcagtgtatactttatgttaacagggttattacaaagcacattttctgaatctgcaatcattcctttgacaattactggacccaaaggaaaattcattttctttgcattattccagtaatatataaaaactgtgtcttgttatagtagtacattatgaatcacatataaaatcttacaatacagaacaactgttaagatggaaaacagtgccaaacctccacagctcatttctttgtaatataatcagaatgaaaaataatttaagaggacagaagactggtacttttttgttttattttttctctagcttatccctgcacaattattagagtgaatgaaaaaccactttcctgctttccattgttataaattctaagcttaagataaaagtggttctttacatgactgaatcaattacaatttatgggctagagccaaataggttgaagacaatcatccaaacagatcaatggaatagaatttcattggaaatgtaaaacactttcccaacaatggtcatgactttcttctgtttttgagaagagtttcatatgctggaccacattttagcttttattgttttttttttcccattgtccaaaaagttaagcaacaagtggccacacttttacgtgactacaacctggagttctgcaaagaaggtaatatttacttggtctttgactaaagttatctccccattctatggttacattttattttggactatggggacttctaatacgttttggtaaagaagagagtataaagaaaattcttgtcaaatttcactcaaaagtaatttcatgagaaatcaatgatttaaagcattatccaaattaaattatcatttgcagcaaactgtacaacagcaggaaggatatggaatggaacatgaggtatatatctttgcctttataattttaacatcttatattgaagattctgaaaacctatctttattagaggaaaatctcaatcttcagttttggccttctgtcagcagaatgataagtgcaatagttgtaaatctacttgacactgtaataaactgaactgaactttcaaaaaaaaaaaaaaaaaaaaaaaaaaaaaaaaaaaaaaaaaaaaaaaagg

SEQ ID NO: 38 - DNA, 2045 bp, Homo sapiens
  misc_feature: Incyte ID No 6758789CB1
agatgaccttctccctctggcccctaacccctgcataatctgactcctgtgggtccccaggcaaaaggagacaacagaccgaccccgatgccccgtgcggctcgtgctggtgggtgcggggcccgctggcagggtcagctgtgcgtcctcacacgcctgctttgctcacctttgtgcctcacacatccacccttgaacgctgctcttctctctttcccaacagtgtcgcagccccaggctgctcccagcccgctggagaagtcgcccagcacggcgatcctgtgcaacacgtgtgggaatgtgtgcaagggcgaggtgctgcgggtgcaggacaagtacttccacatcaagtgcttcgtctgtaaagcatgtggctgcgacctggccgagggcggcttcttcgtgcggcagggcgagtacatctgcacgctggactaccagaggctctacggcacccgctgcttcagctgcgaccagttcattgagggtgaggtggtgtcggcgctgggcaagacctaccaccccgactgcttcgtgtgtgccgtctgccggctgcccttcccccccggggaccgagtgaccttcaacgggaaggaatgcatgtgccagaagtgttccctgcccgtatcggtgggcagcagcgcgcacctgtcccagggcctccgaagttgtgggggctgcggcacagaaatcaagaatggccaggccctggtagccttggacaagcactggcacttgggctgttttaagtgcaagagctgtgggaagctcctgaatgccgagtacatcagcaaggatgggctgccctactgcgaagctgactatcacgccaagttcggcatccgctgtgacagctgtgagaaatacatcacggggcgcgtgctggaggccggagagaagcactaccacccttcctgcgcgctatgtgtcaggtgcggccagatgtttgcagaaggcgaagagatgtatcttcaaggttcctccatctggcatccggcgtgtcgacaagcagccagaactgaagacagaaacaaggaaaccagaacttcctcagagagcatcatttctgtccctgcttccagcacctcagggtctccgagccgtgtgatttatgccaagcttggtggtgagatcctggactacagggacttggcagcccttcctaaaagtaaggccatctatgacatcgaccgccccgacatgatctcctactcaccctacatcagccactctgcaggggacaggcagagctacggcgagggggatcaggatgaccggtcctacaagcagtgtcggacctccagcccaagctccactgggtcggttagcctcgggcgctacactccgacctcacggtcaccacagcactacagccgtccagctggtactgtgagtgtgggtaccagtagctgcctctccctgtcccaacacccaagccctacatccgtgttcagacatcattacatcccctacttccgaggcagtgaaagtggccggagcacccccagcctctccgtgctctctgacagcaagccgcccccctccacctaccagcaggcacctcgccacttccacgtcccagacactggcgtaaaagataacatctataggaaaccccctatctacagacagcatgctgccaggcgatcggatggggaggatggaagcttggaccaggataacaggaagcagaagagcagctggctgatgctcaagggggatgcagacacaaggaccaattctccagacctggacacccagtccttgtcccacagcagcgggaccgacagagaccctctccaaaggatggcaggggacagctttcactcacaatacaagatctatccgtatgactccctcatcgtcacaaaccgaattcgcgtgaaactgcccaaagacgtggaccggacgagactggagagacacttgtcgcccgaggagttccaggaagtgtttgggatgagcatcgaggagtttgaccgcctggccctctggaagaggaatgaccttaagaagaaagcccttttgttctgacggctgccagc

SEQ ID NO: 39 - DNA, 1919 bp, Homo sapiens
  misc_feature: Incyte ID No 7488249CB1
gttccctgccacttcggtccgggcgcgccggtgggtttggcctgcgcggcggcggcggcgaggcgggggagcgagtgagcgcgaggggcgggcgcgagtgactgtgtgagtcacccgtacctggagtgcgagcgacgcagagccagcggcgcggagccggagccggagccgagacccagcgcctgcgagcccgagagcgcggccggccccaggcgccaggccccgtcgccctccccgtgcactcacccgtggcccggcgccgactccctacccggcgcccgccgcccgcagccctcccgcctgccaggaggcggtgcggggctcgccgggggaggtcacagcggctcctgggagccagcagccgccgccgccgccgcccccgggaaccgcgatcatgaacccccagtgcgcccgttgcggaaaagtcgtgtatcccaccgagaaagtcaactgcctggataagtattggcataaaggatgtttccattgtgaggtctgcaagatggcactcaacatgaacaactacaaaggctatgaaaagaagccctattgtaatgcacactacccgaagcagtccttcaccacggtggcagatacacctgaaaatcttcgcctgaagcagcaaagtgaattgcagagtcaggtcaagtacaaaagagattttgaagaaagcaaagggaggggcttcagcatcgtcacggacactcctgagctacagagactgaagaggactcaggagcaaatcagtaatgtaaaataccatgaagattttgaaaaaacaaaggggagaggctttactcccgtcgtggacgatcctgtgacagagagagtgaggaagaacacccaggtggtcagcgatgctgcctataaaggggtccaccctcacatcgtggagatggacaggagacctggaatcattgttgcacctgttcttcccggagcctatcagcaaagccattcccaaggctatggctacatgcaccagaccagtgtgtcatccatgagatcaatgcagcattcaccaaatctaaggacctaccgagccatgtacgattacagtgcccaggatgaagacgaggtctcctttagagacggcgactacatcgtcaacgtgcagcctattgacgatggctggatgtacggcacagtgcagagaacagggagaacaggaatgctcccagcgaattacattgagtttgttaattaattatttctccctgccctttgagctttattctaatgtatcccaaacctaatctttttaaaagatagaagatacttttaagacaacttggccattattttacaatgatgtatccttcctttgacaattagacacacaggtaccaggaagaaggaatgacctctgggctgaaaacagcagcattttcagtaattcctacaaacaaaaatctttgtgtctggacacctggtgctgctaattgtgttcatggtttcctttgattggctattgaacccttctgggaaatgtatttttgtagactttaatagagaagttgattgtcccttaaatgtagtgtgtgtttgaaacttcttagctgtcactttggaatcaccccaagccaattctcttaactctgtaatgcagccaataatttcaaacccgttttgcttttgagtcatgaggcaatttccaatattagtgaaaattgcccaatataataagtgtaaacagtggcagaaggacagtctggttaaaattatattgactggtggccttagggatctagaaacttctactaaacagagaaatttccttgcttccctaggctgactggtatctatttatttctcatttgtaccaaggccatctcctactctccatttatattctaatggacccaagtctatgctcagttcccagaatgtcgggccaattattccaggtctctgcgaag

SEQ ID NO: 40 - DNA, 5923 bp, Homo sapiens
  misc_feature: Incyte ID No 5046311CB1
cggcggcgtctgtggtttgaattccagcggcgccgccagagtctgaacaagagctggggtggagggggcggggacctggggagcccggcgggtcgctatcgcggggggtactagtggcgccgccgccacagacaccaacgccgtcgccacctctgtatccatgatggacttggtgttggaagaggacgtcaccgtccctgggacgctcagcggctgcagtggccttgttcccagtgtaccagatgacctggatggcatcaaccccaatgctgggttgggaaatggtctgctcccaaatgtgtcagaagaaacagtgtctcccaccagagcacggaacatgaaggactttgaaaatcaaatcactgaattgaagaaagaaaactttaacctaaagctccgcatctatttccttgaggaaagaatgcaacaggaatttcatggccccactgaacatatctacaaaactaacattgagctcaaggtggaagtagaaagtctgaagcgggaactccaggagagagagcagctgctcatcaaagcctccaaagcagttgagagcttagctgaagcaggtggctctgaaatccagcgggtgaaagaagatgctcgaaagaaggtgcagcaggtggaagatctcctaactaaaagaatactccttttggaaaaggatgtgacagccgcccaggcagaactggaaaaggcctttgcagggacagagacggagaaggctcttcggttgcgtttggaaagcaagctttcagagatgaagaagatgcacgagggggacttggcgatggctctggtcctggatgagaaagacagactgattgaggagttgaagctgtctttgaagagcaaagaagctttaattcagtgccttaaagaggagaaatctcagatggcatgtcctgatgagaatgtgtcatctggagagctccgaggactttgtgctgctccaagggaagaaaaggagagagaaactgaggctgcacaaatggagcatcagaaggagagaaacagctttcaagagaggatccaggcacttgaagaggacctgagagagaaggaaagagaaattgctacagagaagaaaaatagtctaaagagggataaagccattcagggtttaaccatggcattaaaatcaaaggaaaaaaaggttgaagaacttaactctgaaattgaaaagctcagtgctgcctttgctaaagccagagaggccctacagaaagcacagacccaggaatttcaggggtctgaagactatgagactgctctatcaggaaaggaagccctttcggctgcgctgcgctcacaaaacctcaccaagagtacagagaaccacagactgcgtagaagcattaagaagatcacccaggagctgagtgacttgcagcaggagagggagagactggagaaggacctggaggaagcccatcgagagaagagcaaaggagactgcaccatccgtgatcttagaaatgaagttgaaaaattacgcaatgaagtgaatgaaagagagaaagcaatggaaaatcgttacaagagtcttctgagtgaaagcaataaaaaattgcacaatcaagagcaagtgatcaaacatctaacagaaagtaccaatcagaaggacgtgttgcttcagaaattcaatgaaaaagatttggaagtaatacagcagaactgctatttaatggctgcagaggatcttgagctcaggagtgaaggcttaataacagaaaagtgctcttctcaacagccaccaggcagcaaaaccatcttctctaaggaaaagaaacaatcatcagactatgaagagctgattcaggtcttaaagaaagagcaggacatctatacccatctggtcaaatctctgcaggaatcagacagtatcaacaacctgcaggctgagttaaacaagatttttgccctgcggaagcaactggagcaggatgtgctttcatatcagaatttgcggaagaccttggaggagcagatcagcgaaattcggaggcgggaagaagaatcattttcactttatagtgatcaaacatcttatctaagtatttgccttgaagaaaacaatcggtttcaagtggaacatttttctcaagaagaacttaagaaaaaggtcagtgaccttatacagctagtgaaggagctgtatacagacaaccagcacctgaagaaaaccatttttgatctctcctgcatgggtttccagggaaatgggtttccagatagacttgcgtctacagaacaaacagagcttctggctagcaaggaggacgaggacacgatcaaaattggggaggatgacgagattaatttcctgagtgaccagcatttgcagcagagtaatgagattatgaaagacctttccaaaggaggctgcaaaaatggatacttaaggcacacggagtctaagatttcagattgtgatggggcccacgcacctggctgcctagaagaaggtgcattcataaacctgcttgcccctttgttcaatgagaaggccacattattactggaatccaggccagaccttctgaaagtggtacgggaactgcttctgggacaactattcttgacagagcaggaagtttctggagaacaccttgatggtaaaactgagaagacacctaagcaaaaaggtgaacttgtacattttgtccaaaccaactcattttccaagccacatgatgaactgaagttgtcttgtgaggcccagctagtaaaggcaggcgaagtgcccaaggtaggactgaaagatgcctcagtgcagactgtggccacggagggcgacctgctgagattcaagcatgaagcaacaagagaggcttgggaagagaaaccgatcaacactgcactcagcgcagagcatcggccagagaacctgcacggggtgcctgggtggcaggctgccctcctttccctccctggtattaccaacagagaggctaagaagtcccgcttgccaatcctaataaaaccatcccggtcattaggaaatatgtatcgtctccctgccacccaggaggtggtgacgcagctgcagagccagatcttggagctgcagggggagctgaaggagtttaaaacttgtaataagcaacttcaccaaaagttaattctggctgaagcagtgatggaggggaggccaacgcccgacaaaacgttgctgaatgctcagccccctgtgggagcagcctaccaggacagcccaggagagcagaaaggaattaaaaccacatcttctgtctggagagacaaggaaatggacagtgatcagcaaagaagctacgagattgactctgagatttgcccacctgatgaccttgccagcttgccatcatgcaaagaaaatcctgaagatgttctgagcccaacttcagtagctacttacctgagttccaagagtcagccttctgctaaagtcagtgtgatggggactgatcagtcagagagcattaatacctcaaatgagacagaatacttaaaacagaaaatccatgacttggaaactgagctggaaggctaccagaatttcatatttcagcttcaaaagcactcccagtgcagtgaggccataattacagttttgtgtgggacagaaggggcccaggatggcttgagcaagcccaagaatggttctgatggggaagaaatgaccttttcaagtttgcaccaagtgcgatacgtgaaacacgtgaaaatcctcggtccgctggccccagagatgattgacagcagggtgctggagaacctcaaacagcagctggaggaacaggaatacaagctgcagaaggagcagaatttgaacatgcaacttttcagtgagatccataatctgcagaataagttcagagatctctcacctcccagatacgattcattagttcagtcccaagccagggagctctcccttcaacggcagcagattaaggatggccatggcatctgtgtcatctcccgtcaacacatgaacaccatgattaaggcatttgaggagttgctgcaggccagtgatgtggattactgtgtggccgagggtttccaggaacagctgaatcaatgtgctgagctgctggagaaattggaaaagctatttctcaacggaaaatcagttggagtggaaatgaacacccagaatgaactgatggagaggattgaggaagacaacttaacctaccaacatcttctgcctgaatctcctgagccttcagcctctcatgcgctctctgattatgaaacatctgaaaagtccttcttctcacgagaccagaagcaagataatgagacagagaagacttcagttatggtgaacagtttttctcaagacttactaatggaacacatacaggaaattcgaactttgagaaagcgtttagaagaatctattaaaacaaatgagaagctacggaaacagttggaacggcaaggatctgaatttgttcaaggttctacaagcatttttgcttctggttcagagcttcatagttctctaacatcagaaattcatttcttgaggaagcagaaccaggccctcaatgcaatgctcattaaaggatccagagataaacagaaggagaatgacaaattacgagagtccctctccaggaagaccgtgagcctggagcaccttcagcgggagtatgccagcgtgaaggaagaaaatgaaaggctgcagaaagaaggcagcgagaaggagagacacaaccagcagctgatccaggaggtccgctgcagcggccaggagctgagcagggtgcaggaggagctgaagttgaggcagcagctgctctcacagaatgacaagctattgcagtctctccgagtggagctgaaggcgtatgagaagctggatgaagagcacaggagactgagagaggcgtcgggagaaggctggaaggggcaggatcctttcagggacctgcacagcctcctgatggagatccaggctctgcgcttgcaactagaaaggagcatcgaaaccagcagcactctgcagagcaggctcaaggaacagctggcaaggggggcagagaaggcacaggaaggagccctcactctggctgtccaagccgtgtccatccctgaggtgccccttcagcctgacaaacacgatggtgacaaatatcccatggaaagtgataattcatttgatctgtttgattcctcccaggcagtgacaccaaaatcagtttcagagactcctccactctctgggaatgacacggactccctctcctgcgacagtggcagttcggcaactagcactccgtgtgtgtcccgcctggtcactggccaccacctgtgggccagcaagaatggccgccatgtcctgggcctgattgaggactatgaggccctgctcaaacagatcagccagggacagaggctccttgctgaaatggacattcaaacccaagaggctcccagctccacaagtcaagagctgggaacaaagggtccacacccagcaccactgagcaagtttgtgagcagtgtgagcacggccaagctgaccctggaagaggcctacaggcggctgaagcttctctggagagtctcactccccgaggatggccagtgcccccttcactgtgagcagattggagaaatgaaggcagaggtcaccaaactacataaaaaattgtttgaacaagaaaagaagttgcaaaacaccatgaagcttttgcagctgagcaagcgccaggaaaaagtcatctttgatcaattggtcgtaacccacaaaatccttcggaaggccagaggaaacctggagcttaggcctgggggagcccatccaggaacatgcagtcccagcagaccaggctcctgagaagaactttcagccaataaagcttgtgcttcccccaccgagctcacgctgtctctttgttccaagtgtggttcctattta

SEQ ID NO: 41 - DNA, 2789 bp, Homo sapiens
  misc_feature: Incyte ID No 931056CB1
gttcgtctgctgggtttgcggagcagctagctactcggcgggatctcccggcaggatgggtaaaaagataaagaaggaagtagagcctcctcctaaggatgtgtttgacccattaatgattgaaagcaaaaaagcagcaactgtggtgttaatgcttaattctccagaagaggaaattttggctaaagcatgtgaagccatttataaatttgctttaaaaggtgaggaaaataaaacaaccctccttgaacttggagctgtggaacctttaactaagctactcacccatgaagacaaaattgtaagaagaaatgctactatgatatttggaatcctggcttctaataatgatgttaaaaaattgttaagggagttagatgtcatgaattctgtcattgcccagctcgctccagaagaagaagtagttatccatgagtttgctagtctttgtctagcaaacatgtctgcagagtacaccagtaaagtgcaaatatttgaacatgggggattagagccactcatcagactactgagtagccctgacccggatgtaaagaagaactctatggaatgcatttacaacttggtgcaggattttcagtgtcgagctaaacttcaagaactaaatgcaatacctcctatcttagatctcttgaagtcagaatatccagtgattcagttgttggctctcaaaaccttaggtgttattgcaaatgataaggagtctcgaacaatgctaagagacaatcaaggattggaccatcttattaagatcctagaaactaaggaattgaatgaccttcatatagaagcacttgcagtgatagccaattgccttgaagacatggatactatggtgcagattcagcagacagggggtcttaaaaagctcctgtcatttgcagaaaactctacaattcctgatattcagaagaatgcagcaaaagccattactaaagcagcttatgatcctgaaaatagaaaactttttcatgaacaagaggttgaaaagtgccttgtagcccttttgggttctgaaaatgatggaactaaaattgctgcttcccaagctatttcagcaatgtgtgagaattcaggcagcaaagattttttcaataatcaggggattccacagttaattcagttgctaaaaagtgacaatgaagaggtacgggaagcagcagctctagccctggcaaacctaaccacttgcaaccctgctaatgcaaacgctgctgctgaagctgatggtattgatccattaataaacctcctgtctagtaaacgagatggagccattgccaacgctgctacagtattaacaaacatggccatgcaggagcccctgcgcctgaacatacagaatcacgacatcatgcatgccatcatcagcccactgcgttctgcaaacacagtcgtgcagagcaaagctgctctcgctgtcaccgcaactgcgtgtgacgttgaagcccggactgagttaagaaattctggtggattggagcccctggtagagctgctacgctccaagaatgatgaagtgaggaagcacgccagttgggcagtgatggtctgtgctggtgacgagctgacggccaatgaattatgcaggctcggggctttagatatccttgaagaagttaacgtatcaggaactcggaaaaataaattcagtgaggcagcttataataagttgctcaataacaatctttccctgaaatacagccagactggctatttgtcatcaagtaacataattaacgatggattctatgattatggtcggataaatcccggcaccaaactgttgcctttgaaggagctctgcttacaagaaccaagtgacctacgggctgtactcttaatcaacagtaaatcttacgtttctccaccttcatctatggaagataaatcagatgttggttatggacgaagtatttcttcttcatcttccttaagaagatcaagtaaagaaaagaataattatcattttagtgctggatttggatctcccatagaagacaaatcagagccagcttctggacgaaatactgttctcagcaaaagcgccaccaaagaaaaaggatggaggaaaagcaaaggaaaaaaagaagaggaaaaagtgaaagaggaggaagaggttatggtggtaccaaaatttgttggtgaaggaagctctgacaaagaatggtgtcctccctctgaccctgatttctctatgtatgtgtatgaggtgaccaaatcaatactgccaataaccaatattaaggaacagattgaggatctggcaaagtatgtagcagaaaaaatgggtggtaagattccaaaagagaaactacctgatttcagctgggaacttcacataagtgaactgaaatttcaacttaaatccaatgttataccgattggacatgtcaaaaaaggaatcttctaccatcgagctttgcttttcaaggctctggctgatagaattggcattggttgctccctagttcgcggagagtacggtagagcgtggaatgaagtcatgctgcagaatgactctcggaagggagtgattgggggcctccccgctcctgagatgtacgtgattgacctcatgttccatccaggtggactgatgaagttgagaagtcgagaggctgatctttacagattcatttaagccatcagacgaacacaagagaggctcaaacaagaaattcactgtgtacactctctaagacattctccaaattgattttatctctttaaataaaaactttaaataaaaaaaaaaaaaaaaaaaa

SEQ ID NO: 42 - DNA, 4077 bp, Homo sapiens
  misc_feature: Incyte ID No 2578937CB1
cgcgagcaggcgctgccaaccccacttctgcccgggattccaatctgaggagcaggaggaccggggcgccggtgtcctgccgcctccttctccttgctctcacctgcgcctattagtccacgcgccttcaaggccaggggctacagcccagacagagaggggacagcagagggagagagagcacctgaggatacagagctggcactggactgccttttcaccccccaggtgatgagtgaggttcgaagaacggaagatttaaaaagcagccggggcctccgtattgaatgaaagacccagtgcaaagacatcaccatgaacactagcattccttatcagcagaatccttacaatccacggggcagctccaatgtcatccagtgctaccgctgtggagacacctgcaaaggggaagtggtccgcgtgcacaacaaccacttccacatcagatgcttcacctgtcaagtatgtggctgtggcctggcccagtcaggcttcttcttcaagaaccaggagtacatctgcacccaggactaccagcaactctatggcacccgctgtgacagctgccgggacttcatcacaggcgaagtcatctcggccctgggccgcacttaccaccccaagtgcttcgtgtgcagcttgtgcaggaagcctttccccattggagacaaggtgaccttcagcggtaaagaatgtgtgtgccaaacgtgctcccagtccatggccagcagtaagcccatcaagattcgtggaccaagccactgtgccgggtgcaaggaggagatcaagcacggccagtcactcctggctctggacaagcagtggcacgtcagctgcttcaagtgccagacctgcagcgtcatcctcaccggggagtatatcagcaaggatggtgttccatactgtgagtccgactaccatgcccagtttggcattaaatgtgagacttgtgaccgatacatcagtggcagagtcttggaggcaggagggaagcactaccacccaacctgtgccaggtgtgtacgctgccaccagatgttcaccgaaggagaggaaatgtacctcacaggttccgaggtttggcaccccatctgcaaacaggcagcccgggcagagaagaagttaaagcatagacggacatctgaaacctccatctcaccccctggatccagcattgggtcacccaaccgagtcatctgcgacatctacgagaacctggacctccggcagagacgggcctccagcccggggtacatagactcccccacctacagccggcagggcatgtcccccaccttctcccgctcacctcaccactactaccgctctggtgatttgtctacagcaaccaagagcaaaacaagtgaagacatcagccagacctccaagtacagtcccatctactcgccagacccctactatgcttcggagtctgagtactggacctaccatgggtcccccaaagtgccccgagccagaaggttctcgtctggaggagaggaggatgattttgaccgcagcatgcacaagctccaaagtggaattggccggctgattctgaaggaagaaatgaaggcccggtcgagctcctatgcagatccctggacccctccccggagctccaccagcagccgggaagccctgcacacagctggctatgagatgtccctcaatggctcccctcggtcgcactacctggctgacagtgatcctctcatctccaaatctgcctccctgcctgcctaccgaagaaatgggctgcacaggacacccagcgcagacctcttccactacgacagcatgaacgcagtcaactggggcatgcgagagtacaagatctacccttatgaactgctgctggtgactacaagaggaagaaaccgactgcccaaggatgtagacaggacccgtttagagcgccacctgtcccaggaagagttctaccaagtctttggcatgaccatctctgagtttgaccggctggccctctggaagaggaatgaactgaagaagcaagcccggctgttctaggcagaggctctataaatatatatgcatttatataaagatatatgtaaaatctctctactgaagctcggtataatcctctcttgtgtaatgggacacactgcctgccatgagacttgcttttctgtactgtcaggcaagcccacgtcatcgagatatttttatgctccttactttctcttttctaagtgctgtgggatctgggaagggatttgaggggactctgtccttttattggggatcctttttatactgaaacatctgtcctaacttgagtgccccaaggtccaactctctttcctaaagaaggtgcctgaagaagtctctcttctctctgcttcgtggcccctttcttaaatttctagggctgatgctgaccatgtggtttccacaccttattggccccagaggggccctcccatgggaagatctgcagcagtctccccaaatcagtgagcacctttgagcgcccacgaagaactttctcaacacccccaattaggagctcagtgctctcttggggcaatgcaattaaaagggtgagcctcaaatctagtcattacaccagtcaacagaagtggacagggcctaggcctctcctcagctccttaaccctcctccttctgccctggattgtaacctctcccttgtccaaatctaggattcctggtaggaaaaggaaaaggcccttcccttccctccaccacttccaactggcccctttgcctgacctggacttggagaaccagaggaaaagagagggagcggaagtgggagatggagcagggcacctgttagaatcagagctgcaggatttcttgggaccctcctctctccctcactgctcccagcacctcctgacccttccctctttcaaggagaagcccatgattgcagcttgtattctttagccttattacaatctatgtgcctgacaactcaacacaccgcagggctaatgttcccaccagagctccaactgaacaaccagacagacaactctcatcatcctccagagagaaaataggccgtgtctcaaagaaaggttcttggtctatgcctctggtctgtgggctggcagggcaaccataccatacccccgccagtcctcggctcctgctgcaaagttggccatgtttcacagggaaacttttggaagagtggctgcttatgagattccaaaatgaagtgttggccaacaccgctcatggccatcctggattttcccagtggcttcccttcctgctcgcctccctgaacaggggagaaagcttaacctctcttctcctctccaaacctttcaccttgaatgggtaatgtttggtgggggctgttccttcttggagaagccttgagtcggaccattttgagatcatggaggaaggatgaagaagtgaaaatgacaataatgactctcaagaggctggcgatgtgacatggcaaatgtagaactgacttaaattgaacaaaccctcactgagcacctctgatgttgagcacctgctgaatactgagcactgaatgggggagggggaggggagcacggggtgagtcaacctgggactcggtctcagggatatgcctaccaatagcgggtatcgtaaggcatgtacccaaacataacggatgtaaggcagaaagtgatcggagaaggaatgagaaagtgtgcgtgatgttaatgaaaagtcatatgcagctagagcagacccaggaaagctttctggaagagattgcatctgaggaaattcaggaaggatctttgtagattggggggagattctaaattgaaggggtgatagggtgaggggccagagggaagtctgctgtgttctcatgtaggatgtcagccctccctgcaacttctctttttggccaatgtcttttcactttcctgaccctttagaatcatccccagccagacgcaatcatggaagttgccttattgtcactggttaagaacttggcgagattgaagggcttttgttattgttgttggatatttttgtttcccataaaagcacatcatttcaaccctaaaaaaaaaaa

SEQ ID NO: 43 - DNA, 4935 bp, Homo sapiens
  misc_feature: Incyte ID No 489786CB1
cggacgcgtgggggaggagcagggggagggctgtcaaattcgggagccagattttttcccttctcctggcaatcccttccgcttccccggctcccgacgtgacatctgcgggccggggacctgcatgtgtgtgcgcgcgaaggagcggaagaatggcagtgctcaaactcaccgaccagccaccattggttcaggcaatcttcagcggtgatccagaggagatccggatgctcatccataaaactgaagatgtgaatactctggattctgagaaacgaacccctcttcatgtggccgcatttctgggagatgcagagatcattgaactcctgattttgtcaggagctcgtgtaaatgccaaggacaacatgtggctgactccactgcaccgggctgttgcttccagaagtgaagaagcagtacaggttttgattaagcactcagctgatgtcaatgcaagggacaagaactggcagacccctcttcatgtggcagcagccaacaaggctgtcaaatgtgcagaagtgatcattcccctgctgagcagtgtcaatgtctccgaccgaggggggcgcacagccttgcaccatgcggctctgaacggccacgtggagatggtcaatttactcttggccaaaggggcaaatatcaatgcatttgacaagaaggaccggcgtgctctgcactgggcagcatacatgggccacttggatgttgtagcattgctcattaaccatggcgcagaagtgacctgtaaggataagaagggttatacccctctgcatgctgcagcctccaatggacagattaatgttgtcaagcatctcctgaacctgggggtggagattgatgaaatcaatgtctatggaaatacagcgcttcacatcgcctgctacaatggacaggatgctgtggttaacgagttgattgactacggtgctaacgtgaaccagccaaacaataatgggttcacccctttgcattttgctgctgcctccactcatggtgctttgtgtcttgaattgttagtaaacaacggggcagatgttaacattcagagtaaagatggcaaaagtccactgcacatgacagctgtccatggaaggttcacacggtcacagaccctcattcagaatggaggtgaaattgactgtgtggataaggacggcaacactcctctccatgtggctgcaagatacggtcatgagcttttgattaacaccttaataaccagcggagctgacacagccaagtgtggaatccatagcatgttccctttacatttagctgccctaaatgctcactctgactgctgcagaaagttgttatcatcgggctttgaaatagacaccccagataaatttggaagaacgtgccttcatgctgctgctgcaggaggtaatgtggaatgtataaaactcttgcagagcagcggagcagatttccataaaaaggacaagtgtgggaggacccctttgcactatgcagctgcgaattgtcatttccactgtattgagacattagtgaccacaggggccaacgttaatgaaacagatgactggggacgcacagctttgcattacgccgctgcatcagacatggatagaaataagactatcttaggaaatgcccatgataattcagaagaacttgaaagagccagggaactgaaggaaaaggaagccacactatgtctagagtttctgcttcaaaatgatgcaaatccatctatccgggacaaggaaggttacaatagcatacattatgctgccgcctatgggcacaggcagtgtctggaattgcttttggaaagaacaaacagtggatttgaagaatcagattctggtgctactaagagtccactccacttagctgcctacaatgggcaccatcaagccttggaagtccttctgcagtcgttggtggacctggacatcagggatgagaaaggccgcactgctctggatctggctgcctttaaaggacacacagaatgtgtggaagcgcttatcaatcagggcgcatccatctttgtgaaagacaatgtaaccaaaagaaccccacttcatgcctcagtaattaatggtcacacactgtgtttacggctgttgctagaaattgcagacaacccggaggcggtcgatgtgaaagatgccaaaggacaaacaccactgatgcttgcagtagcatatggacatattgacgctgtttcattgttacttgaaaaggaagccaacgtagacactgttgacatcctaggatgcacagctttacacagagggattatgacaggacacgaggaatgtgtgcaaatgctgctggaacaagaagtgtcaattctctgtaaagattccagagggaggacgcccttgcactatgcagctgctcgtggccacgccacgtggctgagcgagctgctccaaatggctctttctgaggaggactgttgtttcaaagataaccaaggctacacgccgctgcactgggcttgttacaatggtaatgaaaactgtatagaggtacttttggagcaaaaatgttttcgcaaatttatcggtaatccctttactccactgcactgtgcaataatcaatgatcatgggaattgtgcatcattgctgcttggggccatagattccagtatcgtcagttgtagagatgacaaaggcaggacaccccttcatgcggcagcatttgctgatcatgtggagtgcttgcagcttcttctgagacacagtgctccagtgaacgcagtagataattcagggaaaacagcactgatgatggctgctgagaatgggcaggcaggcgctgtggatattttggtgaacagtgcccaggctgatctgactgtaaaggataaggacttgaatacacccttacatttggcttgtagtaaaggtcatgaaaaatgtgccttgttaatacttgacaagatacaagacgagagccttattaatgaaaaaaataatgcactgcagacacccctccacgtcgctgcgcgcaatggcttaaaggtgttagttgaggagttgctggccaaaggggcctgtgtacttgctgtagatgaaaatggtcataccccggccctggcttgcgctcctaacaaagacgtggctgactgcctggccctcattttggctaccatgatgcctttttctccttccagtacaatgatggctgtcaacttcgtttgtttaaaaaaagacaatttgagcaggacgaccctctccaatctgggtagcatggttagcctgtgcagtaacaacgtaggctcggaggatgggtacaatgaaaatgattctgattcggaaacgttttgactttggactgtagaagcttttctttgatcacctgtgttggaggaaaggaaagaagcttgaattccaggtttatgttgtattcaagtattatttggggcctgggcttccagaagctagtagagaaggaattaatcgagggagggcagtgaggctgttgggtggaacagtcacacagatggccccagttttgttttgtttcacattttctttaaatctaagatacttctgtgaaagtctacctctcattttaagtaaagaataaaagatgtatttaattcctgttctttggggataatgcagcagagaggcagctgttcgctatgaataatttttcttttcatgtatttagcagtgagttctcagaagtatatcagtgtgacattcatgtcccctgggagggaagggaagaggcagataggaaatgcctcaaacttcttctcactttgatgtttacttcctcactagaagccaaaggtaaaggtgtccatcttcagaaataatgcctgtaataatctttttagagagtccaactatttatatcctctctatagtaagcatttgaatatccagaacttctttctgaggatctctgttaaagttgccagatgaattgaaaaattgaacttgctttttttgtttaacacagtgttccctataacaagcctgtgactaattttcacttaagtgattgaacccaaatttatctaataacatttcaggtgaaaattatattgcaccaaaactttgacacaatatgcaaaaatagtatgaaactaccctttgttaatttattcttaaatcaaaatactaacttttaaaaaacatcagaatcatcagcattgttcatcagtagtaaaatgagcttctcagtcaaggtcataccaagtcagtgaaaagtgtgactgcaaaaaggaacaaaaaaagtgaaagcagaaagtagaaatgggtgatttagcatgtaaaaccgattgccagttgcttgatgatgaaatcaacatagtgaatactgtgtctgctccctctgccaaagcttctaggtcaaatggaccccgttccacacctggaaccgctgtacaaaaagaagaatgagactctttaaaaattatgcacatacacatgcacacatatatgtgtgcgtgtgtatatatatatatatgtgtgtgtgtgtgtgtagttcatcagccagctacacatgaggaccaaaatgcttcagtctaaaatggaagatacacatttttttccttcaaaatgcaagtgagaactgaagtagcttttttatggagttaaatgtaatctttctgtgtaccagtctttgttgtattttatatttcttaggacacagatttctagttgaccacttaacatttgtaactgatgatgtgttgaccttttttttttttttttgccaaactagagaaaatgtccatatacttttgctgtaaatgtgtttatatttatttgaaatgaaacaaatggtgaggaaacatccattatttgttctctattttaattgctatgtatcttatttagaataacaaaaaaaaagtgaaaaaaaaaacacttgaccctgggctacccaaaagcccaagggccaaacctagcatttccactg

SEQ ID NO: 44 - DNA, 3400 bp, Homo sapiens
  misc_feature: Incyte ID No 2240034CB1
gcttactaccccagggcgaacggacggacgacggaggcgggagccggtagccgagccgggcgacctagagaacgagcgggtcaggctcagcgtcggccactctgtcggtccgctgaatgaagtgcccgcccctctgagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtactgggacattccaaatggctgcaaactaatcaggaatcgatcggattgtaaggacattgattggacgacatatacctgtgtgctaggatttcaagtatttggtgtctggccagaaggatctgatgggacagatatcaatgcactggtgcgatcccacaatagaaaggtgatagctgttgccgatgacttttgtaaagtccatctgtttcagtatccctgctccaaagcaaaggctcccagtcacaagtacagtgcccacagcagccatgtcaccaatgtcagttttactcacaatgacagtcacctgatatcaactggtggaaaagacatgagcatcattcagtggaaacttgtggaaaagttatctttgcctcagaatgagactgtagcggatactactctaaccaaagcccccgtctcttccactgaaagtgtcatccaatctaatactcccacaccgcctccttctcagcccttaaatgagacagctgaagaggaaagtagaataagcagttctcccacacttctggagaacagcctggaacaaactgtggagccaagtgaagaccacagcgaggaggagagtgaagagggcagcggagaccttggtgagcctctttatgaagagccatgcaacgagataagcaaggagcaggccaaagccacccttctggaggaccagcaagacccttcgccctcgtcctaacaccctggcttcagtgcaactcttttccttcagctgcatgtgattttgtgataaagttcaggtaacaggatgggcagtgatggagaatcactgttgattgagattttggtttccatgtgatttgttttcttcaatagtcttattttcagtctctcaaatacagccaacttaaagttttagtttggtgtttattgaaaattaaccaaacttaatactaggagaagactgaatcattaatgatgtctcacaaattactgtgtacctaagtggtgtgatgtaaatactggaaacaaaaacagcagttgcattgattttgaaaacaaacccccttgttatctgaacatgttttcttcaggaacaaccagaggtatcacaaacactgttactcatctactggctcagactgtactacttttttttttttttccctgaaaaagaacccggaaaaagtgtccccttctgg

SEQ ID NO: 45 - DNA, 981 bp, Homo sapiens
  misc_feature: Incyte ID No 3438037CB1
aggaggcggcatgagcagcgcgcgacagagctgacgccgcgcccccgccggccccatgtccttcgccacgctgcgcccggcgccgccgggccgctacctgtaccccgaggtgagcccgctgtcggaggacgaggaccgcggcagcgacagctcgggctccgacgagaaaccctgtcgcgtgcacgcggcgcgctgcggcctccagggcgcccggcggagggcggggggccggcgggccgggggcggggggccagggggccggccaggccgtgagccccggcagcggcacacggcgaacgcgcgcgagcgagaccgcaccaacagcgtgaacacggccttcacggcgctgcgcacgctgatccccaccgagcccgccgaccgcaagctctccaagattgagacgctgcgcctggcctccagctacatctcgcacctgggcaacgtgctgctggcgggcgaggcctgcggcgacggacagccctgccactccgggcccgccttcttccacgcggcgcgcgccggcagccccccgccgccgcccccgccgcctcccgcccgcgacggcgagaacacccagcccaaacagatctgcaccttctgcctcagcaaccagagaaagttgagcaaggaccgcgacagaaagacagcgattcgcagttaggaggtggccggcagcagccaggaggcagacgctgctgggggaggtggacgcccggggtgactgcagacagcccccaccttggacctgagctgggcaaggcccaccgcaagcatgcccccaggccagccctggctgcgagcggggccgagggacagacggacgtacagacaggcgccggcagcgggactctgcgctggccccagcacctgcccgggcccactggaactttctgcgctggcttttcttccggcccactgtgtgatggcatcttgtgtttttgatatgataatataaagtctgaaaattttgtataattaaaa

SEQ ID NO: 46 - DNA, 3097 bp, Homo sapiens
  misc_feature: Incyte ID No 6578021CB1
tagctcacatagggaatttggccccgagcggtaattcggcagaggctgagacggcagcaggaagggagctttgccaggtttctcgcctgccctgctagaaggaggcccttgatcctttaccaccgtctcctcccctctggacgagcattcaagctgggctgcctgaaagggcagtcctaccggacacatgtaaagttgtggggagacagaatcccctctacgctttcttcaggtgatgggtacattctgagtacctcttgcccttttcccacgtgacggccctgctcgggaggccctccagtctctgagccagtggcttcgggtgcaggagcaggagatggaactggtaaaggcagccctggcagaagcccttcgcctgctgcggctgcaggtgcccccttcctccctgcagggctctggcacaccagctcctccgggggacagtcttgcagcccccccgggactgccacccacgtgcaccccttccttggtgagccgaggcacccagacggagacagaggtggagctcaagtcatcccctggaccccctggcctgagcaatggacccccagcccctcaggggggccagcgaagagcctagtgggacccaatctgaaggagggggcagcagcagcagtggtgctggctcccctggccccccggggatcctcaggcccttgcagcccccacagcgtgctgacacgccgcgaagaaattcttcctcctcctcatccccctcagagtggcctcggcagaagctctccaggaaggcaatctcctccgccaacctgttagtgcggtccgggagcacagagagccgtgggggaaaagaccccctctccagccctgggggccctggatctcggaggagcaattacaatttggaaggcatctcagtgaagatgttccttcgagggcgccccattaccatgtacatcccgtctggcatccgcagccttgaggagctgccgagtggcccaccgccagagaccctcagccttgactgggtttatgggtacaggggtcgtgactcccgctctaatctgtttgtgttgcgctctggggaggtggtctactttatcgcctgtgtggtggtgctgtaccggcctggaggaggcccagggggtcctggaggtggcggccagagacattaccgggggcacacagactgcgttcgatgccttgctgttcaccctgatggtgttcgggtagcctcgggacagacagctggagtggataaggatggaaagcccctgcagcctgtggttcacatctgggactcagagacgctgttgaaactgcaggagattggactgggggccttcgagcggggtgttggggccctggccttttcagctgcggatcagggtgcctttctttgtgtggtggatgattccaatgagcacatgctgtcggtgtgggactgcagccggggaatgaagctggctgagatcaagagtacaaatgactcagtcctggccgttggcttcaaccctcgtgacagcagctgcatcgtcaccagtgggaaatctcacgtccacttctggaattggagtggtggagtaggggttcctgggaatgggacccttacccggaaacagggtgtctttgggaaatacaagaaacccaagtttatcccttgctttgtgttccttccggatggagacattctcactggagactcagaggggaacattctcacctgggggcggagcccttcagattccaagaccccaggcaggggtggcgccaaagagacctatgggattgtggcccaggctcacgctcatgaaggttctatcttcgccttgtgtctccggagggacgggacagtgctgagtggtggcgggcgggaccgccggctggtacagtgggggcccgggttggtggccctccaggaggctgagattcccgagcacttcggggccgtgcgagccattgctgaagggcttggctctgagctgctggtgggaaccacgaagaatgcattgctgaggggagacctggcccagggcttctcccctgtaatccagggccacactgatgagctctgggggctctgcacacacccctcccagaaccgcttcctcacctgcggccacgaccggcagctctgcctgtgggatggggagagccatgcactggcctggagcatcgacctcaaggagactggtctctgtgctgacttccacccgagtggggcagttgtggccgtaggactgaacacggggaggtggttggttttggacacagagaccagagagatcgtgtctgatgtcattgatggcaatgagcagctctcagtggtccggtacagcccagatgggttgtacctggccattggttcccatgacaacgtgatctacatctatagtgtttccagtgatggtgccaaatccagccgctttggccgctgtatgggtcactccagcttcatcactcatcttgactggtccaaggatgggaatttcatcatgtccaattctggggactatgagattctttactgggacgtggctggaggctgcaagcagctgaagaatcgctatgagagccgagaccgggaatgggctacctacacctgtgtgctgggctttcacgtctacggcgtctggccggacggctccgatgggaccgacatcaactccctgtgccgctcccacaacgagcgcgtggtggcggtggccgacgacttctgcaaagtgcatctcttccagtacccgtgcgctcgtgccaaggcgccgagccgcatgtacgggggccacggcagccacgtgaccagcgtccgattcacgcacgacgactcgcacctcgtctcgctgggcggcaaggacgccagcatcttccagtggcgagtgctgggcgctgggggcgcggggccggcgcccgccacgccctctcgaaccccctccctgtcccccgcctcctccctcgacgtttgatcgctgcctggcgggaccgactggcccggcggcgtggccccgccccgccctgcccttccctggcccaatcccccacgactaggggccgactctttcctggactgacttcgagacattcccgatcgcgcattttcctggagggcgcgaacggcgcc

SEQ ID NO: 47 - DNA, 3472 bp, Homo sapiens
  misc_feature: Incyte ID No 8013295CB1
ggagctcttctcactcaagcccgagtttctatgttcagacatagtacattcatcactgtgtccttccaggatttggaagtctgacaaaacaccattccagtagctgcatctccaggttttgagtctagaaatgaatttaagatgtgatctcttggataagaaagctaaccccaatgccaaagccctgaatggctttacccctcttcatattgcctgcaagaagaatcgaattaaagtaatggaactccttctgaaacacggtgcatccatccaagctgtaaccgagtcgggccttaccccaatccatgttgctgccttcatggggcatgtaaatattgtatcacaactaatgcatcatggagcctcaccaaacaccaccaatgtgagaggagaaacagcactgcacatggcagctcgctccggccaagctgaagttgtgcggtatctggtacaagacggagctcaggtagaagctaaagctaaggatgaccaaacaccactccacatttcagcccgactggggaaagcagacatagtacaacagctgttgcagcaaggggcatctccaaatgcagccacaacttctgggtacaccccacttcacctttccgcccgagaggggcatgaggatgtggccgcgttccttttggatcatggagcgtctttatctataacaacaaagaaaggatttactcctcttcatgtggcagcaaaatatggaaagcttgaagtcgccaatctcctgctacagaaaagtgcatctccagatgctgctgggaagagcgggctaacaccactgcatgtagctgcacattacgataatcagaaagtggcccttctgcttttggaccaaggagcctcacctcacgcagccgcaaagaatggttatacgccactgcacatcgctgccaaaaagaaccagatggacatagcgacaactctgctggaatatggtgctgatgccaacgcagttacccggcaaggaattgcttccgtccatctcgcagctcaggaagggcacgtggacatggtgtcgctgctcctcggtagaaatgcgaatgtgaacctgagcaataagagcggcctgaccccactccatttggctgctcaagaagatcgagtgaatgtggcagaagtcctcgtaaaccaaggggctcatgtggacgcccagacaaagatgggatacacaccactgcatgtgggctgccactatggaaatatcaagattgttaatttcctgctccagcattctgcaaaagttaatgccaaaacaaagaatgggtatacgccattacatcaagcagcacagcaggggcatacgcatataataaatgtcttacttcagaacaacgcctcccccaatgaactcactgtgaatgggaatactgcccttggcattgcccggcgcctcggctacatctcagtagtggacaccctgaagatagtgaccgaagagaccatgaccacaactactgtcacagagaagcacaaaatgaatgttccagaaacgatgaatgaagttcttgatatgtctgatgatgaaggtgaagatgcaatgaccggggacacagacaaatatcttgggccacaggaccttaaggaattgggtgatgattccctgcctgcagagggttacatgggctttagtctcggagcgcgttctgccagcctccgctccttcagttcggataggtcttacaccttgaacagaagctcctatgcacgggacagcatgatgattgaagaactccttgtgccatccaaagagcagcatctaacattcacaagggaatttgattcagattctcttagacattacagctgggctgcagacaccttagacaatgtcaatcttgtttcaagccccattcattctgggtttctggttagctttatggtggacgcgagagggggctccatgagaggaagccgtcatcacgggatgagaatcatcattcctccacgcaagtgtacggcccccactcgaatcacctgccgtttggtaaagagacataaactggccaacccacccccacatggtgaaaggagagggattagcagtaggctggtagaaatgggtcctgcaggggcacaatttttaggccctgtcatagtggaaatccctcactttgggtccatgagaggaaaagagagagaactcattgttcttcgaagtgaaaatggtgaaacttggaaggagcatcagtttgacagcaaaaatgaagatttaaccgagttacttaatggcatggatgaagaacttgatagcccagaagagttagggaaaaagcgtatctgcaggattatcacgaaagatttcccccagtattttgcagtggtttcccggattaagcaggaaagcaaccagattggtcctgaaggtggaattctgagcagcaccacagtgccccttgttcaagcatctttcccagagggtgccctaactaaaagaattcgagtgggcctccaggcccagcctgttccagatgaaattgtgaaaaagatccttggaaacaaagcaacttttagcccaattgtcactgtggaaccaagaagacggaaattccataaaccaatcacaatgaccattccggtgcccccgccctcaggagaaggtgtatccaatggatacaaaggggacactacacccaatctgcgtcttctctgtagcattacagggggcacttcgcctgctcagtgggaagacatcacaggaacaactcctttgacgtttataaaagattgtgtctcctttacaaccaatgtttcagccagattttggcttgcagactgccatcaagttttagaaactgtggggttagccacgcaactgtacagagaattgatatgtgttccatatatggccaagtttgttgtttttgccaaaatgaatgatcccgtagaatcttccttgcgatgtttctgcatgacagatgacaaagtggacaaaactttagagcaacaagagaattttgaggaagtcgcaagaagcaaagatattgaggttctggaaggaaaacctatttatgttgattgttatggaaatttggccccacttaccaaaggaggacagcaacttgtttttaacttttattctttcaaagaaaatagactgccattttccatcaagattagagacaccagccaagagccctgtggtcgtctgtcttttctgaaagaaccaaagacaacaaaaggactgcctcaaacagcggtttgcaacttaaatatcactctgccagcacataaaaagattgagaaaacagatagacgacagagcttcgcatccttagctttacgtaagcgctacagctacttgactgagcctggaatgagtgagtttcctgacacgtccactaatccgggtcaatgttttaggagaagagacattttttctatgcgctctaaattatgatgtggtttcgaaaataaacgccctgggccaaaaaaaaaaaaaaaaaaaaaaaaaaaa

SEQ ID NO: 48 - DNA, 5865 bp, Homo sapiens
  misc_feature: Incyte ID No 5001859CB1
cgtgcgagcggggccgcgggcgcgcaccgactcaagagccgactgtcagcctcgtgcgggccgtgagttctcctggcgctggtgacaggggcgctgggacaggggcgctgggggcgagccctggcgggggccaggtccgaggaccctgggcgcggcggccccgccaggaggtccggccgcgagcgtgacctcacggggaggggccagcgcggctggactgggcgctgagccgagcgccgggagagcagcgcagaagccgagccgcgaggagcgcactccgtggccccgatggagcggtacaaagccctggaacagctgctgacagagttggatgacttcctcaagattcttgaccaggagaacctgagcagcacagcactggtgaagaagagctgcctggcggagctcctccggctttacaccaaaagcagcagctctgatgaggagtacatttatatgaacaaagtgaccatcaacaagcaacagaatgcagagtctcaaggcaaagcgcctgaggagcagggcctgctacccaatggggagcccagccagcactcctcggcccctcagaagagccttccagacctcccgccacccaagatgattccagaacggaaacagcttgccatcccaaagacggagtctccagagggctactatgaagaggctgagccatatgacacatccctcaatgaggacggagaggctgtgagcagctcctacgagtcctacgatgaagaggacggcagcaagggcaagtcggccccttaccagtggccctcgccggaggccggcatcgagctgatgcgtgacgcccgcatctgcgccttcctgtggcgcaagaagtggctgggacagtgggccaagcagctctgtgtcatcaaggacaacaggcttctgtgctacaaatcctccaaggaccacagccctcagctggacgtgaacctactgggcagcagcgtcattcacaaggagaagcaagtgcggaagaaggagcacaagctgaagatcacaccgatgaatgccgatgtgattgtgctgggcctgcagagcaaggaccaggctgagcagtggctcagggtcatccaggaagtgagcggcctgccttccgaaggagcatctgaaggaaaccagtacaccccggatgcccagcgctttaactgccagaaaccagatatagctgagaagtacctgtcggcttcagagtatgggagctccgtggatggccaccctgaggtcccagaaaccaaagacgtcaagaagaaatgttctgctggcctcaaactgagcaacctaatgaatctgggcaggaagaaatccacctcactggagcctgtggagaggtccctcgagacatccagttacctgaacgtgctggtgaacagccagtggaagtctcgctggtgctctgtcagggacaatcacctgcacttctaccaggaccggaaccggagcaaggtggcccagcaacccctcagcctggtgggctgcgaggtggtcccagaccccagccccgaccacctctactccttccgcatcctccacaagggcgaggagctggccaagcttgaggccaagtcttccgaggaaatgggccactggctgggtctcctgctctctgagtcaggctccaagacagacccagaagagttcacctacgactatgtggatgccgatagggtctcctgtattgtgagtgcggccaaaaactctctcttactgatgcagagaaagttctcagagcccaacacttacatcgatggcctgcctagccaggaccgccaggaggagctgtatgacgacgtggacctgtcagagctcacagctgcggtggagcctaccgaggaagccacccctgttgcagatgacccaaatgagagagaatctgaccgggtgtacctggacctcacacctgtcaagtcctttctgcatggccccagcagtgcacaggcccaggcctcctccccgacgttgtcctgcctggacaatgcaactgaggccctcccggcagactcaggcccaggtcccaccccagatgagccctgcataaagtgtccagagaacctgggagaacagcagctggagagtttggagccagaggatccttccctgagaatcaccaccgtcaaaatccagacggaacagcagagaatctccttcccaccgagctgcccggatgccgtggtggccaccccacctggtgccagcccacctgtgaaggacaggttgcgcgtgaccagtgcagagatcaagcttggcaagaatcggacagaagctgaggtgaagcggtacacagaggagaaggagaggcttgaaaagaagaaggaagaaatccgggggcacctggctcagctccggaaagagaaacgggagctaaaggaaaccctactgaaatgcacagacaaggaagtcctggcgagcctggagcagaagctgaaggaaattgacgaggagtgccggggcgaggagagcaggcgcgtggacctggagctcagcatcatggaggtgaaggacaacctgaagaaggctgaggcagggcctgtgacgttaggcaccaccgtggacaccacccacctggagaatgtgagcccccgccccaaagctgtcacacctgcctctgccccagactgtaccccagtcaactctgcaaccacactcaagaacaggcctctctcggtcgtggtcacaggcaaaggcactgtactccagaaagccaaggaatgggagaagaaaggagcaagttagaaaacaagcttcatctaaagactctcatgtcaatgtggaccttggtgacaatcctgctttgttaaagcaaaaactatgcgaaagggtgagtctgtttagaagaaaaagcaaagactgaggtactgtgaatggagagcttcagctaagaggaggctctgtcccttttcagagccaaaggaaataatacaacaaaaaggaggcttctttggagacctaagtctattggatgtaaacaagacgttgtatttagggatgttctgtgtttctttcttttttgaagttgtcatcaattgctttactaagatttttaaatagtgaaaacctcctgtttagactttggtggaagatgaatcaaggaagcagggccctgtcttatgggtcacatgtctttggtgagtgagaagacctaaactcctggccatcatctcttatccaatacttagcagttggggattaaaccatccttgccttcagttctctccaatattaccaggcccaactcagtcttcagtgattttaaacagcattgacatcatctgtaaaaccatcatctgtaaaaccatctatgacatgagttttgagaaacaataatggggaaaatatttgggaccaagctgaagcactaatcccactaagttaaagacttctttccagtccaaggcaggcctgaatcaactgtctttaaataaaattttaagtgatgctgtattatatataggaaaaaatgcttaaaatcctgtcatttagaacagtgaaaagtatcttttgagattaaagtgactctttactgtaggaaaaatattactctgtgtttacagattcattgctgtggtcaggccatttttaagggaagagttatttaatataaatagtctctgattttaagttctgtttaatgttcattctccttccaagaacaaagtggtgatttttggttagggtgatcgccctcttaaaattggcagtgctgttccttgtgctgcccctgtcttttcctctgatggcatttttttttttttttttttttaacacaggttgaaacatttcatctattatctctgcctcatttctggagggttgtgtatcagttctctaacacttgttcctgagaactaaatgtcttttttattcttatttcctctctcataaacatttggtgaccttttaccaagtggtgagttaggttttttaaaataaaatgttcattgtatttgaagttttcctcagttcatgaaatgacttggggcagggcctggggggttctcaagggaggaaatgttttggttatttacacactgaactacaagatgggatcctagcaggaaactcagcctccttgaatcaaagagcatagataaggctgaaagcaaattttagttgtacagttaacaacataccaaagacacaggaaacatctgcagaaattgatcatatataagtgaatgaaaaatcagtactcctcccagaagttaaatatactcctaaagaattcttgggaaaaaagaaacataccaatactgggaaggttgcctaagtagtaattaaggaagtttatagcataaatgtgtttattttaaaaatgacaaaatcaaaatcgggttcattaaagagacaaatagacaacctaaggcaatatgaaatagcagaactgaaatggaggccttaatacagatactgacaagatttttcaaattataaaaaaatactatgaatatatcagtgtaaaagtttgagaaatatgtattttccattataaggttagtatagctctgataccaaaaattcaaaacaaattgcaaaagaaaacaaatctcattgttggacacagattaaaagaatctgaaataagatgttggcaaatgaaaaattcagcagggtttatagaatgatgcataatgaccaaatagagtttatccaaaaaaatcaagggttgttcaatgattgaaaatacacaaatggataacctatcacattaacagataatacaaaaaatgcattatcttgaggaagaaaaagcataccataaatctcaacatgtaataaacactcttagttcatctaggaattaagcattcttaaactgataagtaacccatagaaaacaattataatgatgaaaaagtcaagaaccagatatggatacatgctacttcatgaaacattattctggaggtcccaggcaatgcaaaatgacaagaaaaagaagtggtatacaactttgaaaggaagagacaaaactagcatcatttggaaatatttctctagaaaagcaagagtcaattattagaactagggtattcaacaagatgactggaatcaggatcgttcagagaaaataaggaaatttatggcctatatgcaatgttttaattattgcaaggaaaatatatttctgtcttatttgaataagtaattaaaggtaaaaattaaagctaatttcttttaaatgaaagaaaaaaggcttggctagttaggccataggatagcactttcctgggggactgacaaaagctgaactatttagtggcactgtcactacaaaaggggaaaaaatgtttcaaaggcgagaaacccatttcctacaaagaaacagtaaaggcccttactatgtaagtcgagagcccacagtttgctgtttaaatccaagtaattcaatcagggcacccaaataccagcccggaacaagacaaaaagcctacattataacatggttaaactgctatttaaagacaatgcattcatgtaatttctatgacatcactgatcagcatctgctagggagcatgtgatcctgccccggacgttgtcttctgtgaagggcagcataataacaaagttatcaatgaaagtatcccttgaatatagatgattatgactcaatgtctggtacaccagactttttaatatctaaaatcttggtgatactgcttcaagcgcttttctagcaccagaaatcctgaagttcttaaagccaaagtacccttgaatgtttgtctataaaacattatgttacaatatttataaagaaaaatgggccaggcacagtggttcacccctgtaatcccagaactttgggaggccaaggcagggggatcacaagcatgagccacagtctctggc

SEQ ID NO: 49 - DNA, 2340 bp, Homo sapiens
  misc_feature: Incyte ID No 7506133CB1
ggcgtggacgcgcgcggggccgccgcgggcacggagtggccgccgcgtcgcctgagcccagagcccgggagtgctctcggccgccgcgtctcctgccctctgtccttccaacccagccctcggctgagccgcgccgcaccatgcccgccgtggacaagctcctgctagaggaggcgttgcaggacagcccccagactcgctctttactgagcgtgtttgaagaagatgctggcaccctcacagactataccaaccagctgctccaggcaatgcagcgcgtctatggagcccagaatgagatgtgcctggccacacaacagctttctaagcaactgctggcatatgaaaaacagaactttgctcttggcaaaggtgatgaagaagtaatttcaacactccactatttttccaaagtggtggatgagcttaatcttctccatacagagctggctaaacagttggcagacacaatggttctacctatcatacaattccgagaaaaggatctcacagaagtaagcactttaaaggatctatttggactcgctagcaatgagcatgacctctcaatggcaaaatacagcaggctgcctaagaaaaaggagaatgagaaggtgaagaccgaagtcggaaaagaggtggccgcggcccggcggaagcagcatctctcctcccttcagtactactgtgccctcaacgcgctgcagtacagaaagcaaatggccatgatggagcccatgataggctttgcccatggacagattaacttttttaagaagggagcagagatgttttccaaacgtatggacagctttttatcctccgttgcagacatggttcaaagcattcaggtagaactggaagccgaggcggaaaagatgcgggtgtcccagcaagaattactttctgttgatgaatctgtttacactccagactctgatgtggccgcaccacagatcaacaggaacctcatccagaaggctggttaccttaatcttagaaacaaaacagggctggtcaccaccacctgggagaggctttatttcttcacccaaggcgggaatctcatgtgtcagcccaggggagccgtggctggaggtttgatccaggacctggacaactgctcagtgatggccgtggattgcgaagaccggcgctactgcttccagatcaccacgcccaatggaaaatcgggaataatcctccaggctgagagcagaaaggaaaatgaagagtggatatgtgcaataaacaacatctccagacagatctacctgaccgacaaccctgaggcagtcgcgatcaagttgaatcagaccgctctgcaagcagtgactcccattacaagttttggaaaaaaacaagaaagctcatgccccagccagaacctgaaaaattcagagatggaaaatgaaaatgacaagattgttcccaaagcaacagccagtctacctgaagcagaggagctgatcgcgcctggaacgccgattcaattcgatattgtgcttcctgctacagaattccttgatcagaacagagggagcaggcgtaccaacccttttggtgaaactgaggatgaatcatttccagaagcagaagattctcttttgcagcagatgtttatagttcggtttttgggatcaatggcagttaaaacagacagcactactgaagtgatttatgaagcgatgagacaagtattggctgctcgggctattcataacatcttccgcatgacagaatcccatctgatggtcaccagtcaatctttgaggttgatagatccacagactcaagtatcaagggccaatatatgttatgctattaatttgggaaaagaaattattgaggttcagaaggatccagaagcactggctcaattaatgctgtccataccactaaccaatgatggaaaatatgtactgttaaacgatcaaccagatgacgatgatggaaatccaaatgaacatagaggcgcagaatccgaagcataactcacttgcgcctgtgggggaagagcaaacaggaaggagagctacctcctaagggttttaacgtctctgacatacaggcacactgacctgatttccgaaggctgacaatcgtttgtggaatgtaatcttgatgccttgatactgagacttgggagggaaactaagaaatggttgacagcgttcccacccatctacaatgttattttaggtgctttgtggtaagtcttttttcttagattgcgctaaaatttcttagattgttcagcgctcagaacaaaagtttgaaaaatgcattgttcatatgaatgtcatctcttttcagtttccagtatcctttttaaagaat

SEQ ID NO: 50 - DNA, 3263 bp, Homo sapiens
  misc_feature: Incyte ID No 5301066CB1
gagtgaaattcttggaccggcgcaagacggcggcccccgagccgccgccgctgtccggagccccacaggacggcatcagaattaatgtaactacactgaaagatgatggggactgccgccgccgcggtggcactgcctgatgctcggcccagtgtgccggtgccccgctgggtgacagtggactcccagggcgcagcaggagcaggtgacagactgttggctgaaggtgagggtgtccaccctcgcaggcacacgttccagggagcacggcactcaagcagggccgtcagactgggctctggtgcccagaagctgtgcaggccggagagaagcactaccacccttcctgcgcgctatgtgtcaggtgcggccagatgtttgcagaaggcgaagagatgtatcttcaaggttcctccatctggcatccggcgtgtcgacaagcagccagaactgaagacagaaacaaggaaaccagaacttcctcagagagcatcatttctgtccctgcttccagcacctcagggtctccgagccgtgtgatttatgccaagcttggtggtgagatcctggactacagggacttggcagcccttcctaaaagtaaggccatctatgacatcgaccgccccgacatgatctcctactcaccctacatcagccactctgcaggggacaggcagagctacggcgagggggatcaggatgaccggtcctacaagcagtgtcggacctccagcccaagctccactgggtcggttagcctcgggcgctacactccgacctcacggtcaccacagcactacagccgtccagctggtactgtgagtgtgggtaccagtagctgcctctccctgtcccaacacccaagccctacatccgtgttcagacatcattacatcccctacttccgaggcagtgaaagtggccggagcacccccagcctctccgtgctctctgacagcaagccgcccccctccacctaccagcaggcacctcgccacttccacgtcccagacactggcgtaaaagataacatctataggaaaccccctatctacagacagcatgctgccaggcgatcggatggggaggatggaagcttggaccaggataacaggaagcagaagagcagctggctgatgctcaatggggatgcagacaccaggaccaattctccagacctggacacccagtccttgtcccacagcagcgggaccgacagagaccctctccaaaggatggcagggacagctgtcactcacgattcccctatttccaaatctgaccctctcccaggacatggaaagaatggcttggaccagcggaatgccaatctggccccctgtggagcagacccggatgccagctggggcatgcgagaatacaagatctatccgtatgactccctcatcgtcacaaaccgaattcgcgtgaaactgcccaaagacgtggaccggacgagactggagagacacttgtcgcccgaggagttccaggaagtgtttgggatgagcatcgaggagtttgaccgcctggccctctggaagaggaatgaccttaagaagaaagcccttttgttctgacggctgccagcctgccccactggtgtgtgccgggcgccgaggccaggggcccctggcgagaaccgcacacacccctcccacacaccttgctctggcttctctgtgtccatggggtgggcgggagggggtcccccagcaggtgcggcccctgcacctgccggcgacactcctgccggtagtttagggccgagacggctagcttcacgccacccttccccgctgtggcttggtgtcagggagaggctgtagagtggctgtgtcgggcatcagatggagcacacaggtggctgcagcccagcccaccttcccagcgttctcgaggtgccctggccccggtgctgggcacgtgggggacagaggtggccgggacgtgagctgtgaggcttgttgatgacgggtgctgacaccatcatcgggggtgggcacacggcccttcggagcctgggcagcctggcctcacaggcagactcgcagacggggcagtgagcgtctgggacagtgccaagagtggggtgtgtgatttttgcaggcgtctgtgatgggtctctttagggacaaatgtcaacaagggacaagacagggcaccttccgccagcgcccctccatgcgctgcgttcctcctccagatcgacccctagatgcctacacaatatctttaaagtaacacagaagtttttattttattaaaaagtatagcctacttaaacgcgagatgacatatatagagtttaattttacggtccctccgcaggggagcggcctccagccttattctccaccgctcggatctgtgtggtttcagtctgttcttggtgtggtcctcatacacagagctccctgtctagtttcttttctttttcttttttctttttctcgtcacacaggtaaccttaaagacagacccctctaaagaacgctttgtaaatacatgtgaggtatagccaccactgttttccttgctgttatttttccaagtcttggggagaaaacatcctcctctgatggccaaagccctggaatcaagggtttccacgtaccctgcctaatacccgacgtagctcttgatgcaccgtccttgtgctgtggctggcggtgtctcagcctgaaacataaaccccacatgccccaggagcgatgtgctccctgaaacagacaaccacacgctgttggggagagaaggatggagataggatggagataggatggagatgtggctcttctcatctttgaagccagcagggcatcccggagcaggaggctggccgggctccccaagcgaaggcgttggtgtctgtcattaggtgtgtgttagggtgcagcaccggccgtcacaggatgctgataagcgcgctgagaggtggatgaaacaccaaagtctgtttccccgtccgcagtgggtgttgcctctttgtgtgtgtcccgatgttcctgcctgtgagtcggccttactccgtttccttagcgcccatgacacgccaagtcccgtttcgcactcggcttctcacccgcccagctcggctagggagggggagtttttagcacctaatatgcttcctgccattgcgcaatctgagcctgagcaactggaaacccccatttctcattagtgcaatgtcatatctgatcccaggaagcctggaaaataaaagacgatgcattataaaaaaaaaaaaaaaaaaa

(19) United States
(12) Patent Application Publication
(10) Pub. No.: US 2005/0027103 A1
(43) Pub. Date: Feb. 3, 2005

(54) Structural and cytoskeleton-associated protein

(76) Inventors: Y. Tom Tang, San Jose, CA (US); Bridget A. Warren, San Marcos, CA (US); Cynthia D. Honchell, San Carlos, CA (US); Thomas W. Richardson, Redwood City, CA (US); Vicki S. Elliott, San Jose, CA (US); Narinder K. Chawla, Union City, CA (US); Henry Yu, Sunnyvale, CA (US); Sajeev Batra, Oakland, CA (US); Jennifer A. Griffin, Fremont, CA (US); Mariah R. Baughn, Los Angeles, CA (US); Ian J. Forsythe, Edmonton (CA); Neil Burford, Durham, CT (US); Brooke M. Emerling, Chicago, IL (US); Madhusudan M. Sanjanwala, Los Altos, CA (US); Farrah A. Khan, Des Plaines, CA (US); Dyung Aina M. Lu, San Jose, CA (US); April J A Hafalia, Daly City, CA (US); Danniel B. Nguyen, San Jose, CA (US); Junming Yang, San Jose, CA (US); Joana L. Xi, Millbrae, CA (US); Shanya D. Becha, San Francisco, CA (US); Monique G. Yao, Mountain View, CA (US); Kimberly J. Gietzen, San Jose, CA (US); Wen Luo, San Diego, CA (US); Ernestine A. Lee, Kensington, CA (US); Craig H. Ison, San Jose, CA (US); Amy W. Lasek, Oakland, CA (US)

Correspondence Address: FOLEY AND LARDNER;SUITE 500, 3000 K STREET NW, WASHINGTON, DC, 20007, US

(21) Appl. No.: 10/479,764

(22) PCT Filed: Jun. 6, 2002
(86) PCT No.: PCT/US02/17956

Related U.S. Application Data
(60) Provisional application No. 60/296,865, filed on Jun. 7, 2001.
(60) Provisional application No. 60/296,878, filed on Jun. 8, 2001.
(60) Provisional application No. 60/298,664, filed on Jun. 15, 2001.
(60) Provisional application No. 60/300,149, filed on Jun. 21, 2001.
(60) Provisional application No. 60/302,340, filed on Jun. 29, 2001.
(60) Provisional application No. 60/303,481.
(60) Provisional application No. 60/305,059, filed on Jul. 12, 2001.
(60) Provisional application No. 60/343,557, filed on Dec. 21, 2001.

(51) Int. Cl. C12Q 1/68; C07H 21/04; C07K 14/47

(52) U.S. Cl. 530/350; 435/6; 435/69.1; 435/320.1; 435/325; 536/23.5

(57) ABSTRACT

Various embodiments of the invention provide human structural and cytoskeleton-associated proteins (SCAP) and polynucleotides which identify and encode SCAP. Embodiments of te invention also provide expression vectors, host cells, anti-bodies, agonists, and antagonists. Other embodiments provide methods for diagnosing, treating, or preventing discorders associated with aberrant expression of SCAP.

### TECHNICAL FIELD

The invention relates to novel nucleic acids, structural and cytoskeleton-associated proteins encoded by these nucleic acids, and to the use of these nucleic acids and proteins in the diagnosis, treatment, and prevention of cell proliferative disorders, viral infections, and neurological disorders. The invention also relates to the assessment of the effects of exogenous compounds on the expression of nucleic acids and structural and cytoskeleton-associated proteins.

### BACKGROUND OF THE INVENTION

The cytoskeleton is a cytoplasmic network of protein fibers that mediate cell shape, structure, and movement. The cytoskeleton supports the cell membrane and forms tracks along which organelles and other elements move in the cytosol. The cytoskeleton is a dynamic structure, that allows cells to adopt various shapes and to carry out directed movements. Major cytoskeletal fibers include the microtubules, the microflaments, and the intermediate filaments. Motor proteins, including myosin, dynein, and kinesin, drive movement of or along the fibers. The motor protein dynamin drives the formation of membrane vesicles. Accessory or associated proteins modify the structure or activity of the fibers while cytoskeletal membrane anchors connect the fibers to the cell membrane.

Microtubules and Associated Proteins

Tubulins

Microtubules, cytoskeletal fibers with a diameter of about 24 nm, have multiple roles in the cell. Bundles of microtubules form cilia and flagella, which are whip-like extensions of the cell membrane that are necessary for sweeping materials across an epithelium and for swimming of sperm, respectively. Marginal bands of microtubules in red blood cells and platelets are important for these cells' pliability. Organelles, membrane vesicles, and proteins are transported in the cell along tracks of microtubules. For example, microtubules run through nerve cell axons, allowing bi-directional transport of materials and membrane vesicles between the cell body and the nerve terminal. Failure to supply the nerve terminal with these vesicles blocks the transmission of neural signals. Microtubules are also critical to chromosomal movement during cell division. Both stable and short-lived populations of microtubules exist in the cell

Microtubules are polymers of GTP-binding tubulin protein subunits. Bach subunit is a heterodimer of α- and β-tubulin, multiple isoforms of which exist The hydrolysis of GTP is linked to the addition of tubulin subunits at the end of a microtubule. The subunits interact head to tail to form protofilaments; the protofilaments interact side to side to form a microtubule. A microtubule is polarized, one end ringed with α-tubulin and the other with β-tubulin, and the two ends differ in their rates of assembly. Generally, each microtubule is composed of 13 protofilaments although 11 or 15 protofilament-microtubules are sometimes found. Cilia and flagella contain doublet microtubules. Microtubules grow from specialized structures known as centrosomes or microtubule-organizing centers (MTOCs). MTOCs may contain one or two centrioles, which are pinwheel arrays of triplet microtubules. The basal body, the organizing center located at the base of a cilium or flagellum, contains one centriole. Gamma tubulin present in the MTOC is important for nucleating the polymerization of α- and β-tubulin heterodimers but does not polymerize into microtubules. The protein pericentrin is found in the MTOC and has a role in microtubule assembly.

Microtubule-Associated Proteins

Microtubule-associated proteins (MAPs) have roles in the assembly and stabilization of microtubules. One major family of MAPs, assembly MAPs, can be identified in neurons as well as non-neuronal cells. Assembly MAPs are responsible for cross-linking microtubules in the cytosol. These MAPs are organized into two domains: a basic microtubule-binding domain and an acidic projection domain. The projection domain is the binding site for-membranes, intermediate-filaments, or other microtubules. Based on sequence analysis, assembly MAPs can be further grouped into two types: Type I and Type II Type I MAPs, which include MAP1A and MAP1B, are large, filamentous molecules that co-purify with microtubules and are abundantly expressed in brain and testes. Type I MAPs contain several repeats of a positively-charged amino acid sequence motif that binds and neutralizes negatively charged tubulin, leading to stabilization of microtubules. MAP1A and MAP1B are each derived from a single precursor polypeptide that is subsequently proteolytically processed to generate one heavy chain and one light chain.

Another light chain, LC3, is a 16.4 kDa molecule that binds MAP1A, MAP1B, and microtubules. It is suggested that LC3 is synthesized from a source other than the MAP1A or MAP1B transcripts, and that the expression of LC3 maybe important in regulating the microtubule binding activity of MAP1A and MAP1B during cell proliferation (Mann, S. S. et al. (1994) J. Biol. Chem. 269:11492-11497).

Type II MAPs, which include MAP2a, MAP2b, MAP2c, MAP4, and Tau, are characterized by three to four copies of an 18-residue sequence in the microtubule-binding domain. MAP2a, MAP2b, and MAP2c are found only in dendrites, MAP4 is found in non-neuronal cells, and Tau is found in axons and dendrites of nerve cells. Alternative splicing of the Tau mRNA leads to the existence of multiple forms of Tau protein. Tau phosphorylation is altered in neurodegenerative disorders such as Alzheimer's disease, Pick's disease, progressive supranuclear palsy, corticobasal degeneration, and familial frontotemporal dementia and Parkinsonism linked to chromosome 17. The altered Tau phosphorylation leads to a collapse of the microtubule network and the formation of intraneuronal Tau aggregates (Spillantini, M. G. and M. Goedert (1998) Trends Neurosci. 21:428-433).

Another microtubule associated protein, STOP (stable tubule only polypeptide), is a calmodulin-regulated protein that regulates stability (Denarier, E. et al. (1998) Biochem. Biophys. Res. Commun. 24:791-796). In order for neurons to maintain conductive connections over great distances, they rely upon axodendritic extensions, which in turn are supported by microtubules. STOP proteins function to stabilize the microtubular network. STOP proteins are associated with axonal microtubules, and are also abundant in neurons (Guillaud, L. et at (1998) J. Cell Biol. 142:167-179). STOP proteins are necessary for normal neurite formation, and have been observed to stabilize microtubules, in vitro, against cold-, calcium-, or drug-induced dissassembly (Margolis, R. L. et al. (1990) EMBO 9:4095-502).

Microfilaments and Associated Proteins

Actins

Microfilaments, cytoskeletal filaments with a diameter,of about 7-9 nm, are vital to cell locomotion, cell shape, cell adhesion, cell division, and muscle contraction. Assembly and disassembly of the microfilaments allow cells to change their morphology. Microfilaments are the polymerized form of actin, the most abundant intracellular protein in the eukaryotic cell. Human cells contain six isoforms of actin. The three α-actins are found in different kinds of muscle, nonmuscle β-actin and nonmuscle γ-actin are found in nonmuscle cells, and another γ-actin is found in intestinal smooth muscle cells. G-actin, the monomeric form of actin, polymerizes into polarized, helical F-actin filaments, accompanied by the hydrolysis of ATP to ADP. Actin filaments associate to form bundles and networks, providing a framework to support the plasma membrane and determine cell shape. These bundles and networks are connected to the cell membrane. In muscle cells, thin filaments containing actin slide past thick filaments containing the motor protein myosin during contraction. A family of actin-related proteins exist that are not part of the actin cytoskeleton, but rather associate with microtubules and dynein.

Actin-Associated Proteins

Actin-associated proteins have roles in cross-linking, severing, and stabilization of actin filaments and in sequestering actin monomers. Several of the actin-associated proteins have multiple functions. Bundles and networks of actin filaments are held together by actin cross-linking proteins. These proteins have two actin-binding sites, one for each filament. Short cross-liking proteins promote bundle formation while longer, more flexible cross-linking proteins promote network formation. Actin-interacting proteins (AIPs) participate in the regulation of actin filament organization. Other actin-associated proteins such as TARA, a novel F-actin binding protein, function in a similar capacity by regulating actin cytoskeletal organization. Calmodulin-like calcium-binding domains in actin cross-linking proteins allow calcium regulation of cross-linking. Group I cross-linking proteins have unique actin-binding domains and include the 30 kD protein, EF-1a, fascin, and scruin. Group II cross-linking proteins have a 7,000-MW actin-binding domain and include villin and dematin. Group III cross-linking proteins have pairs of a 26,000-MW actin-binding domain and include fimbrin, spectrin, dystrophin, ABP 120, and filamin.

Severing proteins regulate the length of actin filaments by breaking them into short pieces or by blocking their ends. Severing proteins include gCAP39, severin (fragmin), gelsolin, and villin. Capping proteins can cap the ends of actin filaments, but cannot break filaments. Capping proteins include CapZ and tropomodulin. The proteins thymosin and profilin sequester actin monomers in the cytosol, allowing a pool of unpolymerized actin to exist. The actin-associated proteins tropomyosin, troponin, and caldesmon regulate muscle contraction in response to calcium.

Microtubule and actin filament networks cooperate in-processes such as vesicle and organelle transport, cleavage furrow placement, directed cell migration, spindle rotation, and nuclear migration. Microtubules and actin may coordinate to transport vesicles, organelles, and cell fate determinants, or transport may involve targeting and capture of microtubule ends at cortical actin sites. These cytoskeletal systems may be bridged by myosin-kinesin complexes, myosin-CLIP170 complexes, formin-homology (FH) proteins, dynein, the dynactin complex, Kar9p, coronin, ERM proteins, and kelch repeat-containing proteins (for a review, see Goode, B. L. et al. (2000) Curr. Opin. Cell Biol. 12:63-71). The kelch repeat is a motif originally observed in the kelch protein, which is involved in formation of cytoplasmic bridges called ring canals. A variety of mammalian and other kelch family proteins have been identified. The kelch repeat domain is believed to mediate interaction with actin (Robinson, D. N. and L. Cooley (1997) J. Cell Biol. 138:799-810).

ADF/cofilins are a family of conserved 15-18 kDa actin-binding proteins that play a role in cytokinesis, endocytosis, and in development of embryonic tissues, as well as in tissue regeneration and in pathologies such as ischemia, oxidative or osmotic stress. LIM kinase 1 downregulates ADF (Carlier, M. F. et al. (1999) J. Biol. Chem. 274:33827-33830).

LIM is an acronym of three transcription factors, Lin-11, Isl-1, and Mec-3, in which the motif was first identified. The LIM domain is a double zinc-finger motif that mediates the protein-protein interactions of transcription factors, signaling, and cytoskeleton-associated proteins (Roof, D. J. et al. (1997) J. Cell Biol. 138:575-588). These proteins are distributed in the nucleus, cytoplasm, or both (Brown, S. et al. (1999) J. Biol. Chem. 274:27083-27091). Recently, ALP (actinin-associated LIM protein) has been shown to bind alpha-actinin-2 (Bouju, S. et al. (1999) Neuromuscul. Disord. 9:3-10).

The Frabin protein is another example of an actin-filament binding protein (Obaishi, H. et al. (1998) J. Biol. Chem. 273:18697-18700). Frabin (FGD1-related F-actin-binding protein) possesses one actin-filament binding (FAB) domain, one Dbl homology (DH) domain, two pleckstrin homology (PH) domains, and a single cysteine-rich FYVE (Fab1p, XOTB, Yac1p, and EEA1 (early endosomal antigen 1)) domain. Frabin has shown GDP/GTP exchange activity for Cdc42 small G protein (Cdc42), and indirectly induces activation of Rac small G protein (Rac) in intact cells. Through the activation of Cdc42 and Rac, Frabin is able to induce formation of both filopodia- and lamellipodia-like processes (Ono, Y. et al. (2000) Oncogene 19:3050-3058).

The Rho family of small GTP-binding proteins are important regulators of actin-dependent cell functions including cell shape change, adhesion, and motility. The Rho family consists of three major subfamilies: Cdc42, Rac, and Rho. Rho family members cycle between GDP-bound inactive and GTP-bound active forms by means of a GDP/GTP exchange factor (GEF) (Umikawa, M. et al. (1999) J. Biol. Chem. 274:25197-25200). The Rho GEF family is crucial for microfilament organization.

Intermediate Filaments and Associated Proteins

Intermediate filaments (IFs) are cytoskeletal fibers with a diameter of about 10 nm, intermediate between that of microfilaments and microtubules. IFs serve structural roles in the cell, reinforcing cells and organizing cells into tissues. IFs are particularly abundant in epidermal cells and in neurons. IFs are extremely stable, and, in contrast to microfilaments and microtubules, do not function in cell motility.

Five types of IF proteins are known in mammals. Type I and Type II proteins are the acidic and basic keratins, respectively. Heterodimers of the acidic and basic keratins are the building blocks of keratin IFs. Keratins are abundant in soft epithelia such as skin and cornea, hard epithelia such as nails and hair, and in epithelia that line internal body cavities. Mutations in keratin genes lead to epithelial diseases including epidermolysis bullosa simplex, bullous congenital ichthyosiform erythroderma (epidermolytic hyperkeratosis), non-epidermolytic and epidermolytic palmoplantar keratoderma, ichthyosis bullosa of Siemens, pachyonychia congenita, and white sponge nevus. Some of these diseases result in severe skin blistering. (See, e.g., Wawersik, M. et al. (1997) J. Biol. Chem. 272:32557-32565; and Corden L. D. and W. H. McLean (1996) Exp. Dermatol. 5:297-307.)

Type III IF proteins include desmin, glial fibrillary acidic protein, vimentin, and peripherin. Desmin filaments in muscle cells link myofibrils into bundles and stabilize sarcomeres in contracting muscle. Glial fibrillary acidic protein filaments are found in the glial cells that surround neurons and astrocytes. Vimentin filaments are found in blood vessel endothelial cells, some epithelial cells, and mesenchymal cells such as fibroblasts, and are commonly associated with microtubules. Vimentin filaments may have roles in keeping the nucleus and other organelles in place in the cell. Type IV IFs include the neurofilaments and nestin. Neurofilaments, composed of three polypeptides, NF-L, NF-M, and NF-H, are frequently associated with microtubules in axons. Neurofilaments are responsible for the radial growth and diameter of an axon, and ultimately for the speed of nerve impulse transmission. Changes in phosphorylation and metabolism of neurofilaments are observed in neurodegenerative diseases including amyotrophic lateral sclerosis, Parkinson's disease, and Alzheimer's disease (Julien, J. P. and W. E. Mushynski (1998) Prog. Nucleic Acid Res. Mol. Biol. 61:1-23). Type V IFs, the lamins, are found in the nucleus where they support the nuclear membrane.

IFs have a central α-helical rod region interrupted by short nonhelical linker segments. The rod region is bracketed, in most cases, by non-helical head and tail domains. The rod regions of intermediate filament proteins associate to form a coiled-coil dimer. A highly ordered assembly process leads from the dimers to the IFs. Neither ATP nor GTP is needed for IP assembly, unlike that of microfilaments and microtubules.

IF associated proteins (IFAPs) mediate the interactions of IFs with one another and with other cell structures. IFAPs cross-link IFs into a bundle, into a network, or to the plasma membrane, and may cross-link IFs to the microfilament and microtubule cytoskeleton. Microtubules and IFs are, particularly closely associated. IFAPs include BPAG1, plakoglobin, desmoplakin I, desmoplakin II, plectin, filaggrin, and lamin B receptor.

Cytoskeletal-Membrane Anchors

Cytoskeletal fibers are attached to the plasma membrane by specific proteins. These attachments are important for maintaining cell shape and for muscle contraction. In erytbrocytes, the spectrin-actin cytoskeleton is attached to the cell membrane by three proteins, band 4.1, ankyrin, and adducin. Defects in this attachment result in abnormally shaped cells which are more rapidly degraded by the spleen, leading to anemia. In platelets, the spectrin-actin cytoskeleton is also linked to the membrane by a ankyrin; a second actin network is anchored to the membrane by filamin. In muscle cells the protein dystrophin links actin filaments to the plasma membrane; mutations in the dystrophin gene lead to Duchenne muscular dystrophy.

Focal Adhesions

Focal adhesions are specialized structures in the plasma membrane involved in the adhesion of a cell to a substrate, such as the extracellular matrix (ECM). Focal adhesions form the connection between an extracellular substrate and the cytoskeleton, and affect such functions as cell shape, cell motility and cell proliferation. Transmembrane integrin molecules form the basis of focal adhesions. Upon ligand binding, integrins cluster in the plane of the plasma membrane. Cytoskeletal linker proteins such as the actin binding proteins α-actinin, talin, tensin, vinculin, paxillin, and filamin are recruited to the clustering site. Key regulatory proteins, such as Rho and Ras family proteins, focal adhesion kinase, and Src family members are also recruited. These events lead to the reorganization of actin filaments and the formation of stress fibers. These intracellular rearrangements promote further integrin-ECM interactions and integrin clustering. Thus, integrins mediate aggregation of protein complexes on both the cytosolic and extracellular faces of the plasma membrane, leading to the assembly of the focal adhesion. Many signal transduction responses are mediated via various adhesion complex proteins, including Src, FAY, paxillin, and tensin. For a review, see Yamada, K. M. and B. Geiger, (1997) Curr. Opin. Cell Biol. 9.76-85.)

IFs are also attached to membranes by cytoskeletal-membrane anchors. The nuclear lamina is attached to the inner surface of the nuclear membrane by the lamin B receptor. Vimentin IFs are attached to the plasma membrane by ankyrin and plectin. Desmosome and hemidesmosome membrane junctions hold together epithelial cells of organs and skin. These membrane junctions allow shear forces to be distributed across the entire epithelial cell layer, thus providing strength and rigidity to the epithelium. IFs in epithelial cells are attached to the desmosome by plakoglobin and desmoplakins. The proteins that link IFs to hemidesmosomes are not known. Desmin IFs surround the sarcomere in muscle and are linked to the plasma membrane by paranemin, synemin, and ankyrin.

Motor Proteins

Myosin-Related Motor Proteins

Myosins are actin-activated ATPases; found in eukaryotic cells, that couple hydrolysis of ATP with motion. Myosin provides the motor function for muscle contraction and intracellular movements such as phagocytosis and rearrangement of cell contents during mitotic cell division (cytokinesis). The contractile unit of skeletal muscle, termed the sarcomere, consists of highly ordered arrays of thin actin-containing filaments and thick myosin-containing filaments. Crossbridges form between the thick and thin filaments, and the ATP-dependent movement of myosin heads within the thick filaments pulls the thin filaments, shortening the sarcomere and thus the muscle fiber.

Myosins are composed of one or two heavy chains and associated light chains. Myosin heavy chains contain an amino-terminal motor or head domain, a neck that is the site of light-chain binding, and a carboxy-terminal tail domain. The tail domains may associate to form an α-helical coiled coil. Conventional myosins, such as those found in muscle tissue, are composed of two myosin heavy-chain subunits, each associated with two light-chain subunits that bind at the neck region and play a regulatory role. Unconventional myosins, believed to function in intracellular motion, may contain either one or two heavy chains and associated light chains. There is evidence for about 25 myosin heavy chain genes in vertebrates, more than half of them unconventional.

Dynein-Related Motor Proteins

Dyneins are (−) end-directed motor proteins which act on microtubules. Two classes of dyneins, cytosolic and axonemal, have been identified. Cytosolic dyneins are responsible for translocation of materials along cytoplasmic microtubules, for example, transport from the nerve terminal to the cell body and transport of endocytic vesicles to lysosomes. As well, viruses often take advantage of cytoplasmic dyneins to be transported to the nucleus and establish a successful infection (Sodeik, B. et al. (1997) J. Cell Biol. 136:1007-1021). Virion proteins of herpes simplex virus 1, for example, interact with the cytoplasmic dynein intermediate chain (Ye, G. J. et al. (2000) J. Virol. 74:1355-1363). Cytoplasmic dyneins are also reported to play a role in mitosis. Axonemal dyneins are responsible for the beating of flagella and cilia. Dynein on one microtubule doublet walks along the adjacent microtubule doublet. This sliding force produces bending that causes the flagellum or cilium to beat. Dyneins have a native mass between 1000 and 2000 kDa and contain either two or three force-producing heads driven by the hydrolysis of ATP. The heads are linked via stalks to a basal domain which is composed of a highly variable number of accessory intermediate and light chains. Cytoplasmic dynein is the largest and most complex of the motor proteins.

Nebulin-Related Proteins

Nebulin is a large sarcomeric protein that interacts with actin filaments in skeletal muscle (Wang, K. et al. (1996) J. Biol. Chem. 271:4304-4314). Nebulin contains 185 or more copies of a 35-residue module that has a consensus sequence and a predicted α-helical structure. The 35-residue module comprises an actin-binding domain. In the central region of nebulin, the 35-residue modules exhibit a seven module super-repeat pattern. This super-repeat pattern is not present in the C-terminal 100 kDa region of nebulin. The N-terminal region of nebulin contains 8 linker modules and an 8 kDa acidic domain. The C-terminal region is distinct and contains an SH3 domain. Within the sarcomere, nebulin is oriented with its C-terminus located at the Z-line, and its N-terminus at the pointed slow-growing end of thin filaments in the acto-myosin overlap region.

Nebulin exists as different isoforms which range in size from 600-900 kDa (Kruger, M. et al. (1991) J. Cell. Biol. 115:97-107). The size of nebulin is tissue- and species-specific and is developmentally regulated. Based on the observation that isoform size correlates with the length of thin filaments in skeletal muscle, nebulin is proposed to play a role as a molecular ruler that regulates the length of thin filaments. Each nebulin 35-residue module may associate with one actin monomer; thus, isoforms with different numbers of modules could determine the length of thin filaments. The N-terminal region of nebulin interacts with tropomodulin, which may assist in this function (McElhinny, A. S. et al. (2001) J. Biol. Chem. 276:583-592). Tropomodulin caps actin at the pointed end of thin filaments and maintains filament length by preventing actin monomer dissociation or addition.

Nebulin is absent from cardiac muscle, but related proteins with nebulin-like modules may provide similar functions. Nebulette, for example, is specifically expressed in heart and has a C-terminal region containing twenty-three 35-residue nebulin-like modules (Moncman, C. L. and Wang, K. (2000) J Muscle Res. Cell Motil. 21:153-169; Millevoi, S. et al. (1998) J. Mol. Biol. 282:111-123). The domain structure of nebulette is similar to nebulin, though it is a smaller protein of only 107 kDa. It has an acidic N-terminal domain, a repeat domain containing nebulin-like modules, a linker domain, and an SH3 domain. The repeat domain of nebulette is about one-tenth the size of that of nebulin. The 35-residue modules of nebulette have a consensus motif, and a subfamily of modules 15-22 share a conserved motif. Unlike nebulin, nebulette modules do not display a super-repeat pattern. Nebulette binds to actin as well as other sarcomeric proteins including myosin, calmodulin, tropomyosin, troponin, and α-actinin (Moncman, C. L. and Wang, K. (1999) Cell Motil. Cytoskeleton 44:1-22). The orientation of nebulette in the sarcomere is analogous to that of nebulin with its C-terminus at the Z-line and its N-terminus in the I-band.

Nebulin-related anchoring protein (N-RAP) is expressed in cardiac and skeletal muscle (Luo, G. et al. (1997) Cell Motil. Cytoskeleton 38:75-90). It is a 133 kDa protein found at the ends of myofibrils at muscle myotendon junctions and intercalated disks. The C-terminal region of N-RAP has 27 copies of the 35-residue nebulin-like modules. Seventeen of the modules are organized in a super-repeat pattern. The N-terminal region contains a cysteine-rich LIM domain. LIM domains bind two zinc ions in two adjacent zinc finger-like structures and are known to mediate protein-protein interactions. N-RAP may mediate interactions between actin filaments of myofibrils and other sarcomeric proteins. N-RAP binds to actin, talin, and vinculin (Luo, G. et al. (1999) Biochemistry 38:6135-6143). It interacts with actin and vinculin through its super-repeat region and with talin through its LIM domain. Talin and vinculin are also located at myotendon junctions and together with N-RAP may provide a link between actin filaments of the myofibril and the sarcolemma and transmit tension from the myofibril to the extracellular matrix

Mutations of sarcomeric proteins are associated with muscle weakness and disease (Laing, N. G. (1999) Curr. Opin. Neurol. 12:513-518). Autosomal recessive nemaline myopathy in some cases is caused by nebulin deficiency (Pelin, K. et al. (1999) Proc. Natl. Acad. Sci. 96:2305-2310). The disease is characterized by the presence of nemaline bodies in muscle fibers. The nemaline bodies contain proteins normally associated with the Z disc and thin filament. Defects in nebulin apparently perturb interactions among sarcomeric proteins and result in the pathological aggregation of proteins in nemaline bodies.

Kinesin-Related Motor Proteins

Kinesins are (+) end-directed motor proteins which act on microtubules. The prototypical kinesin molecule is involved in the transport of membrane-bound vesicles and organelles. This function is particularly important for axonal transport in neurons. Kinesin is also important in all cell types for the transport of vesicles from the Golgi complex to the endoplasmic reticulum. This role is critical for maintaining the identity and functionality of these secretory organelles.

Kinesins define a ubiquitous, conserved family of over 50 proteins that can be classified into at least 8 subfamilies based on primary amino acid sequence, domain structure, velocity of movement, and cellular function. (Reviewed in Moore, J. D. and S. A. Endow (1996) Bioessays 18:207-219; and Hoyt, A. M. (1994) Curr. Opin. Cell Biol. 6:63-68.) The prototypical kinesin molecule is a heterotetramer comprised of two heavy polypeptide chains (KHCs) and two light polypeptide chains (KLCs). The KHC subunits are typically referred to as “kinesin.” KHC is about 1000 amino acids in length, and KLC is about 550 amino acids in length. Two KHCs dimerize to form a rod-shaped molecule with three distinct regions of secondary structure. At one end of the molecule is a globular motor domain that functions in ATP hydrolysis and microtubule binding. Kinesin motor domains are highly conserved and share over 70% identity. Beyond the motor domain is an α-helical coiled-coil region which mediates dimerization. At the other end of the molecule is a fan-shaped tail that associates with molecular cargo. The tail is formed by the interaction of the KHC C-termini with the two KLCs.

Members of the more divergent subfamilies of kinesins are called kinesin-related proteins (KRPs), many of which function during mitosis in eukaryotes (Hoyt, supra). Some KRPs are required for assembly of the mitotic spindle. In vivo and in vitro analyses suggest that these KRPs exert force on microtubules that comprise the mitotic spindle, resulting in the separation of spindle poles. Phosphorylation of KRP is required for this activity. Failure to assemble the mitotic spindle results in abortive mitosis and chromosomal aneuploidy, the latter condition being characteristic of cancer cells. In addition, a unique KRP, centromere protein E, localizes to the kinetochore of human mitotic chromosomes and may play a role in their segregation to opposite spindle poles.

Dynamin-Related Motor Proteins

Dynamin is a large GTPase motor protein that functions as a “molecular pinchase,” generating a mechanochemical force used to sever membranes. This activity is important in forming clathrin-coated vesicles from coated pits in endocytosis and in the biogenesis of synaptic vesicles in neurons. Binding of dynamin to a membrane leads to dynamin's self-assembly into spirals that may act to constrict a flat membrane surface into a tubule. GTP hydrolysis induces a change in conformation of the dynamin polymer that pinches the membrane tubule, leading to severing of the membrane tubule and formation of a membrane vesicle. Release of GDP and inorganic phosphate leads to dynamin disassembly. Following disassembly the dynamin may either dissociate from the membrane or remain associated to the vesicle and be transported to another region of the cell. Three homologous dynamin genes have been discovered, in addition to several dynamin-related proteins. Conserved dynamin regions are the N-terminal GTP-binding domain, a central pleckstrin homology domain that binds membranes, a central coiled-coil region that may activate dynamin's GTPase activity, and a C-terminal proline-rich domain that contains several motifs that bind SH3 domains on other proteins. Some dynamin-related proteins do not contain the pleckstrin homology domain or the proline-rich domain (See McNiven, M. A. (1998) Cell 94:151-154; Scaife, R. M. and R. L. Margolis (1997) Cell. Signal. 9:395401.)

The cytoskeleton is reviewed in Lodish, H. et al. (1995) Molecular Cell Biology, Scientific American Books, New York N.Y.

Cyclic Nucleotide Signaling

Cyclic nucleotides (cAMP and cGMP) function as intracellular second messengers to transduce a variety of extracellular signals including hormones, light, and neurotransmitters. In particular, cyclic-AMP dependent protein kinases (PKA) are thought to account for all of the effects of cAMP in most mammalian cells, including various hormone-induced cellular responses. Visual excitation and the phototransmission of light signals in the eye is controlled by cyclic-GMP regulated, Ca²⁺-specific channels. Because of the importance of cellular levels of cyclic nucleotides in mediating these various responses, regulating the synthesis and breakdown of cyclic nucleotides is an important matter. Thus adenylyl cyclase, which synthesizes cAMP from AMP, is activated to increase cAMP levels in muscle by binding of adrenaline to β-adrenergic receptors, while activation of guanylate cyclase and increased cGMP levels in photoreceptors leads to reopening of the Ca²⁺-specific channels and recovery of the dark state in the eye. There are nine known transmembrane isoforms of mammalian adenylyl cyclase, as well as a soluble form preferentially expressed in testis. Soluble adenylyl cyclase contains a P-loop, or nucleotide binding domain, and may be involved in male fertility (Buck, J. et al. (1999) Proc. Natl. Acad. Sci. USA 96:79-84).

In contrast, hydrolysis of cyclic nucleotides by cAMP and cGMP-specific phosphodiesterases (PDEs) produces the opposite of these and other effects mediated by increased cyclic nucleotide levels. PDEs appear to be particularly important in the regulation of cyclic nucleotides, considering the diversity found in this family of proteins. At least seven families of mammalian PDEs (PDE1-7) have been identified based on substrate specificity and affinity, sensitivity to cofactors, and sensitivity to inhibitory drugs (Beavo, J. A. (1995) Physiol. Rev. 75:725-748). PDE inhibitors have been found to be particularly useful in treating various clinical disorders. Rolipram, a specific inhibitor of PDE4, has been used in the treatment of depression, and similar inhibitors are undergoing evaluation as anti-inflammatory agents. Theophylline is a nonspecific PDE inhibitor used in the treatment of bronchial asthma and other respiratory diseases (Banner, K. H. and C. P. Page (1995) Eur. Respir. J. 8:996-1000).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

### Diseases Involving Cytoskeletal-Associated Proteins

Alzheimer's disease is a progressive neurodegenerative disorder that is characterized by the formation of senile plaques and neurofibrillary tangles containing amyloid beta peptide. These plaques are found in limbic and association cortices of the brain, including hippocampus, temporal cortices, cingulate cortex, amygdala, nucleus basalis and locus caeruleus. Early in Alzheimer's pathology, physiological changes are visible in the cingulate cortex (Minoshima, S. et al. (1997) Annals of Neurology 42:85-94). In subjects with advanced Alzheimer's disease, accumulating plaques damage the neuronal architecture in limbic areas and eventually cripple the memory process.

There are more than 180,000 new cases of breast cancer diagnosed each year, and the mortality rate for breast cancer approaches 10% of all deaths in females between the ages of 45-54 (K. Gish (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou C. M. et al. (2000) Natnre 406:747-752).

Breast cancer is a genetic disease commonly caused by mutations in breast epithelial cells. Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to noninherited mutations that occur in breast epithelial cells.

A good deal is already known about the expression of specific genes associated with breast cancer. For example, the relationship between expression of epidermal growth factor (EGF) and its receptor, EGFR, to human mammary carcinoma has been particularly well studied. (See Khazaie et al., supra, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed is human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou Z. et al. (1998) Int. J. Cancer 78:95-99; Chen, L. et al. (1990) Oncogene 5:1391-1395; Ulrix W. et al. (1999) FEBS Lett. 455:23-26; Sager, R. et al. (1996) Curr. Top. Microbio. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba II et al. (1998) Clin. Cancer. Res. 4:2931-2938). Such a model is particularly useful for comparing phenotypic and molecular characteristics of human mammary epithelial cells at various stages of malignant transformation.

Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of an lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda, R. et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen, S. et al. (2000; Int. J. Cancer, 86:512-517) used suppression subtractive hyoridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang, T. et al. (2000; Oncogene 19:1519-1528) used a combination of microarray analysis and subtractive hybridization to identify 17 genes differentially overexpresssed in squamous cell carcinoma compared with normal lung epithelium. Among the known genes they identified were keratin isoform 6, KOC, SPRC, IGFb2, connexin 26, plakofillin 1 and cytokeratin 13.

There is a need in the art for new compositions, including nucleic acids and proteins, for the diagnosis, prevention, and treatment of cell proliferative disorders, viral infections, and neurological disorders.

### SUMMARY OF THE INVENTION

Various embodiments of the invention provide purified polypeptides, structural and cytoskeleton-associated proteins, referred to collectively as “SCAP” and individually as “SCAP-1,” “SCAP-2,” “SCAP-3,” “SCAP-4,” “SCAP-5,” “SCAP-6,” “SCAP-7, ” “SCAP-8,” “SCAP-9,” “SCAP-10,” “SCAP-11,” “SCAP-12,” “SCAP-13,” “SCAP-14,” “SCAP-15,” “SCAP-16, “SCAP-17,” “SCAP-18,” “SCAP-19,” “SCAP-20,” “SCAP-21,” “SCAP-22,” “SCAP-23,” “SCAP-24,” and “SCAP-25,” and methods for using these proteins and their encoding polynucleotides for the detection, diagnosis, and treatment of diseases and medical conditions. Embodiments also provide methods for utilizing the purified structural and cytoskeleton-associated proteins and/or their encoding polynucleotides for facilitating the drug discovery process, including determination of efficacy, dosage, toxicity, and pharmacology. Related embodiments provide methods for utilizing the purified structural and cytoskeleton-associated proteins and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. Another embodiment provides an isolated polypeptide comprising an amino acid sequence of SEQ ID NO:1-25.

Still another embodiment provides an isolated polynucleotide encoding a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. In another embodiment, the polynucleotide encodes a polypeptide selected from the group consisting of SEQ ID NO:1-25. In an alternative embodiment, the polynucleotide is selected from the group consisting of SEQ ID NO:26-50.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from tie group consisting of SEQ ID NO:1-25, and d) an immunogenic fragment of a polypeptide having an amino acid sequence selected from the group consisting of SEQ ID NO:1-25. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25.

Still yet another embodiment provides an isolated polynucleotide selected from the group consisting of a) a polynucleotide comprising a polynucleotide sequence selected from the group consisting of SEQ ID NO:26-50, b) a polynucleotide comprising a naturally occurring polynucleotide sequence at least 90% identical or at least about 90% identical to a polynucleotide sequence selected from the group consisting of SEQ ID NO:26-50, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:26-50, b) a polynucleotide comprising a naturally occurring polynucleotide sequence at least 90% identical or at least about 90% identical to a polynucleotide sequence selected from the group consisting of SEQ ID NO:26-50, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:26-50, b) a polynucleotide comprising a naturally occurring polynucleotide sequence at least 90% identical or at least about 90% identical to a polynucleotide sequence selected from the group consisting of SEQ ID NO:26-50, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25, and a pharmaceutically acceptable excipient. In one embodiment, the composition can comprise an amino acid sequence selected from the group consisting of SEQ ID NO:1-25. Other embodiments provide a method of treating a disease or condition associated with decreased or abnormal expression of functional SCAP, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional SCAP, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional SCAP, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:26-50, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:26-50, ii) a polynucleotide comprising a naturally occurring polynucleotide sequence at least 90% identical or at least about 90% identical to a polynucleotide sequence selected from the group consisting of SEQ ID NO:26-50, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:26-50, ii) a polynucleotide comprising a naturally occurring polynucleotide sequence at least 90% identical or at least about 90% identical to a polynucleotide sequence selected from the group consisting of SEQ ID NO:26-50, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

### Definitions

“SCAP” refers to the amino acid sequences of substantially purified SCAP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of SCAP. Agonists may include proteins, nucleic acids, carbohydrates, small molecules, or any other compound or composition which modulates the activity of SCAP either by directly interacting with SCAP or by acting on components of the biological pathway in which SCAP participates.

An “allelic variant” is an alternative form of the gene encoding SCAP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding SCAP include those sequences with deletions, insertions, or substitutions of different nucleotides, resulting in a polypeptide the same as SCAP or a polypeptide with at least one functional characteristic of SCAP. Included within this definition are polymorphisms which may or may not be readily detectable using a particular oligonucleotide probe of the polynucleotide encoding SCAP, and improper or unexpected hybridization to allelic variants, with a locus other than the normal chromosomal locus for the polynucleotide encoding SCAP. The encoded protein may also be “altered,” and may contain deletions, insertions, or substitutions of amino acid residues which produce a silent change and result in a functionally equivalent SCAP. Deliberate amino acid substitutions may be made on the basis of one or more similarities in polarity, charge, solubility, hydrophobicity, hydrophilicity, and/or the amphipathic nature of the residues, as long as the biological or immunological activity of SCAP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of SCAP. Antagonists may include proteins such as antibodies, anticalins, nucleic acids, carbohydrates, small molecules, or any other compound or composition which modulates the activity of SCAP either by directly interacting with SCAP or by acting on components of the biological pathway in which SCAP participates.

The term “antibody” refers to intact immunoglobulin molecules as well as to fragments thereof, such as Fab, F(ab′)₂, and Fv fragments, which are capable of binding an epitopic determinant. Antibodies that bind SCAP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the inmune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide maybe replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers maybe specifically cross-linked to their cognate ligands, e.g., by photo-activation of a cross-linker. (See, e.g., Brody, E. N. and L. Gold (2000) J. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules maybe produced by any method including chemical synthesis or transcription. Once introduced into a cell, the complementary antisense molecule base-pairs with a nasally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic SCAP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding SCAP or fragments of SCAP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of SCAP or a polynucleotide encoding SCAP which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:26-50 can comprise a region of unique polynucleotide sequence that specifically identifies SEQ ID NO:26-50, for example, as distinct from any other sequence in the genome from which the fragment was obtained. A fragment of SEQ ID NO:26-50 can be employed in one or more embodiments of methods of the invention, for example, in hybridization and amplification technologies and in analogous methods that distinguish SEQ ID NO:26-50 from related polynucleotides. The precise length of a fragment of SEQ ID NO:26-50 and the region of SEQ ID NO:26-50 to which the fragment corresponds are routinely determinable by one of ordinary skill in the art based on the intended purpose for the fragment.

A fragment of SEQ ID NO:1-25 is encoded by a fragment of SEQ ID NO:26-50. A fragment of SEQ ID NO:1-25 can comprise a region of unique amino acid sequence that specifically identifies SEQ ID NO:1-25. For example, a fragment of SEQ ID NO:1-25 can be used as an immunogenic peptide for the development of antibodies that specifically recognize SEQ ID NO:1-25. The precise length of a fragment of SEQ ID NO:1-25 and the region of SEQ ID NO:1-25 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence. “Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default Percent identity is reported by CLUSTAL V as the “percent similarity” between aligned polynucleotide sequences.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, MD, and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr.-21-2000) set at default parameters. Such default parameters maybe, for example:

Matrix: BLOSUM62

Reward for match: 1

Penalty for mismatch: -2

Open Gap: 5 and Extension Gap: 2 penalties

Gap x drop-off 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr.-21-2000) with blastp set at default parameters. Such default parameters maybe, for example:

Matrix: BLOSUM62

Open Gap: 11 and Extension Gap: 1 penalties

Gap x drop-off 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of SCAP which is capable of eliciting an immune response when introduced into a living organism, for example, a mammal. The term “immunogenic fragment” also includes any polypeptide or oligopeptide fragment of SCAP which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of SCAP. For example, modulation may cause an increase or a decrease in protein activity, binding characteristics, or any other biological, functional, or immunological properties of SCAP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences maybe in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an SCAP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of SCAP.

“Probe” refers to nucleic acids encoding SCAP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, maybe used.

Methods for preparing and using probes and primers are described in the references, for example Sambrook, J. et al. (1989) Molecular Cloning: A Laboratory Manua, 2^{nd} ed., vol 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU, primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “misprinting library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing SCAP, nucleic acids encoding SCAP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant A splice variant may have, significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### The Invention

Various embodiments of the invention include new human structural and cytoskeleton-associated proteins (SCAP), the polynucleotides encoding SCAP, and the use of these compositions for the diagnosis, treatment, or prevention of cell proliferative disorders, viral infections, and neurological disorders.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are structural and cytoskeleton-associated proteins. For example, SEQ ID NO:6 is 95% identical, from residue M1 to residue T817, to rat neurabin II (GenBank ID g2853592) as determined by the Basic Local Alignment Search Tool (BLAST). (See Table 2.) The BLAST probability score is 0.0, which indicates the probability of obtaining the observed polypeptide sequence alignment by chance. SEQ ID NO:6 also contains a PDZ domain which binds ligands of transmembrane receptors, as determined by searching for statistically significant matches in the hidden Markov model (HMM)-based PFAM database of conserved protein family domains. (See Table 3.) Data from BLIMPS, MOTIFS, and BLAST analyses provide further corroborative evidence that SEQ ID NO:6 is a neurabin/spinophilin protein which plays an important role in linking the actin cytoskeleton to the plasma membrane. In an alternative example, SEQ ID NO:9 is 83% identical, from residue M1 to residue C766, to rat actin filament binding protein Frabin (GenBank ID g3342246) as determined by the Basic Local Alignment Search Tool (BLAST). (See Table 2.) The BLAST probability score is 0.0, which indicates the probability of obtaining the observed polypeptide sequence alignment by chance. SEQ ID NO:9 also contains FYVE zinc finger, PH, and RhoGEF domains as determined by searching for statistically significant matches in the is hidden Markov model (HMM)-based PFAM database of conserved protein family domains. (See Table 3.) Data from BLAST analyses provide further corroborative evidence that SEQ ID NO:9 is a cytoskeleton-associated protein. In an alternative example, SEQ ID NO:11 is 88% identical, from residue T8 to residue E414, to human cytokeratin 18 (GenBank ID g34037) as determined by the Basic Local Alignment Search Tool (BLAST). (See Table 2.) The BLAST probability score is 3.7e-158, which indicates the probability of obtaining the observed polypeptide sequence alignment by chance. SEQ ID NO:11 also contains an intermediate filament protein domain as determined by searching for statistically significant matches in the hidden Markov model (HMM)-based PFAM database of conserved protein family domains. (See Table 3.) Data from BUMPS, MOTIFS, and PROFILESCAN analyses provide further corroborative evidence that SEQ ID NO:11 is an intermediate filament protein In an alternative example, SEQ ID NO:13 is 57% identical, from residue P44 to residue D525 and 70% identical, from residue K584 to residue F648, to human actin-binding double zinc-finger protein (GenBank ID g2337952) as determined by the Basic Local Alignment Search Tool (BLAST). (See Table 2.) The BLAST probability score is 4.6e-172, which indicates the probability of obtaining the observed polypeptide sequence alignment by chance. SEQ ID NO:13 also contains LIM and villin domains as determined by searching for statistically significant matches in the hidden Markov model (HMM)-based PFAM database of conserved protein family domains. (See Table 3.) Data from MOTIFS, and PROFILESCAN analyses and BLAST analyses of the PRODOM and DOMO databases provide further corroborative evidence that SEQ ID NO:13 is a cytoskeletol-associated protein. In an alternative example, SEQ ID NO:16 is 23% identical, from residue A68 to residue V529, to human plakoglobin (GenBank ID g10334699) as determined by the Basic Local Alignment Search Tool (BLAST). (See Table 2.) The BLAST probability score is 1.3e-13, which indicates the probability of obtaining the observed polypeptide sequence alignment by chance. SEQ ID NO:16 also contains armadillo/beta-catenin-hike repeats, which are also found in plakoglobin, as determined by searching for statistically significant matches in the hidden Markov model (HMM)-based PFAM database of conserved protein family domains. (See Table 3.) Data from BLAST analysis of the DOMO database provides further corroborative evidence that SEQ ID NO:16 is a plakoglobin-like protein. In an alternative example, SEQ ID NO:20 is 93% identical, from residue M1 to residue S201, to murine scleraxis, a basic helix-loop-helix transcription factor (GenBank ID g998899) as determined by the Basic Local Alignment Search Tool (BLAST). (See Table 2.) The BLAST probability score is 2.5e-99, which indicates the probability of obtaining the observed polypeptide sequence alignment by chance. SEQ ID NO:20 also contains a helix-loop-helix DNA-binding domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:20 is a cytoskeleton-associated protein. SEQ ID NO:1-5, SEQ ID NO:7-8, SEQ ID NO:10, SEQ ID NO:12, SEQ ID NO:14-15, SEQ ID NO:17-19, and SEQ ID NO:21-25 were analyzed and annotated in a similar manner. The algorithms and parameters for the analysis of SEQ ID NO:1-25 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:26-50 or that distinguish between SEQ ID NO:26-50 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 maybe derived from the NCBI RefSeq Nucleotide Sequence Records Database (i.e., those sequences including the designation “NM” or “NT”) or the NCBI RefSeq Protein Sequence Records (Le., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) maybe used in place of the GenBank identifier (ie., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for example, |
| GFG, | GENSCAN (Stanford University, CA, USA) or FGENES |
| ENST | (Computer Genomics Group, The Sanger Centre, |
| | Cambridge, UK). |
| GBI | Hand-edited analysis of genomic sequences. |
| FL | Stitched or stretched genomic sequences (see Example V). |
| INCY | Full length transcript and exon prediction from mapping of EST |
| | sequences to the genome. Genomic location and EST |
| | composition data are combined to predict the exons |
| | and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

The invention also encompasses SCAP variants. A preferred SCAP variant is one which has at least about 80%, or alternatively at least about 90%, or even at least about 95% amino acid sequence identity to the SCAP amino acid sequence, and which contains at least one functional or structural characteristic of SCAP.

Various embodiments also encompass polynucleotides which encode SCAP. In a particular embodiment, the invention encompasses a polynucleotide sequence comprising a sequence selected from the group consisting of SEQ ID NO:26-50, which encodes SCAP. The polynucleotide sequences of SEQ ID NO:26-50, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding SCAP. In particular, such a variant polynucleotide will have at least about 70%, or alternatively at least about 85%, or even at least about 95% polynucleotide sequence identity to a polynucleotide encoding SCAP. A particular aspect of the invention encompasses a variant of a polynucleotide comprising a sequence selected from the group consisting of SEQ ID NO:26-50 which has at least about 70%, or alternatively at least about 85%, or even at least about 95% polynucleotide sequence identity to a nucleic acid sequence selected from the group consisting of SEQ ID NO:26-50. Any one of the polynucleotide variants described above can encode a polypeptide which contains at least one functional or structural characteristic of SCAP.

In addition, or in the alternative, a polynucleotide variant of the invention is a splice variant of a polynucleotide encoding SCAP. A splice variant may have portions which have significant sequence identity to a polynucleotide encoding SCAP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding SCAP over its entire length; however, portions of the splice variant will have at least about 70%, or alternatively at least about 85%, or alternatively at least about 95%, or alternatively 100% polynucleotide sequence identity to portions of the polynucleotide encoding SCAP. For example, a polynucleotide comprising a sequence of SEQ ID NO:49 is a splice variant of a polynucleotide comprising a sequence of SEQ ID NO:32 and a polynucleotide comprising a sequence of SEQ ID NO:50 is a splice variant of a polynucleotide comprising a sequence of SEQ ID NO:38. Any one of the splice variants described above can encode a polypeptide which contains at least one functional or structural characteristic of SCAP.

It win be appreciated by those skilled in the art that as a result of the degeneracy of the genetic code, a multitude of polynucleotide sequences encoding SCAP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring SCAP, and all such variations are to be considered as being specifically disclosed.

Although polynucleotides which encode SCAP and its variants are generally capable of hybridizing to polynucleotides encoding naturally occurring SCAP under appropriately selected conditions of stringency, it may be advantageous to produce polynucleotides encoding SCAP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host Other reasons for substantially altering the nucleotide sequence encoding SCAP and its derivatives without altering the encoded amino acid sequences include the production of RNA transcripts having more desirable properties, such as a greater half-life, than transcripts produced from the naturally occurring sequence.

The invention also encompasses production of polynucleotides which encode SCAP and SCAP derivatives, or fragments thereof, entirely by synthetic chemistry. After production, the synthetic polynucleotide may be inserted into any of the many available expression vectors and cell systems using reagents well known in the art Moreover, synthetic chemistry may be used to introduce mutations into a polynucleotide encoding SCAP or any fragment thereof

Embodiments of the invention can also include polynucleotides that are capable of hybridizing to the claimed polynucleotides, and, in particular, to those having the sequences shown in SEQ ID NO:26-50 and fragments thereof, under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and maybe used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acids encoding SCAP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences inhuman and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display maybe computer controlled. Capillary electrophoresis is especially preferable for sequencing small DNA fragments which maybe present in limited amounts in a particular sample.

In another embodiment of the invention, polynucleotides or fragments thereof which encode SCAP may be cloned in recombinant DNA molecules that direct expression of SCAP, or fragments or functional equivalents thereof, in appropriate host cells. Due to the inherent degeneracy of the genetic code, other polynucleotides which encode substantially the same or a functionally equivalent polypeptides may be produced and used to express SCAP.

The polynucleotides of the invention can be engineered using methods generally known in the art in order to alter SCAP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis maybe used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotecbnol. 17:259-264; and Crameri, A. et al. (1996) Nat. Biotechnol. 14:315-319) to alter or improve the biological properties of SCAP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding SCAP maybe synthesized, in whole or in part, using one or more chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, SCAP itself or a fragment thereof may be synthesized using chemical methods known in the art. For example, peptide synthesis can be performed using various solution-phase or solid-phase techniques. (See, e.g., Creighton, T. (1984) Proteins, Structures and Molecular Properties, WH Freeman, New York N.Y., pp. 55-60; and Roberge, J. Y. et al. (1995) Science 269:202-204.) Automated synthesis maybe achieved using the ABI 431A peptide synthesizer (Applied Biosystems). Additionally, the amino acid sequence of SCAP, or any part thereof, maybe altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active SCAP, the polynucleotides encoding SCAP or derivatives thereof maybe inserted into an appropriate expression vector, i.e., a vector which contains the necessary elements for transcriptional and translational control of the inserted coding sequence in a suitable host These elements include regulatory sequences, such as enhancers, constitutive and inducible promoters, and 5′ and 3′ untranslated regions in the vector and in polynucleotides encoding SCAP. Such elements may vary in their strength and specificity. Specific initiation signals may also be used to achieve more efficient translation of polynucleotides encoding SCAP. Such signals include the ATG initiation codon and adjacent sequences, e.g. the Kozak sequence. In cases where a polynucleotide sequence encoding SCAP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression, vectors containing polynucleotides encoding SCAP and appropriate transcriptional and translational control elements. These methods include in vitro recombinant DNA techniques, synthetic techniques, and in vivo genetic recombination. (See, e.g., Sambrook, J. et al. (1989) Molecular Cloning. A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express polynucleotides encoding SCAP. These include, but are not limited to, microorganism& such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of polynucleotides to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M N. Somia (1997) Nature 389:239-242.) The invention is not limited by the host cell employed.

In bacterial systems, a number of cloning and expression vectors may be selected depending upon the use intended for polynucleotides encoding SCAP. For example, routine cloning, subcloning, and propagation of polynucleotides encoding SCAP can be achieved using a multifunctional E. coli vector such as PBLUESCRIPT (Stratagene, La Jolla Calif.) or PSPORT1 plasmid Invitrogen). Ligation of polynucleotides encoding SCAP into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of SCAP are needed, e.g. for the production of antibodies, vectors which direct high level expression of SCAP may be used. For example, vectors containing the strong, inducible SP6 or T7 bacteriophage promoter may be used.

Yeast expression systems may be used for production of SCAP. A number of vectors containing constitutive or inducible promoters, such as alpha factor, alcohol oxidase, and PGH promoters, maybe used in the yeast Saccharomyces cerevisiae or Pichia pastoris. In addition, such vectors direct either the secretion or intracellular retention of expressed proteins and enable integration of foreign polynucleotide sequences into the host genome for stable propagation (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of SCAP. Transcription of polynucleotides encoding SCAP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters maybe used. (See, e.g., Coruzzi, G. et al. (1984) IMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, polynucleotides encoding SCAP maybe ligated into an adenovirus transcription/translation complex consisting of the late promoter and tripartite leader sequence. Insertion in a non-essential E1 or E3 region of the viral genome may be used to obtain infective virus which expresses SCAP in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of SCAP in cell lines is preferred. For example, polynucleotides encoding SCAP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding SCAP is inserted within a marker gene sequence, transformed cells containing polynucleotides encoding SCAP can be identified by the absence of marker gene function. Alternatively, a marker gene can be placed in tandem with a sequence encoding SCAP under the control of a single promoter. Expression of the marker gene in response to induction or selection usually indicates expression of the tandem gene as well.

In general, host cells that contain the polynucleotide encoding SCAP and that express SCAP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of SCAP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on SCAP is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding SCAP include oligolabeling, nick translation, end-labeling, or PCR amplification using a labeled nucleotide. Alternatively, polynucleotides encoding SCAP, or any fragments thereof, maybe cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding SCAP may be cultured under conditions suitable for the expression and recovery of the protein from cell culture. The protein produced by a transformed cell maybe secreted or retained intracellularly depending on the sequence and/or the vector used. As will be understood by those of skill in the art, expression vectors containing polynucleotides which encode SCAP may be designed to contain signal sequences which direct secretion of SCAP through a prokaryotic or eukaryotic cell membrane.

In addition, a host cell strain maybe chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding SCAP may be ligated to a heterologous sequence resulting in translation of a fusion protein in any of the aforementioned-host systems. For example, a chimeric SCAP protein containing a heterologous moiety that can be recognized by a commercially available antibody may facilitate the screening of peptide libraries for inhibitors of SCAP activity. Heterologous protein and peptide moieties may also facilitate purification of fusion proteins using commercially available affinity matrices. Such moieties include, but are not limited to, glutathione S-transferase (GST), maltose binding protein MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the SCAP encoding sequence and the heterologous protein sequence, so that SCAP may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In another embodiment, synthesis of radiolabeled SCAP maybe achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

SCAP, fragments of SCAP, or variants of SCAP may be used to screen for compounds that specifically bind to SCAP. One or more test compounds may be screened for specific binding to SCAP. In various embodiments, 1, 2, 3, 4, 5, 10, 20, 50, 100, or 200 test compounds can be screened for specific binding to SCAP. Examples of test compounds can include antibodies, anticalins, oligonucleotides, proteins (e.g., ligands or receptors), or small molecules.

In related embodiments, variants of SCAP can be used to screen for binding of test compounds, such as antibodies, to SCAP, a variant of SCAP, or a combination of SCAP and/or one or more variants SCAP. In an embodiment, a variant of SCAP can be used to screen for compounds that bind to a variant of SCAP, but not to SCAP having the exact sequence of a sequence of SEQ ID NO:1-25. SCAP variants used to perform such screening can have a range of about 50% to about 99% sequence identity to SCAP, with various embodiments having 60%, 70%, 75%, 80%, 85%, 90%, and 95% sequence identity.

In an embodiment, a compound identified in a screen for specific binding to SCAP can be closely related to the natural ligand of SCAP, e.g., a ligand or fragment thereof, a natural substrate, a structural or functional mimetic, or a natural binding partner. (See, e.g., Coligan, J. E. et al. (1991) Current Protocols in Immunology 1(2):Chapter 5.) In another embodiment, the compound thus identified can be a natural ligand of a receptor SCAP. (See, e.g., Howard, A. D. et al. (2001) Trends Pharmacol. Sci.22:132-140; Wise, A. et al. (2002) Drug Discovery Today 7:235-246.)

In other embodiments, a compound identified in a screen for specific binding to SCAP can be closely related to the natural receptor to which SCAP binds, at least a fragment of the receptor, or a fragment of the receptor including all or a portion of the ligand binding site or binding pocket For example, the compound maybe a receptor for SCAP which is capable of propagating a signal, or a decoy receptor for SCAP which is not capable of propagating a signal (Asbkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Imnunex Corp., Seattle Wash.), which is efficacious for treating rheumatoid arthritis in humans. Etanercept is an engineered p75 tumor necrosis factor (TFN) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to SCAP, fragments of SCAP, or variants of SCAP. The binding specificity of the antibodies thus screened can thereby be selected to identify particular fragments or variants of SCAP. In one embodiment, an antibody can be selected such that its binding specificity allows for preferential identification of specific fragments or variants of SCAP. In another embodiment, an antibody can be selected such that its binding specificity allows for preferential diagnosis of a specific disease or condition having-increased, decreased, or otherwise abnormal production of SCAP.

In an embodiment, anticalins can be screened for specific binding to SCAP, fragments of SCAP, or variants of SCAP. Anticalins are ligand-binding proteins that have been constructed based on a lipocalin scaffold (Weiss, G. A. and H. B. Lowman (2000) Chem. Biol. 7-R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit SCAP involves producing appropriate cells which express SCAP, either as a secreted protein or on the cell membrane. Preferred cells include cells from mammals, yeast, Drosophila, or E. coli. Cells expressing SCAP or cell membrane fractions which contain SCAP are then contacted with a test compound and binding, stimulation, or inhibition of activity of either SCAP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with SCAP, either in solution or affixed to a solid support, and detecting the binding of SCAP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands. (See, e.g., Matthews, D. J. and J. A. Wells. (1994) Chem. Biol. 1:25-30.) In another related embodiment, one or more amino acid substitutions can be introduced into a polypeptide compound (such as a ligand) to improve or alter its ability to bind to its natural receptors. (See, e.g., Cunningham, B. C. and J. A. Wells (1991) Proc. Natl. Acad. Sci. USA 88:3407-3411; Lowman, H. B. et al. (1991) J. Biol Chem. 266:10982-10988.)

SCAP, fragments of SCAP, or variants of SCAP maybe used to screen for compounds that modulate the activity of SCAP. Such compounds may include agonists, antagonists, or partial or inverse agonists. In one embodiment, an assay is performed under conditions permissive for SCAP activity, wherein SCAP is combined with at least one test compound, and the activity of SCAP in the presence of a test compound is compared with the activity of SCAP in the absence of the test compound. A change in the activity of SCAP in the presence of the test compound is indicative of a compound that modulates the activity of SCAP. Alternatively, a test compound is combined with an in vitro or cell-free system comprising SCAP under conditions suitable for SCAP activity, and the assay is performed. In either of these assays, a test compound which modulates the activity of SCAP may do so indirectly and need not come in direct contact with the test compound. At least one and up to a plurality of test compounds may be screened.

In another embodiment, polynucleotides encoding SCAP or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding SCAP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding SCAP can also be used to create “knockin” humanized animals (pigs) or transgenic animals (mice or rats) to model human disease. With knockin technology, a region of a polynucleotide encoding SCAP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress SCAP, e.g., by secreting SCAP in its milk, may also serve as a convenient source of that protein (Janne, J. et al. (1998) Biotechnol. Annu. Rev. 4:55-74).

### Therapeutics

Chemical and structural similarity, e.g., in the context of sequences and motifs, exists between regions of SCAP and structural and cytoskeleton-associated proteins. In addition, examples of tissues expressing SCAP can be found in Table 6 and can also be found in Example XI. Therefore, SCAP appears to play a role in cell proliferative disorders, viral infections, and neurological disorders. In the treatment of disorders associated with increased SCAP expression or activity, it is desirable to decrease the expression or activity of SCAP. In the treatment of disorders associated with decreased SCAP expression or activity, it is desirable to increase the expression or activity of SCAP.

Therefore, in one embodiment, SCAP or a fragment or derivative thereof maybe administered to a subject to treat or prevent a disorder associated with decreased expression or activity of SCAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuxia, polycythemia vera, psoriasis, primary thrombocythemia, and a cancer including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a viral infection such as those caused by adenoviruses (acute respiratory disease, pneumonia), arenaviruses (lymphocytic choriomeningitis), bunyaviruses (Hantavirus), coronaviruses (pneumonia, chronic bronchitis), hepadnaviruses (hepatitis), herpesviruses herpes simplex virus, varicella-zoster virus, Epstein-Barr virus, cytomegalovirus), flaviviruses (yellow fever), orthomyxoviruses (influenza), papillomaviruses (cancer), paramyxoviruses (measles, mumps), picornoviruses (rhinovirus, poliovirus, coxsackie-virus), polyomaviruses (OK virus, JC virus), poxviruses (smallpox), reovirus (Colorado tick fever), retroviruses (human immunodeficiency virus, human T lymphotropic virus), rhabdoviruses (rabies), rotaviruses (gastroenteritis), and togaviruses (encephalitis, rubella); and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, a prion disease including kuru, Creutzfeldt-Jakob disease, and Gerstrnann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, and Tourette's disorder.

In another embodiment, a vector capable of expressing SCAP or a fragment or derivative thereof maybe administered to a subject to treat or prevent a disorder associated with decreased expression or activity of SCAP including, but not limited to, those described above.

In a further embodiment, a composition comprising a substantially purified SCAP in conjunction with a suitable pharmaceutical carrier may be administered to a subject to treat or prevent a disorder associated with decreased expression or activity of SCAP including, but not limited to, those provided above.

In still another embodiment, an agonist which modulates the activity of SCAP maybe administered to a subject to treat or prevent a disorder associated with decreased expression or activity of SCAP including, but not limited to, those listed above.

In a further embodiment, an antagonist of SCAP may be administered to a subject to treat or prevent a disorder associated with increased expression or activity of SCAP. Examples of such disorders include, but are not limited to, those cell proliferative disorders, viral infections, and neurological disorders described above. In one aspect, an antibody which specifically binds SCAP may be used directly as an antagonist or indirectly as a targeting or delivery mechanism for bringing a pharmaceutical agent to cells or tissues which express SCAP.

In an additional embodiment, a vector expressing the complement of the polynucleotide encoding SCAP maybe administered to a subject to treat or prevent a disorder associated with increased expression or activity of SCAP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of SCAP may be produced using methods which are generally known in the art. In particular, purified SCAP may be used to produce antibodies or to screen libraries of pharmaceutical agents to identify those which specifically bind SCAP. Antibodies to SCAP may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with SCAP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH and dinitrophenol. Among adjuvants used in humans, BCG (bacilli Calmette-Guerin) and Corynebacterium parvum are especially preferable.

It is preferred that the oligopeptides, peptides, or fragments used to induce antibodies to SCAP have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of SCAP amino acids may be fused with those of another protein, such as KLH, and antibodies to the chimeric molecule may be produced.

Monoclonal antibodies to SCAP maybe prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce SCAP-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for SCAP may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art Such immunoassays typically involve the measurement of complex formation between SCAP and its specific antibody. A two-site, monoclonal-based immunoassay utilizing monoclonal antibodies reactive to two non-interfering SCAP epitopes is generally used, but a competitive binding assay may also be employed (Pound, supra).

Various methods such as Scatchard analysis in conjunction with radioimmunoassay techniques may be used to assess the affinity of antibodies for SCAP. Affinity is expressed as an association constant, Kₐ, which is defined as the molar concentration of SCAP-antibody complex divided by the molar concentrations of free antigen and free antibody under equilibrium conditions. The Kₐ determined for a preparation of polyclonal antibodies, which are heterogeneous in their affinities for multiple SCAP epitopes, represents the average affinity, or avidity, of the antibodies for SCAP. The Kₐ determined for a preparation of monoclonal antibodies, which are monospecific for a particular SCAP epitope, represents a true measure of affinity. High-affinity antibody preparations with Kₐ ranging from about 10⁹ to 10¹² L/mole are preferred for use in immunoassays in which the SCAP-antibody complex must withstand rigorous manipulations. Low-affinity antibody preparations with Kₐ ranging from about 10⁶ to 10⁷ L/mole are preferred for use in immunopurification and similar procedures which ultimately require dissociation of SCAP, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, john Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of SCAP-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, polynucleotides encoding SCAP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding SCAP. Such technology is well known in the art, and antisense oligonucleotides or larger fragments can be designed from various locations along the coding or control regions of sequences encoding SCAP. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (See, e.g., Slater, J. E. et al. (1998) J. Allergy Clin. Immunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art (See, e.g., Rossi, J. J. (1995) Br. Med. Bull 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding SCAP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltirnore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoyna cruzi). In the case where a genetic deficiency in SCAP expression or regulation causes disease, the expression of SCAP from an appropriate population of transduced cells may alleviate the clinical manifestations caused by the genetic deficiency.

In a further embodiment of the invention, diseases or disorders caused by deficiencies in SCAP are treated by constructing mammalian expression vectors encoding SCAP and introducing these vectors by mechanical means into SCAP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Récipon (1998) Curr. Opin. Biotechnol 9:445-450).

Expression vectors that may be effective for the expression of SCAP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). SCAP maybe expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, P. M. V. and H. M. Blau, supra)), or (iii) a tissue-specific promoter or the native promoter of the endogenous gene encoding SCAP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method. (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to SCAP expression are treated by constructing a retrovirus vector consisting of (i) the polynucleotide encoding SCAP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding SCAP to cells which have one or more genetic abnormalities with respect to the expression of SCAP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another embodiment, a herpes-based, gene therapy delivery system is used to deliver polynucleotides encoding SCAP to target cells which have one or more genetic abnormalities with respect to the expression of SCAP. The use of herpes simplex virus (HSV)-based vectors may be especially valuable for introducing SCAP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding SCAP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenonic RNA replicates to higher levels than the full length genomic RNA, resulting in the overproduction of capsid proteins relative to the viral proteins with enzymatic activity (e.g., protease and polymerase). Similarly, inserting the coding sequence for SCAP into the alphavirus genome in place of the capsid-coding region results in the production of a large number of SCAP-coding RNAs and the synthesis of high levels of SCAP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of SCAP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. B. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding SCAP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding SCAP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding SCAP. Compounds which may be effective in altering expression of a specific polynucleotide may include, but are not limited to, ohigonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either hiihibitors or promoters of polynucleotide expression. Thus, in the treatment of disorders associated with increased SCAP expression or activity, a compound which specifically inhibits expression of the polynucleotide encoding SCAP may be therapeutically useful, and in the treatment of disorders associated with decreased SCAP expression or activity, a compound which specifically promotes expression of the polynucleotide encoding SCAP may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occu ng or non-natural chemical compounds; rational design of a compound based on chemical and/or structural properties of the target polynucleotide; and selection from a library of chemical compounds created combinatorially or randomly. A sample comprising a polynucleotide encoding SCAP is exposed to at least one test compound thus obtained. The sample may comprise, for example, an intact or permeabilized cell, or an in vitro cell-free or reconstituted biochemical system. Alterations in the expression of a polynucleotide encoding SCAP are assayed by any method commonly known in the art. Typically, the expression of a specific nucleotide is detected by hybridization with a probe having a nucleotide sequence complementary to the sequence of the polynucleotide encoding SCAP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schitizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biocheni Biophys. Res. Commun. 268:8-13). A particular embodiment of the present invention involves screening a combinatorial library of oligonucleotides (such as deoxyribonucleotides, nbonucleotides, peptide nucleic acids, and modified oligonucleotides) for antisense activity against a specific polynucleotide sequence (Bruice, T. W. et al. (1997) U.S. Pat. No.5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of SCAP, antibodies to SCAP, and mimetics, agonists, antagonists, or inhibitors of SCAP.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising SCAP or fragments thereof. For example, liposome preparations containing a cell-impermeable macromolecule may promote cell fusion and intracellular delivery of the macromolecule. Alternatively, SCAP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example SCAP or fragments thereof, antibodies of SCAP, and agonists, antagonists or inhibitors of SCAP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the ID₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### Diagnostics

In another embodiment, antibodies which specifically bind SCAP may be used for the diagnosis of disorders characterized by expression of SCAP, or in assays to monitor patients being treated with SCAP or agonists, antagonists, or inhibitors of SCAP. Antibodies useful for diagnostic purposes may be prepared in the same manner as described above for therapeutics. Diagnostic assays for SCAP include methods which utilize the antibody and a label to detect SCAP in human body fluids or in extracts of cells or tissues. The antibodies may be used with or without modification, and may be labeled by covalent or non-covalent attachment of a reporter molecule. A wide variety of reporter molecules, several of which are described above, are known in the art and maybe used.

A variety of protocols for measuring SCAP, including ELISAs, RIAs, and FACS, are known in the art and provide a basis for diagnosing altered or abnormal levels of SCAP expression. Normal or standard values for SCAP expression are established by combining body fluids or cell extracts taken from normal mammalian subjects, for example, human subjects, with antibodies to SCAP under conditions suitable for complex formation. The amount of standard complex formation may be quantitated by various methods, such as photometric means. Quantities of SCAP expressed in subject, control, and disease samples from biopsied tissues are compared with the standard values. Deviation between standard and subject values establishes the parameters for diagnosing disease.

In another embodiment of the invention, polynucleotides encoding SCAP maybe used for diagnostic purposes. The polynucleotides which maybe used include oligonucleotides, complementary RNA and DNA molecules, and PNAs. The polynucleotides may be used to detect and quantify gene expression in biopsied tissues in which expression of SCAP may be correlated with disease. The diagnostic assay may be used to determine absence, presence, and excess expression of SCAP, and to monitor regulation of SCAP levels during therapeutic intervention.

In one aspect, hybridization with PCR probes which are capable of detecting polynucleotides, including genomic sequences, encoding SCAP or closely related molecules may be used to identify nucleic acid sequences which encode SCAP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding SCAP, allelic variants, or related sequences.

Probes may also be used for the detection of related sequences, and may have at least 50% sequence identity to any of the SCAP encoding sequences. The hybridization probes of the subject invention may be DNA or RNA and may be derived from the sequence of SEQ ID NO:26-50 or from genomic sequences including promoters, enhancers, and introns of the SCAP gene.

Means for producing specific hybridization probes for polynucleotides encoding SCAP include the cloning of polynucleotides encoding SCAP or SCAP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding SCAP may be used for the diagnosis of disorders associated with expression of SCAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and a cancer including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a viral infection such as those caused by adenoviruses (acute respiratory disease, pneumonia), arenaviruses (lymphocytic choriomeningitis), bunyaviruses (Hantavirus), coronaviruses (pneumonia, chronic bronchitis), hepadnaviruses (hepatitis), herpesviruses (herpes simplex virus, varicella-zoster virus, Epstein-Barr virus, cytomegalovirus), flaviviruses (yellow fever), orthomyxoviruses (influenza), papillomaviruses (cancer), paramyxoviruses (measles, mumps), picornoviruses (rhinovirus, poliovirus, coxsackie-virus), polyomaviruses (BK virus, JC virus), poxvirises (smallpox), reovirus (Colorado tick fever), retroviruses (human immunodeficiency virus, human T lymphotropic virus), rhabdoviruses (rabies), rotaviruses (gastroenteritis), and togaviruses (encephalitis, rubella); and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, a prion disease including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system-disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, and Tourette's disorder. Polynucleotides encoding SCAP may be used in Southern or northern analysis, dot blot, or other membrane-based technologies; in PCR technologies; in dipstick, pin, and multiformat ELISA-like assays; and in microarrays utilizing fluids or tissues from patients to detect altered SCAP expression. Such qualitative or quantitative methods are well known in the art.

In a particular aspect, polynucleotides encoding SCAP may be used in assays that detect the presence of associated disorders, particularly those mentioned above. Polynucleotides complementary to sequences encoding SCAP may be labeled by standard methods and added to a flid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding SCAP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of SCAP, a normal or standard profile for expression is established. This may be accomplished by combining body fluids or cell extracts taken from normal subjects, either animal or human, with a sequence, or a fragment thereof, encoding SCAP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding SCAP may involve the use of PCR. These oligomers may be chemically synthesized, generated enzymatically, or produced in vitro. Oligomers will preferably contain a fragment of a polynucleotide encoding SCAP, or a fragment of a polynucleotide complementary to the polynucleotide encoding SCAP, and will be employed under optimized conditions for identification of a specific gene or condition Oligomers may also be employed under less stringent conditions for detection or quantification of closely related DNA or RNA sequences.

In a particular aspect, oligonucleotide primers derived from polynucleotides encoding SCAP maybe used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding SCAP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifng polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs maybe used to study the genetic basis of human disease. For example, at least 16 common SNPs have been associated with non-insulin-dependent diabetes mellitus. SNPs are also useful for examing differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641.)

Methods which may also be used to quantity the expression of SCAP include radiolabeling or biotinylating nucleotides, coamplification of a control nucleic acid, and interpolating results from standard curves. (See, e.g., Melby, P. C. et al. (1993) J. Inmunol. Methods 159:235-244; Duplaa, C. et al. (1993) Anal Biochem. 212:229-236.) The speed of quantitation of multiple samples maybe accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, SCAP, fragments of SCAP, or antibodies specific for SCAP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occuning environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatmnent with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released February 29, 2000, available at http://www.niehs.nih.gov/oc/news/toxchip.htm.) Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in, an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visali in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for SCAP to quantify the levels of SCAP expression. In one embodiment, the antibodies are used as elements on a microarray, and protein expression levels are quantified by exposing the microarray to the sample and detecting the levels of protein bound to each array element (Lueking, A. et al. (1999) Anal Biochem. 270:103-111; Mendoze, L. G. et a (1999) Biotechniques 27:778-788). Detection maybe performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays maybe prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application W095/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrams: A Practical Approach, M. Schena, ed. (1999) Oxford University Press, London.

In another embodiment of the invention, nucleic acid sequences encoding SCAP may be used to generate hybridization probes useful in mapping the naturally occurring genomic sequence. Either coding or noncoding sequences maybe used, and in some instances, noncoding sequences maybe preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e;g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences may be used to develop genetic linkage maps, for example, which correlate the inheritance of a disease state with the inheritance of a particular chromosome region or restriction fragment length polymorphism (RAPL). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding SCAP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, SCAP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between SCAP and the agent being tested maybe measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with SCAP, or fragments thereof, and washed. Bound SCAP is then detected by methods well known in the art. Purified SCAP can also be coated directly onto plates for use in the aforementioned drug screening techniques. Alternatively, non-neutralizing antibodies can be used to capture the peptide and immobilize it on a solid support

In another embodiment, one may use competitive drug screening assays in which neutrazing antibodies capable of binding SCAP specifically compete with a test compound for binding SCAP. In this manner, antibodies can be used to detect the presence of any peptide which shares one or more antigenic determinants with SCAP.

In additional embodiments, the nucleotide sequences which encode SCAP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, mentioned above and below, including U.S. Ser. No. 60/296,865, U.S. Ser. No. 60/298,664, U.S. Ser. No. 60/300,149, U.S. Ser. No. 60/302,340, U.S. Ser. No. 60/303,481, US. Ser. No. 60/305,059, U.S. Ser. No. 60/343,557, and U.S. Ser. No. 60/296,878 are expressly incorporated by reference herein.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL Invitrogen), a monophasic solution of phenol and guanidine isothiocyanate. The resulting lysates were centrifuged over CsCl cushions or extracted with chloroform RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-Blue, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Harilton) liquid transfer system. CDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masldng ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhtabditis elegans, Saccharomyces cerevisiae, Schizosaccharomyces pombe, and Candida albicans (Incyte Genomics, Palo Alto Calif.); bidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:26-50. Fragments from about 20 to about 4000 nucleotides which are useful in hybridization and amplification technologies are described in Table 4, column 2.

IV. Identification and Editing of Coding Sequences from Genornic DNA

Putative structural and cytoskeleton-associated proteins were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode structural and cytoskeleton-associated proteins, the encoded polypeptides were analyzed by querying against PFAM models for structural and cytoskeleton-associated proteins. Potential structural and cytoskeleton-associated proteins were also identified by homology to Incyte cDNA sequences that had been annotated as structural and cytoskeleton-associated proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

Stretched” Sequences

Partial DNA sequences were extended to full length with an algorithm based on BLAST analysis. First, partial cDNAs assembled as described in Example m were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of SCAP Encoding Polynucleotides

The sequences which were used to assemble SEQ ID NO:26-50 were compared with sequences from the Incyte LIFESEQ database and public domain databases using BLAST and other implementations of the Smith-Waterman algorithm. Sequences from these databases that matched SEQ ID NO:26-50 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm (The centimorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBa&k or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding SCAP are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding SCAP. cDNA sequences and cDNA library/tissue information are found in the LIFESEQ GOLD database (Incyte Genomics, Palo Alto Calif.).

VIII. Extension of SCAP Encoding Polynucleotides

Full length polynucleotides are produced by extension of an appropriate fragment of the full length molecule using oligonucleotide primers designed from this fragment One primer was synthesized to initiate 5′ extension of the known fragment, and the other primer was synthesized to initiate 3′ extension of the known fragment The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polyrnerase (Stratagene), with the following parameters for primer pair PCI A and PCI B: Step 1: 94° C., 3 min; Step 2: 94° C., 15 see; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1X TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2x carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in SCAP Encoding Polynucleotides

Common DNA sequence variants known as single nucleotide polymorpbisms (SNPs) were identified in SEQ ID NO:26-50 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minum PFhred quality score of 15, and removed sequence alignment errors and errors resulting from improper triming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asia Allele frequencies were first analyzed in the Caucasian population; in some cases those SNPs which showed no allelic variance in this population were not further tested in the other three populations.

X. Labeling and use of Individual Hybridization Probes

Hybridization probes derived from SEQ ID NO:26-50 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ−³²P] adenosine triphosphate (Amersham Biosciences), and T4 polynucleotide kie (DuPont NEN, Boston Mass.). The labeled oligonucleotides are substantially purified using a SEPHADEX G-25 superfine size exclusion dexaaan bead column (Amersharn Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualied using autoradiography or an alternative imaging means and compared.

XM. Microarrays

The linkage or synthesis of array elements upon a microarray can be achieved utilizing photolithography, piezoelectric printing (ink-jet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element Alternatively, laser desorbtion and mass spectrometry may be used for detection of hybridization. The degree of comnplementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1×first strand buffer, 0.03 units/μl RNase inhibitor, 500 μM DATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GENBRIGHT kits (Incyte). Specific control poly(A)⁺ RNAs are synthesized by int vitro transcription from non-coding yeast genomic DNA. After incubation at 37° C. for 2 hr, each reaction sample (one with Cy3 and another with Cy5 labeling) is treated with 2.5 ml of 0.5M sodiumhydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA inset Array elements are amplified in thity cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Micro arrays are UV-crosslinked using a STRATAUNER IUV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μt of sample mixture consisting of 0.2 μg each of Cy3 and Cy5 labeled cDNA synthesis products in 5×SSC, 0.2% SDS hybridization buffer. The sample mure is heated to 650° C. for 5 minutes and is aliquoted onto the microarray surface and covered with an 1.8 cm² coverslip. The arrays are transferred to a waterproof chamber having a cavityjust slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first washbuffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectr

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibted at least about a two-fold change in expression, a signal-to-background ratio of at least 2.5, and an element spot size of at least 40% were identified as differentialy expressed using the GEMTOOLS program (Incyte Genomics).

Expression

Microarray analyses were performed on early confluent cultures of the human cell line C3C, a clonal derivative of the Hep G2 hepatoma cell line isolated from a 15-year old male with liver tumor, which was selected for its strong contact growth inhibition. The use of a clonal population enhances the reproducibility of the results. By maintaining a regularly monitored Master Bank and performing limited passages of cells, the robustness of the cell line for long-term study is greatly enhanced. These cells were used to evaluate the expression of SCAP in response to a steroidal compound. Additional Inicroarray experiments were performed comparing gene expression in nonmalignant human breast primary epithelial cells to that of various carcinoma lines at different stages of tumor progression. This cross-comparison protocol evaluated differential expression profiles in human HMEC cells (a primary, non-tumorigenic breast epithelial cell line isolated from a normal donor) as opposed to MCF7 (a nonmalignant breast adenocarcinoma cell line isolated from the pleural effusion of a 69-year old female), Sk-BR-3 (a breast adenocarcinoma cell line isolated from a malignant pleural effusion of a 43-year old female), MDA-mb-231 (a breast tumor cell line isolated from the pleural effusion of a 51-year old female), and MDA-mb-435S (a spindle-shaped strain that evolved from the parent line (435) as isolated in 1976 from the pleural effusion of a 31-year old female with metastatic, ductal adenocarcinoma) cells. The cells were grown in the supplier's recommended medium to 70-80% confluence prior to RNA harvest. Cells were lysed in Trizol and total RNA fraction was recovered according to manufacturer's protocols. Poly(A) mRNA was purified using standard oligo-dT selection methods. Cy3 and Cy5 probes were prepared according to the standard operating procedure developed at Incyte's raicroarray facility.

Response to Steroids:

SEQ ID NO:33 showed differential expression in response to treatment with a steroid as determined by microarray analysis. The expression of SEQ ID NO:33 was decreased by at least 2-fold in early confluent human C3C cells in response to treatment with 1, 10, and 100 μM of beclomethasone for 1, 3, and 6 h. Beclomethasone is a synthetic glucocorticoid that is used for treating steroid-dependent asthma, relieving symptoms associated with allergic or nonallergic (vasomotor) rhinitis, or for preventing recurring nasal polyps following surgical removal. Glucocorticoids are naturally occurring hormones that prevent or suppress inflammation and immune responses when administered at pharmacological doses.

Tumor Versus Normal Response

In an alternative example, SEQ ID NO:34 showed differential expression in several tumor cell lines compared to the normal human breast epithelial cell line HMEC as determined by microarray analysis. The expression of SEQ ID NO:34 was decreased by at least 2-fold in four different breast tumor cell lines which were harvested from donors with breast cancer at various stages of tumor progression.

In an alternative example, the expression of SEQ ID NO:37, as determined by microarray analysis, was decreased by at least two fold in breast tumor tissues relative to normal breast tissues. The breast tumor tissues were harvested from a 43 year old female donor diagnosed with invasive lobular carcinoma. The tumor is well differentiated and metastatic. The normal breast tissues were harvested from grossly uninvolved breast tissue of the same donor. Therefore, SEQ ID NO:37 is useful in diagnostic assays for breast cancer.

In another example, the expression of SEQ ID NO:37 was decreased by at least two fold in a prostate carcinoma cell line relative to normal prostate epithelial cells. The prostate carcinoma cell line was isolated from a metastatic site in the brain of a 69 year old male with widespread metastatic prostate carcinoma, and the prostate epithelial cell line was isolated from a normal donor. Therefore, SEQ ID NO:37 is useful in diagnostic assays for prostate cancer.

In an alternative example, SEQ ID NO:41 showed differential expression in brain cingulate from a patient with Alzheimer's disease compared to matched microscopically normal tissue from the same donor as determined by microarray analysis. The expression of SCAP-16 was increased at least two-fold in cingulate tissue with Aeimer-s disease. Therefore, SEQ ID NO:41 is useful in diagnostic assays for neurological disorders, including Alzheimer's disease.

In an alternative example, SEQ ID NO:42 showed differential expression in breast tissue from a patient with cancer compared to matched microscopically normal tissue from the same donor as determined by microarray analysis. The expression of SCAP-17 was decreased at least two-fold in cancerous breast tissue. SEQ ID NO:42 also showed differential expression in the human mammary gland cell line MCF-l10A compared to breast carcinoma cell lines, MCF7, BT-20, T-47D, Sk-BR-3, MDA-mb-231. MCF-10A cells are derived from a 36-year old woman with fibrocystic disease. The expression of SCAP-17 was decreased at least two-fold inbreast carcinoma cells. In addition, SEQ ID NO:42 showed differential expression in human breast epithelial BBMC cells compared to breast carcinoma T-47D cells. The expression of SCAP-17 was decreased at least two-fold in breast carcinoma cells. Therefore, SEQ ID NO:42 is useful in diagnostic assays for cell proliferative disorders, including breast cancer.

In alternative example, SEQ ID NO:43 showed differential expression in lung tissues from patients with cancer compared to matched microscopically normal tissues from the same donors as determined by microarray analysis. The expression of SCAP-18 was decreased at least two-fold in cancerous lung tissue. Therefore, SEQ ID NO:43 is useful in diagnostic assays for cell proliferative disorders, including lung cancer.

XII. Complementary Polynucleotides

Sequences complementary to the SCAP-encoding sequences, or any parts thereof, are used to detect, decrease, or inhibit expression of naturally occurring SCAP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of SCAP. To inhibit transcription, a complementary oligonucleotide is designed from the most unique 5′ sequence and used to prevent promoter binding to the coding sequence. To inhibit translation, a complementary oligonucleotide is designed to prevent nbosomal binding to the SCAP-encoding transcript

XIII. Expression of SCAP

Expression and purification of SCAP is achieved using bacterial or virus-based expression systems. For expression of SCAP in bacteria, cDNA is subcluned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element Recombinant vectors are transformed into suitable bacterial hosts, e.g., BL21(DE3). Antibiotic resistant bacteria express SCAP upon induction with isopropyl beta-D-thiogalactopyranoside (IPTG). Expression of SCAP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding SCAP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. NAtl. Acad. Sci. USA 91:3224-3227; Sandig, V. et al. (1996) Hum. Gene Ther. 7:1937-1945.)

In most expression systems, SCAP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from SCAP at specifically engineered sites. FLAG, an 8-amino acid peptide, enables immunoannnity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified SCAP obtained by these methods can be used directly in the assays shown in Examples XVII and XVIII where applicable.

XIV. Functional Assays

SCAP function is assessed by expressing the sequences encoding SCAP at physiologically elevated levels in mammalian cell culture systems. cDNA is subcloned into a mammalian expression vector containing a strong promoter that drives high levels of cDNA expressionl Vectors of choice include PCMV SPORT plasmid (nvitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Ctometry, Oxford, New York N.Y.

The influence of SCAP on gene expression can be assessed using highly purified populations of cells transfected with sequences encoding SCAP and either CD64 or CD64-GFP. CD64 and CD64-GPP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding SCAP and other genes of interest can be analyzed by northern analysis or microarray techiques.

XV. Production of SCAP Specific Antibodies

SCAP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the SCAP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (See, e.g., Ausubel, 1995, supra, ch. 11.)

Typically, oligopeptides of about 15 residues in length are synthesized using an ABI 431A peptide synthesizer (Applied Biosystems) using FMOC chemistry and coupled to KIH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-SCAP activity by, for example, binding the peptide or SCAP to a substrate, blocking with 1% BSA, reacting with rabbit antisera, washing, and reacting with radio-iodinated goat anti-rabbit IgG.

XVI. Puification of Naturally Occurring SCAP using Specific Antibodies

Naturally occurring or recombinant SCAP is substantially purified by immunoaffinity chromatography using antibodies specific for SCAP. An immunoaffinity column is constructed by covalently coupling anti-SCAP antibody to an activated chromatographic resin, such as CNBr-activated SEPHAROSE (Amersham Biosciences). After the coupling, the resin is blocked and washed according to the manufacturer's instructions.

Media containing SCAP are passed over the immunoaffinity column, and the column is washed under conditions that allow the preferential absorbance of SCAP (e.g., high ionic strength buffers in the presence of detergent). The column is eluted under conditions that disrupt antibody/SCAP binding (e.g., a buffer of pH 2 to pH 3, or a high concentration of a chaotrope, such as urea or thiocyanate ion), and SCAP is collected.

XVII. Identification of Molecules which Interact with SCAP

SCAP, or biologically active fragments thereof, are labeled with ¹²⁵I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled SCAP, washed, and any wells with labeled SCAP complex are assayed. Data obtained using different concentrations of SCAP are used to calculate values for the number, affinity, and association of SCAP with the candidate molecules.

Alternatively, molecules interacting with SCAP are analyzed using the yeast two-hybrid system as described in Fields, S. and O. Song (1989) Nature 340-245-246, or using commercially available kits based on the two-hybrid system, such as the MATCHMAKER system (Clontech).

SCAP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of SCAP Activity

A microtubule motility assay for SCAP measures motor protein activity. In this assay, recombinant SCAP is immobilized onto a glass slide or similar substrate. Taxol-stabilized bovine brain microtubules (commercially available) in a solution containing ATP and cytosolic extract are perfused onto the slide. Movement of microtubules as driven by SCAP motor activity can be visualized and quantified using video-enhanced light microscopy and image analysis techniques. SCAP activity is directly proportional to the frequency and velocity of microtubule movement.

Alternatively, an assay for SCAP measures the formation of protein filaments in vitro. A solution of SCAP at a concentration greater than the “critical concentration” for polymer assembly is applied to carbon-coated grids. Appropriate nucleation sites may be supplied in the solution The grids are negative stained with 0.7% (w/v) aqueous uranyl acetate and examined by electron microscopy. The appearance of filaments of approximately 25 nm (microtubules), 8 nm (actin), or 10 nm (intermediate filaments) is a demonstration of SCAP activity.

In another alternative, SCAP activity is measured by the binding of SCAP to protein filaments. ³⁵S-Met labeled SCAP sample is incubated with the appropriate filament protein (actin, tubulin, or intermediate filament protein) and complexed protein is collected by immunoprecipitation using an antibody against the filament protein. The inaunoprecipitate is then run out on SDS-PAGE and the amount of SCAP bound is measured by autoradiography.

Alternatively, GTP-binding activity of SCAP is determined in an assay that measures the binding of SCAP to [α-³²p]-labeled GTP. Purified SCAP is first blotted onto filters and rinsed in a suitable buffer. The filters are then incubated in buffer containing radiolabeled [α-³²P]-GTP. The filters are washed in buffer to remove unbound GTP and counted in a radioisotope counter. Non-specific binding is determined in an assay that contains a 100-fold excess of unlabeled GTP. The s amount of specific binding is proportional to the activity of SCAP.

Alternatively, SCAP activity may be demonstrated as the ability to interact with its associated LMW GTPase in an in vitro binding assay. The candidate LMW GTPases are expressed as fusion proteins with glutathione S-transferase (GST), and purified by affinity chromatography on glutathione-Sepharose. The LMW GTPases are loaded with GDP by incubating 20 mM Tris buffer, pH 8.0, containing 100 mM NaCl 2 mM EDTA, 5 mM MgCl₂, 0.2 mM DTT, 100 μM AMP-PNP and 10 μM GDP at 30° C. for 20 minutes. SCAP is expressed as a FLAG fusion protein in a baculovirus system. Extracts of these baculovirus cells containing SCAP-FLAG fusion proteins are precleared with GST beads, then incubated with GST-GTPase fusion proteins. The complexes formed are precipitated by glutathione-Sepharose and separated by SDS-polyacrylamide gel electrophoresis. The separated proteins are blotted onto nitrocellulose membranes and probed with commercially available anti-FLAG antibodies. SCAP activity is proportional to the amount of SCAP-FLAG fusion protein detected in the complex.

Alternatively, SCAP activity is measured by its ability to stimulate transcription of a reporter gene (Liu, H. Y. et al. (1997) EMBO J. 16:5289-5298). The assay entails the use of a well characterized reporter gene construct, LexAₒₚ-LacZ, that consists of LexA DNA transcriptional control elements (LexAₒₚ) fused to sequences encoding the E. coli LacZ enzyme. The methods for constructing and expressing fusion genes, introducing them into cells, and measuring LacZ enzyme activity, are well known to those skilled in the art. Sequences encoding SCAP are cloned into a plasmid that directs the synthesis of a fusion protein, LexA-SCAP, consisting of SCAP and a DNA binding domain derived from the LexA transcription factor. The resulting plasmid, encoding a lexA-SCAP fusion protein, is introduced into yeast cells along with a plasmid containing the LexAₒₚ-LacZ reporter gene. The amount of LacZ enzyme activity associated with LexA-SCAP transfected cells, relative to control cells, is proportional to the amount of transcription stimulated by the SCAP.

Alternatively, SCAP activity is measured by its ability to bind zinc. A 5-10 micromolar sample solution in 2.5 mM ammonium acetate solution at pH 7.4 is combined with 0.05 M zinc sulfate solution (Aldrich, Milwaukee Wis.) in the presence of 100 micromolar dithiothreitol with 10% methanol added. The sample and zinc sulfate solutions are allowed to incubate for 20 minutes. The reaction solution is passed through a Vydac column with approximately 300 Angstrom bore size and 5 micromolar particle size to isolate zinc-sample complex from the solution, and into a mass spectrometer (PE Sciex, ontario, Canada). Zinc bound to sample is quantified using the fuctional atomic mass of 63.5 Da bserved by Whittal, R. M. et al. ((2000) Biochemistry 39:8406-8417).

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention It is intended that the scope of the invention be defined by the following claims and their equivalents.

TABLE 1

| | Polypep- | | | Incyte |
|---|---|---|---|---|
| Incyte | tide SEQ | Incyte | Polynucleotide | Polynucleotide |
| Project ID | ID NO: | Polypeptide ID | SEQ ID NO: | ID |
| 2780338 | 1 | 2780338CD1 | 26 | 2780338CB1 |
| 2317440 | 2 | 2317440CD1 | 27 | 2317440CB1 |
| 3013470 | 3 | 3013470CD1 | 28 | 3013470CB1 |
| 1738823 | 4 | 1738823CD1 | 29 | 1738823CB1 |
| 4184551 | 5 | 4184551CD1 | 30 | 4184551CB1 |
| 70042484 | 6 | 70042484CD1 | 31 | 70042484CB1 |
| 3236274 | 7 | 3236274CD1 | 32 | 3236274CB1 |
| 7179725 | 8 | 7179725CD1 | 33 | 7179725CB1 |
| 1966217 | 9 | 1966217CD1 | 34 | 1966217CB1 |
| 1598186 | 10 | 1598186CD1 | 35 | 1598186CB1 |
| 7493044 | 11 | 7493044CD1 | 36 | 7493044CB1 |
| 7925017 | 12 | 7925017CD1 | 37 | 7925017CB1 |
| 6758789 | 13 | 6758789CD1 | 38 | 6758789CB1 |
| 7488249 | 14 | 7488249CD1 | 39 | 7488249CB1 |
| 5046311 | 15 | 5046311CD1 | 40 | 5046311CB1 |
| 931056 | 16 | 931056CD1 | 41 | 931056CB1 |
| 2578937 | 17 | 2578937CD1 | 42 | 2578937CB1 |
| 489786 | 18 | 489786CD1 | 43 | 489786CB1 |
| 2240034 | 19 | 2240034CD1 | 44 | 2240034CB1 |
| 3438037 | 20 | 3438037CD1 | 45 | 3438037CB1 |
| 6578021 | 21 | 6578021CD1 | 46 | 6578021CB1 |
| 8013295 | 22 | 8013295CD1 | 47 | 8013295CB1 |
| 5001859 | 23 | 5001859CD1 | 48 | 5001859CB1 |
| 7506133 | 24 | 7506133CD1 | 49 | 7506133CB1 |
| 5301066 | 25 | 5301066CD1 | 50 | 5301066CB1 |

TABLE 2

| Polypep- | | | | |
|---|---|---|---|---|
| tide | | GenBank ID NO: | | |
| SEQ | Incyte | or PROTEOME | Probability | |
| ID NO: | Polypeptide ID | ID NO: | Score | Annotation |
| 1 | 2780338CD1 | g1845265 | 5.7E−76 | [Homo sapiens] ankyrin (Gallagher, P. G. et al. (1997) J. Biol. Chem. 272 (31), |
| | | | | 19220-19228) |
| 2 | 2317440CD1 | g6739602 | 0.0E+00 | [Homo sapiens] talin (Ben-Yosef, T. et al. (1999) Genomics 62 (2), 316-319) |
| 3 | 3013470CD1 | g6934244 | 3.7E−50 | [Homo sapiens] tropomodulin 3 (Cox, P. R. et al. (2000) Genomics 63 (1), 97-107) |
| 4 | 1738823CD1 | g2961133 | 4.5E−34 | [Drosophila melanogaster] nuclear fallout (Rothwell, W. F. et al. (1998) |
| | | | | Development 125 (7), 1295-1303) |
| 4 | 1738823CD1 | g15991574 | 9.0E−90 | [Homo sapiens] eferin (Prekeris, R. et al. (2001) J. Biol. Chem. 276 (42), 38966-38970) |
| 5 | 4184551CD1 | g4778 | 1.4E−29 | [Saccharomyces cerevisiae] Uso1 protein (Nakajima, H. et al. (1991) J. Cell Biol. |
| | | | | 113 (2), 245-260) |
| 6 | 70042484CD1 | g2853592 | 0.0E+00 | [Rattus norvegicus] neurabin II (Satoh, A. et al. (1998) J. Biol. Chem. 273 (6), |
| | | | | 3470-3475) |
| 7 | 3236274CD1 | g6118086 | 7.7E−181 | [Homo sapiens] adaptor protein APPL (Mitsuuchi, Y. et al. (1999) Oncogene 18 |
| | | | | (35), 4891-4898) |
| 8 | 7179725CD1 | g2769587 | 1.2E−205 | [Mus musculus] STOP protein (Denarier, E. et al. (1998) Biochem. Biophys. Res. |
| | | | | Commun. 243 (3), 791-796) |
| 9 | 1966217CD1 | g3342246 | 0.0E+00 | [Rattus norvegicus] actin-filament binding protein Frabin (Obaishi, H. et al. (1998) |
| | | | | J. Biol. Chem. 273 (30), 18697-18700) |
| 10 | 1598186CD1 | g2773060 | 7.3E−34 | [Homo sapiens] actinin-associated LIM protein (Pietu, G. et al. (1996) Genome |
| | | | | Res. 6 (6), 492-503) |
| 10 | 1598186CD1 | g10445215 | 1.0E−151 | PDZ-LIM protein mystique [Homo sapiens] |
| 11 | 7493044CD1 | g34037 | 3.7E−158 | [Homo sapiens] cytokeratin 18 (Oshima, R. G. et al. (1986) Differentiation 33 (1), |
| | | | | 61-68) |
| 12 | 7925017CD1 | g7329078 | 6.3E−27 | [Homo sapiens] intersectin 2 short isoform (Pucharcos, C. et al. (2000) FEBS Lett. |
| | | | | 478 (1-2), 43-51) |
| 13 | 6758789CD1 | g2337952 | 4.6E−172 | [Homo sapiens] actin-binding double-zinc-finger protein (Roof, D. J. et al. (1997) |
| | | | | J. Cell Biol. 138 (3), 575-588) |
| 14 | 7488249CD1 | g3378196 | 1.5E−88 | [Homo sapiens] nebulette (Millevoi, S. et al. (1998) J. Mol. Biol. 282 (1), 111-123) |
| 15 | 5046311CD1 | g4761644 | 1.6E−89 | [Rattus norvegicus] myomegalin (Verde, I. Et al. (2001) J. Biol. Chem. 276 (14), |
| | | | | 11189-11198) |
| 16 | 931056CD1 | g10334699 | 1.3E−13 | [Homo sapiens] plakoglobin (Whittock, N. V. et al. (2000) Exp. Dermatol. 9 (5), |
| | | | | 323-326) |
| 17 | 2578937CD1 | g2337952 | 3.4E−147 | [Homo sapiens] actin-binding double-zinc-finger protein (Roof, D. J. et al. (1997) |
| | | | | J. Cell Biol. 138 (3), 575-588) |
| 18 | 489786CD1 | g178646 | 6.6E−81 | [Homo sapiens] ankyrin (Lambert, S. et al. (1990) Proc. Nati. Acad. Sci. U.S.A. 87 |
| | | | | (5), 1730-1734) |
| 19 | 2240034CD1 | g4406218 | 4.6E−219 | [Homo sapiens] echinoderm microtubule-associated protein-like EMAP2 |
| | | | | (Lepley, D. M. et al. (1999) Gene 237 (2), 343-349) |
| 20 | 3438037CD1 | g998899 | 2.5E−99 | [Mus sp.] scleraxis = basic helix-loop-helix transcription factor [mice, embryos, |
| | | | | Peptide, 207 aa] (Cserjesi, P. et al. (1995) Development 121 (4), 1099-1110) |
| 21 | 6578021CD1 | g4406218 | 3.6E−204 | [Homo sapiens] echinoderm microtubule-associated protein-like EMAP2 |
| | | | | (Lepley, D. M. et al. (1999) Gene 237 (2), 343-349) |
| 22 | 8013295CD1 | g710549 | 0.0E+00 | [Mus musculus] ankyrin 3 (Peters, L. L. et al. (1995) J. Cell Biol. 130 (2), 313-330) |
| 23 | 5001859CD1 | g10441465 | 2.4E−118 | [Homo sapiens] actin filament associated protein |
| 24 | 7506133CD1 | g6118086 | 1.3E−166 | [Homo sapiens] adaptor protein APPL (Mitsuuchi, Y. et al. (1999) Oncogene 18 |
| | | | | (35), 4891-4898) |
| 24 | 7506133CD1 | 429068\|APPL | 1.2E−167 | [Homo sapiens][Protein kinase; Transferase; Small molecule-binding |
| | | | | protein][Cytoplasmic] Adaptor protein that contains a pleckstrin homology (PH), |
| | | | | a phosphotyrosine binding (PTB) domain, and a leucine zipper motif, binds to |
| | | | | AKT2 and PI3 kinase catalytic subunit p110alpha (PIK3CA) |
| 24 | 7506133CD1 | 425420\|CENTB2 | 4.3E−21 | [Homo sapiens] Putative member of the GTPase activating protein for Arf family, |
| | | | | contains a plexin repeat and two ankyrin (Ank) repeats, has low similarity to |
| | | | | centaurin beta 1B (C elegans cnt-1A), which is involved in phosphoinositide |
| | | | | signaling |
| 24 | 7506133CD1 | 570228\|GRAF | 2.5E−12 | [Homo sapiens][GTPase activating protein;Activator][Nuclear;Cytoplasmic] |
| | | | | Protein with high similarity to OPHN1, which is a putative Rho -GTPase- |
| | | | | activating protein associated with nonspecific X-linked mental retardation (MRX); |
| | | | | contains a RhoGAP domain, a pleckstrin homology (PH) domain, and an Src |
| | | | | homology 3 (SH3) domain |
| 25 | 5301066CD1 | g2337952 | 1.7E−46 | [Homo sapiens] actin-binding double-zinc-finger protein (Roof, D. J. et al. (1997) |
| | | | | J. Cell Biol. 138 (3), 575-588) |

TABLE 3

| | | Amino | | | | |
|---|---|---|---|---|---|---|
| SEQ | Incyte | Acid | Potential | Potential | | Analytical |
| ID | Polypeptide | Resi- | Phosphorylation | Glycosylation | | Methods |
| NO: | ID | dues | Sites | Sites | Signature Sequences, Domains and Motifs | and Databases |
| 1 | 2780338CD1 | 1250 | S14 S55 S60 S74 | N20 N198 N426 | Ank repeat: E634-D666, N601-S633, D431-A463, | HMMER_PFAM |
| | | | S231 S330 S348 | N723 N957 | D700-Y732, E832-N864, D530-K567; D766-A798, | |
| | | | S725 S796 S946 | N1002 N1010 | F898-S930, K313-S364, D568-N600, Q865-Q897, | |
| | | | S970 S984 S991 | N1032 N1111 | E799-N831, E497-V529, H398-Q430, D733-A765, | |
| | | | S1004 S1007 S1012 | | A667-N699, N365-A397, D464-N496 | |
| | | | S1034 S1072 S1082 | | | |
| | | | S1085 S1097 S1221 | | | |
| | | | S1226 T22 T186 | | | |
| | | | T202 T243 T269 | | | |
| | | | T351 T428 T653 | | | |
| | | | T781 T953 T1123 | | | |
| | | | T1205 T1248 Y732 | | | |
| | | | | | Transmembrane domain: D599-W624 N-terminus is | TMAP |
| | | | | | non-cytosolic | |
| | | | | | Domain present in ZO-1 a PF00791: G420-W474, | BLIMPS_PFAM |
| | | | | | L540-D594, Q897-V949 | |
| | | | | | Ank repeat proteins. PF00023: L573-L588, G668-F677 | BLIMPS_PFAM |
| | | | | | T28D6.4 Ankyrin-repeat PROTEIN PD067835: | BLAST_{—} |
| | | | | | I727-V806; (P-2.9e−10) | PRODOM |
| | | | | | Leucine zipper pattern: L271-L292 | MOTIFS |
| 2 | 2317440CD1 | 2542 | S5 S54 S98 S263 | N190 N227 N440 | FERM domain (Band 4.1 family): I123-L308 | HMMER_PFAM |
| | | | S293 S307 S365 | N503 N562 N586 | | |
| | | | S408 S428 S461 | N661 N1252 | | |
| | | | S517 S542 S548 | N1309 N1536 | | |
| | | | S607 S793 S818 | N1573 N2125 | | |
| | | | S845 S856 S944 | N2207 N2413 | | |
| | | | S957 S1005 S1032 | | | |
| | | | S1040 S1143 S1156 | | | |
| | | | S1217 S1221 S1222 | | | |
| | | | S1233 S1254 S1273 | | | |
| | | | S1314 S1390 | | | |
| | | | S1397 S1515 S1530 | | I/L WEQ domain: F2342-L2533, L671-E765, | HMMER_{—} |
| | | | S1579 S1604 S1645 | | H1526-V1542, A849-Q891, V922-D981 | PFAM |
| | | | S1652 S1738 S1837 | | | |
| | | | S2040 S2171 S2172 | | | |
| | | | S2176 S2196 S2209 | | | |
| | | | S2274 S2407 S2428 | | | |
| | | | S2468 | | | |
| | | | T109 T124 T135 | | Transmembrane domain: D664-N691, Q1735-I1754, | TMAP |
| | | | T146 T169 T181 | | Q1977-F1999 N-terminus is non-cytosolic | |
| | | | T358 T386 T421 | | | |
| | | | T433 T442 T564 | | | |
| | | | T632 T762 T870 | | | |
| | | | T1012 T1061 | | | |
| | | | T1096 T1296 | | | |
| | | | T1374 T1544 | | | |
| | | | T1607 T1618 | | | |
| | | | T1641 T1691 | | | |
| | | | T1762 T1769 | | | |
| | | | T1814 T1843 | | | |
| | | | T1850 T1868 | | | |
| | | | T1878 T1947 | | | |
| | | | T1959 T2012 | | | |
| | | | T2023 T2132 | | | |
| | | | T2187 Y83 Y315 | | | |
| | | | Y1614 Y2113 | | | |
| | | | | | Band 4.1 family domain signatures and profile: | PROFILESCAN |
| | | | | | S179-D224 | |
| | | | | | TALIN INTEGRIN STRUCTURAL PROTEIN | BLAST_{—} |
| | | | | | CYTOSKELETON PHOSPHORYLATION | PRODOM |
| | | | | | PD018644: Q951-L2252, C289-M672, V692-Q933 | |
| | | | | | TALIN STRUCTURAL PROTEIN | BLAST_{—} |
| | | | | | CYTOSKELETON INTEGRIN | PRODOM |
| | | | | | PHOSPHORYLATION FILOPODIN HOMOLOG | |
| | | | | | ACTINBINDING PD024383: M1-I123 | |
| | | | | | PROTEIN CYTOSKELETON TALIN | BLAST_{—} |
| | | | | | STRUCTURAL ACTINBINDING INTEGRIN | PRODOM |
| | | | | | PHOSPHORYLATION FILOPODIN HOMOLOG | |
| | | | | | ZK370.3 PD011820: E2300-Y2530 | |
| | | | | | TALIN STRUCTURAL PROTEIN | BLAST_{—} |
| | | | | | CYTOSKELETON PHOSPHORYLATION | PRODOM |
| | | | | | PD149438: E2253-G2312 | |
| 3 | 3013470CD1 | 560 | S8 S28 S37 S70 | N26 N237 N309 | WH2 motif involved in actin reorganization: P534-V553, | HMMER_PFAM |
| | | | S81 S134 S209 | N337 N367 | V553, e value = 0.83 | |
| | | | S214 S231 S244 | | | |
| | | | S463 S473 S484 | | | |
| | | | T58 T101 T143 | | | |
| | | | T169 T192 T239 | | | |
| | | | T323 T339 T480 | | | |
| | | | T517 T533 Y76 | | | |
| | | | | | TROPOMODULIN SIMILAR AUTOANTIGEN D1 | BLAST_{—} |
| | | | | | THYROIDASSOCIATED OPHTHALMOPATHY | PRODOM |
| | | | | | ANTIGEN REPEAT TRANSMEMBRANE | |
| | | | | | SANPODO PD007741: R82-K396 | |
| | | | | | PROTEIN COILED COIL CHAIN MYOSIN | BLAST_{—} |
| | | | | | REPEAT HEAVY ATPBINDING FILAMENT | PRODOM |
| | | | | | HEPTAD PD000002: D9-K215 | |
| | | | | | do TROPOMODULIN; DM08203\|P29536\|239-465: | BLAST_DOMO |
| | | | | | K197-K415 \|P28289\|27-358: N237-R401, L32-T93 | |
| 4 | 1738823CD1 | 470 | S55 S69 S137 S157 | N150 N155 N189 | NUCLEAR FALLOUT KIAA0665 PROTEIN | BLAST_{—} |
| | | | S178 S278 S291 | | GLUTAMICRICH PD031147: R371-K469 | PRODOM |
| | | | S298 S404 S429 | | | |
| | | | S464 T18 T90 | | | |
| | | | T174 T185 T193 | | | |
| | | | T224 T250 T280 | | | |
| | | | Y308 | | | |
| | | | | | PROTEIN COILED COIL CHAIN MYOSIN | BLAST_{—} |
| | | | | | REPEAT HEAVY ATPBINDING FILAMENT | PRODOM |
| | | | | | HEPTAD PD000002: E175-E385 | |
| | | | | | TRICHOHYALIN DM03839\|P37709\|632-1103: | BLAST_DOMO |
| | | | | | E159-K453 | |
| | | | | | Leucine zipper pattern: L255-L276, L262-L283, | MOTIFS |
| | | | | | L375-L396, L382-L403 | |
| 5 | 4184551CD1 | 898 | S2 S12 S31 S35 | N33 N40 N109 | PROTEIN COILED COIL CHAIN MYOSIN | BLAST_{—} |
| | | | S36 S130 S139 | N867 | REPEAT HEAVY ATPBINDING FILAMENT | PRODOM |
| | | | S157 S228 S256 | | HEPTAD PD000002: L332-E565 | |
| | | | S405 S581 S586 | | | |
| | | | S611 S617 S625 | | | |
| | | | S690 S734 S735 | | | |
| | | | S825 T10 T49 T85 | | | |
| | | | T126 T135 T146 | | | |
| | | | T241 T243 T250 | | | |
| | | | T278 T347 T371 | | | |
| | | | T417 T440 T459 | | | |
| | | | T490 T500 T515 | | | |
| | | | T592 T596 T671 | | | |
| | | | T682 T797 T869 | | | |
| | | | T879 T895 Y141 | | | |
| | | | Y522 | | | |
| | | | | | TRICHOHYALIN DM03839\|P37709\|632-1103: | BLAST_DOMO |
| | | | | | E229-K704 | |
| | | | | | Do; MYOSIN-LIKE PROTEIN | BLAST_DOMO |
| | | | | | DM07884\|Q02455\|35-1728: | |
| | | | | | K82-T898; \|P40457\|16-1657: F194-K897 | |
| | | | | | do SCII; ASSEMBLY; SCAFFOLD; | BLAST_DOMO |
| | | | | | CHROMOSOMAL; DM03788\|A54817\|331-1178: | |
| | | | | | I438-I800 | |
| | | | | | Leucine zipper pattern: L588-L609, L870-L891 | MOTIFS |
| 6 | 70042484CD1 | 817 | S21 S87 S100 S116 | N190 N331 | PDZ domain (Also known as DHR or GLGF).: | HMMER_PFAM |
| | | | S118 S122 S245 | P496-E583 | | |
| | | | S309 S382 S388 | | | |
| | | | S408 S438 S446 | | | |
| | | | S463 S658 S712 | | | |
| | | | S756 S802 T147 | | | |
| | | | T530 T628 T681 | | | |
| | | | T777 T810 Y613 | | | |
| | | | | | SPINOPHILIN NEURABIN PD042675: M1-S388 | BLAST_{—} |
| | | | | | | PRODOM |
| | | | | | SPINOPHILIN PD031166: R584-F671 | BLAST_{—} |
| | | | | | | PRODOM |
| | | | | | NEURABIN KS5 PROTEIN PD075355: R584-E663 | BLAST_{—} |
| | | | | | | PRODOM |
| | | | | | SPINOPHILIN NEURABIN SIMILAR PROTEIN | BLAST_{—} |
| | | | | | | PRODOM |
| | | | | | TYROSINE PHOSPHATASES PD022951: G436-F495 | |
| | | | | | GLGF DOMAIN DM00224\|A56158\|832-924: | BLAST_DOMO |
| | | | | | L485-T572 GLGF DOMAIN DM00224\|P55196\| | BLAST_DOMO |
| | | | | | 980-1073: E493-V576 | |
| | | | | | Leucine zipper pattern: L485-L506 | MOTIFS |
| | | | | | Binding-protein-dependent transport systems inner | MOTIFS |
| | | | | | membrane comp. sign: M64-R92 | |
| 7 | 3236274CD1 | 664 | S24 S219 S228 | N288 N327 N372 | Pleckstrin Homology (PH) domain: I278-R375 | HMMER_PFAM |
| | | | S250 S256 S359 | N391 | | |
| | | | S420 S436 S468 | | | |
| | | | S481 S508 S543 | | | |
| | | | S585 S598 S600 | | | |
| | | | T32 T128 T295 | | | |
| | | | T477 T586 | | | |
| | | | | | Phosphotyrosine interaction domain (PTB/PID): | HMMER_PFAM |
| | | | | | F494-S585 | |
| 8 | 7179725CD1 | 780 | S61 S136 S231 | | signal_cleavage: M1-A36 | SPSCAN |
| | | | S300 S381 S388 | | | |
| | | | S390 S412 S448 | | | |
| | | | S558 S679 T238 | | | |
| | | | T271 T348 T394 | | | |
| | | | T418 T419 T457 | | | |
| | | | T510 Y336 | | | |
| | | | | | PROTEIN STOP ESTOP MICROTUBULE- | BLAST_{—} |
| | | | | | ASSOCIATED NEURONAL STOP PUTATIVE | PRODOM |
| | | | | | MICROTUBULE ASSOCIATED PD017800: M1-R161 | |
| | | | | | PROTEIN MICROTUBULE-ASSOCIATED STOP | BLAST_{—} |
| | | | | | NEURONAL STOP FSTOP ESTOP PUTATIVE | PRODOM |
| | | | | | MICROTUBULE ASSOCIATED PD014085: E94-K373 | |
| | | | | | PROTEIN STOP MICROTUBULE-ASSOCIATED | BLAST_{—} |
| | | | | | NEURONAL STOP P53 BINDING 53BP2 BCL2 | PRODOM |
| | | | | | BINDING BBP SH3 DOMAIN PD017969: S440-P771 | |
| | | | | | PROTEIN STOP MICROTUBULE-ASSOCIATED | BLAST_{—} |
| | | | | | NEURONAL STOP ESTOP PUTATIVE | PRODOM |
| | | | | | MICROTUBULE- ASSOCIATED PD017799: | |
| | | | | | V374-E439 do NEUROFILAMENT; TRIPLET; | BLAST_DOMO |
| | | | | | DM04498\|P12036\|434-1019: Q199-P757 | |
| | | | | | Cell attachment sequence: R168-D170 | MOTIFS |
| 9 | 1966217CD1 | 766 | S25 S43 S155 S186 | N35 N77 N150 | FYVE zinc finger: P554-G620 | HMMER_PFAM |
| | | | S325 S438 S493 | N256 N315 N436 | | |
| | | | S505 S545 S708 | | | |
| | | | S764 T79 T217 | | | |
| | | | T282 T317 T466 | | | |
| | | | T481 T529 T576 | | | |
| | | | T622 T753 | | | |
| | | | | | PH domain: L423-D521, S644-T740 | HMMER_PFAM |
| | | | | | RhoGEF domain: I210-S392 | HMMER_PFAM |
| | | | | | ACTIN FILAMENT BINDING PROTEIN FRABIN | BLAST_{—} |
| | | | | | PD179644: M1-A211 | PRODOM |
| | | | | | PROTEIN FACIOGENITAL DYSPLASIA | BLAST_{—} |
| | | | | | PUTATIVE RHO/RAC GUANINE NUCLEOTIDE | PRODOM |
| | | | | | EXCHANGE FACTOR GEF PD038082: S392-A553 | |
| | | | | | PD035184: T622-T743 | |
| | | | | | PROTEIN FACTOR GUANINE NUCLEOTIDE | BLAST_{—} |
| | | | | | RELEASING NUCLEOTIDE GUANINE | PRODOM |
| | | | | | EXCHANGE PROTOONCOGENE BINDING SH3 | |
| | | | | | PD000777: I210-A393 | |
| | | | | | BCR PROTEIN DM08397\|A49307\|26-564: T144-L452 | BLAST_DOMO |
| | | | | | GUANINE-NUCLEOTIDE DISSOCIATION | BLAST_DOMO |
| | | | | | STIMULATORS CDC24 FAMILY | |
| | | | | | DM08581\|P11433\|105-684: D194-A506 | |
| | | | | | do VAV; KINASE; ZINC; SH2; | BLAST_DOMO |
| | | | | | DM08580\|P52735\|1-491: E198-K457 | |
| 10 | 1598186CD1 | 420 | S13 S149 S161 | | PDZ domain (Also known as DHR or GLGF).: | HMMER_PFAM |
| | | | S171 S183 S227 | | L57-S137 | |
| | | | S256 S295 T8 T29 | | | |
| | | | T49 T145 T164 | | | |
| | | | T366 T412 Y359 | | | |
| | | | | | PROTEIN DOMAIN PROTEASE PHOSPHATASE | BLAST_{—} |
| | | | | | SH3 REPEAT PDZ TYROSINE PRECURSOR | PRODOM |
| | | | | | HYDROLASE PD000073: L57-P129 | |
| | | | | | do ENIGMA; LIM; RIL; DM03985\|P50479\|1-242: | BLAST_DOMO |
| | | | | | M55-R233 DM03985\|A55050\|1-270: V59-L268 | |
| | | | | | DM03985\|P52944\|1-247: G63-R297 | |
| 11 | 7493044CD1 | 417 | S27 S84 S91 S305 | N189 | Intermediate filament protein: N77-N384 | HMMER_PFAM |
| | | | S316 S413 T128 | | | |
| | | | T345 Y265 Y324 | | | |
| | | | Y373 | | | |
| | | | | | Intermediate filaments p BL00226: N77-S91, | BLIMPS_{—} |
| | | | | | V174-A221, D239-Q269, L338-N384 | BLOCKS |
| | | | | | Intermediate filaments signature: Q350-T403 | PROFILESCAN |
| | | | | | FILAMENT INTERMEDIATE REPEAT HEPTAD | BLAST_{—} |
| | | | | | PATTERN COILED COIL KERATIN: PD000194: | PRODOM |
| | | | | | I75-N384 | |
| | | | | | KERATIN TYPE I CYTOSKELETAL | BLAST_{—} |
| | | | | | CYTOKERATIN INTERMEDIATE FILAMENT | PRODOM |
| | | | | | COILED COIL HEPTAD: PD028287: T8-Q76 | |
| | | | | | INTERMEDIATE FILAMENTS | BLAST_DOMO |
| | | | | | DM00061\|P05783\|52-417: S51-V412 | |
| | | | | | DM00061\|P02533\|69-452: G38-V412 | |
| | | | | | DM00061\|Q04695\|28-421: A37-S413 | |
| | | | | | DM00061\|JC4313\|68-455: Y34-T403 | |
| | | | | | Intermediate filaments signature: I370-E378 | MOTIFS |
| 12 | 7925017CD1 | 567 | S45 S70 S74 S135 | N142 N292 | SH3 domain: E441-C495, R510-L564, S351-V405, | HMMER_PFAM |
| | | | S144 S146 S155 | | P275-I329 | |
| | | | S212 S368 S370 | | | |
| | | | S417 S458 S527 | | | |
| | | | S539 T31 T35 | | | |
| | | | T132 T238 T250 | | | |
| | | | T294 T424 T453 | | | |
| | | | T535 Y229 | | | |
| | | | | | SH3 domain signature: PR00452: D455-R470, | BLIMPS_{—} |
| | | | | | D472-Q481 | PRINTS |
| | | | | | Neutrophil cytosol factor 2 signature: | BLIMPS_{—} |
| | | | | | PR00499: D532-E548, E366-W385, G392-S411 | PRINTS |
| | | | | | KRYN: PD136084: L9-H276 | BLAST_{—} |
| | | | | | | PRODOM |
| | | | | | SRC HOMOLOGY 3 (SH3) DOMAIN: | BLAST_DOMO |
| | | | | | DM00025\|P53281\|54-107: L280-K327 | |
| | | | | | Cell attachment sequence: R215-D217, R461-D463 | MOTIFS |
| 13 | 6758789CD1 | 648 | S54 S382 S388 | | signal_cleavage: M1-S47 | SPSCAN |
| | | | S398 S468 S521 | | | |
| | | | S564 S615 S626 | | | |
| | | | T262 T320 T421 | | | |
| | | | T566 T594 T608 | | | |
| | | | Y352 | | | |
| | | | | | LIM domain: C195-G251, C125-C176, C66-G122, | HMMER_PFAM |
| | | | | | C254-L295 | |
| | | | | | Villin headpiece domain: H613-F648 | HMMER_PFAM |
| | | | | | LIM domain signature and profile lim_domain: | PROFILESCAN |
| | | | | | R107-K249, S44-Y117 | |
| | | | | | PROTEIN DEMATIN ACTIN BINDING | BLAST_{—} |
| | | | | | ERYTHROCYTE MEMBRANE BAND CAPPING | PRODOM |
| | | | | | REPEAT PHOSPHORYLATION A PD017047: | |
| | | | | | E312-D531 | |
| | | | | | PROTEIN ACTIN BINDING CAPPING REPEAT | BLAST_{—} |
| | | | | | VILLIN CALCIUM PUTATIVE SUPER VILLIN | PRODOM |
| | | | | | DEMATIN ADVILLIN PD003485: K584-F648 | |
| | | | | | VILLIN HEADPIECE DM04001\|Q08495\|252-382: | BLAST_DOMO |
| | | | | | P553-F648 | |
| | | | | | LIM domain signature C66-L99 C125-F158 C195-L299 | MOTIFS |
| | | | | | C254-F287 | |
| 14 | 7488249CD1 | 270 | S202 S222 S230 | | LIM domain: C5-F62 | HMMER_PFAM |
| | | | T15 T64 T143 | | | |
| | | | T150 T214 T255 | | | |
| | | | Y52 | | | |
| | | | | | Nebulin repeat: D103-G131, D67-G95, D139-E167 | HMMER_PFAM |
| | | | | | SH3 domain: R213-V269 | HMMER_PFAM |
| | | | | | LIM domain proteins BL00478: P3-K10, W25-L39 | BLIMPS_{—} |
| | | | | | | BLOCKS |
| | | | | | Src homology 3 (SH3) domain proteins profile | BLIMPS_{—} |
| | | | | | BL50002: A217-D235, T255-F268 | BLOCKS |
| | | | | | LIM domain signature and profile lim_domain.prf: | PROFILESCAN |
| | | | | | P3-Y57 | |
| | | | | | SH3 domain signature PR00452: R213-A223, | BLIMPS_{—} |
| | | | | | D227-P242 D244-Q253, R257-V269 | PRINTS |
| | | | | | NEBULETTE NEBULIN PD097585: F134-P172, | BLAST_{—} |
| | | | | | R171-S209, F98-Y122 | PRODOM |
| | | | | | NEBULIN LIM PROTEIN MOTIF METAL | BLAST_{—} |
| | | | | | BINDING ZINC NEBULETTE NEBULIN | PRODOM |
| | | | | | RELATED ANCHORING NRAP PD001795: | |
| | | | | | P58-K159, Q60-K159 | |
| | | | | | LIM METAL-BINDING REPEAT | BLAST_DOMO |
| | | | | | DM00055\|P34417\|1-62: M1-K59 | |
| | | | | | SRC HOMOLOGY 3 (SH3) DOMAIN | BLAST_DOMO |
| | | | | | DM00025\|S55024\|6611-6666: R213-E267 | |
| | | | | | SRC HOMOLOGY 3 (SH3) DOMAIN | BLAST_DOMO |
| | | | | | DM00025\|P34416\|141-196: R216-E267 | |
| | | | | | LIM domain signature C5-L39 | MOTIFS |
| 15 | 5046311CD1 | 1893 | S25 S112 S133 | N46 N253 N387 | PROTEIN COILED COIL CHAIN MYOSIN | BLAST_{—} |
| | | | S229 S255 S287 | N1104 N1107 | REPEAT HEAVY ATP BINDING FILAMENT | PRODOM |
| | | | S313 S339 S374 | N1162 N1340 | HEPTAD PD000002: K270-L501, E268-Q477, | |
| | | | S400 S430 S468 | N1378 N1690 | Q282-Q506 | |
| | | | S547 S548 S570 | | | |
| | | | S649 S658 S706 | | | |
| | | | S757 S791 S1020 | | | |
| | | | S1064 S1083 S1089 | | | |
| | | | S1164 S1288 S1360 | | | |
| | | | S1365 S1412 S1440 | | | |
| | | | S1485 S1500 S1513 | | | |
| | | | S1529 S1548 S1556 | | MYOSIN-LIKE PROTEIN MLP1 | BLAST_DOMO |
| | | | S1643 S1680 S1688 | | DM07884\|Q02455\|35-1728: R56-E1492, V1198-K1679, | |
| | | | S1804 S1849 T102 | | R1197-Q1859 | |
| | | | T161 T185 T187 | | | |
| | | | T308 T405 T435 | | | |
| | | | T525 T607 T677 | | | |
| | | | T712 T810 T823 | | | |
| | | | T826 T878 T929 | | | |
| | | | T1105 T1121 | | | |
| | | | T1328 T1380 | | | |
| | | | T1404 T1677 | | | |
| | | | T1762 T1785 | | | |
| | | | T1789 T1842 | | | |
| | | | T1863 Y461 Y1211 | | | |
| | | | Y1343 Y1794 | | | |
| | | | | | TRICHOHYALIN DM03839\|P37709\|632-1103: | BLAST_DOMO |
| | | | | | E109-D562, E224-D659, E49-K489 | |
| | | | | | DM03839\|P22793\|921-1475: E141-K656, P95-K656 | |
| | | | | | ATP/GTP-binding site motif A (P-loop) G816-T823 | MOTIFS |
| | | | | | Leucine zipper pattern L653-L674 L660-L681 | MOTIFS |
| | | | | | L788-L809 L1533-L1554 L1540-L1561 | |
| 16 | 931056CD1 | 869 | S24 S36 S96 S160 | N59 N86 N135 | Armadillo/beta-catenin-like repeat: D305-E345, | HMMER_PFAM |
| | | | S170 S281 S327 | N285 N382 N530 | N346-T385, N387-M427, D469-G509, | |
| | | | S367 S408 S409 | N551 N828 | E139-Q179, G56-S96, D180-N220, D221-E262, | |
| | | | S491 S553 S614 | | D263-Y304, N97-A138 | |
| | | | S615 S633 S637 | | | |
| | | | S638 S652 S662 | | | |
| | | | S708 S716 S830 | | | |
| | | | T62 T77 T141 | | | |
| | | | T287 T466 T513 | | | |
| | | | T534 Y50 Y175 | | | |
| | | | Y544 Y644 Y866 | | | |
| | | | | | do CATENIN; BETA; PLAKOGLOBIN; | BLAST_DOMO |
| | | | | | SEGMENT; DM01792\|P35223\|15-766: A68-T466, | |
| | | | | | V455-E511 DM01792\|P14923\|1-719: A68-V529 | |
| | | | | | DM01792\|P18824\|21-764: A68-T466 | |
| | | | | | DM01792\|P26233\|1-741: A68-T464, R432-M505 | |
| 17 | 2578937CD1 | 572 | S84 S124 S139 | N2 | signal_cleavage: M1-A57 | SPSCAN |
| | | | S218 S277 S310 | | | |
| | | | S372 S382 S392 | | | |
| | | | S393 S426 S439 | | | |
| | | | S456 S539 S552 | | | |
| | | | T29 T80 T244 | | | |
| | | | T252 T350 T357 | | | |
| | | | T518 T532 T550 | | | |
| | | | | | LIM domain: C151-G207, C82-A138, C23-G79, | HMMER_PFAM |
| | | | | | C210-L251 | |
| | | | | | Villin headpiece domain: H537-F572 | HMMER_PFAM |
| | | | | | LIM domain signature and profile: K64-Q205, | PROFILESCAN |
| | | | | | F62-A264 | |
| | | | | | PROTEIN ACTIN BINDING CAPPING REPEAT | BLAST_{—} |
| | | | | | VILLIN CALCIUM PUTATIVE SUPER VILLIN | PRODOM |
| | | | | | DEMATIN ADVILLIN PD003485: R485-F572 | |
| | | | | | VILLIN HEADPIECE DM04001\|Q08495\|252-382: | BLAST_DOMO |
| | | | | | S470-F572 | |
| | | | | | LIM domain signature: C23-L56, C82-F115, | MOTIFS |
| | | | | | C151-L185, C210-F243 | |
| 18 | 489786CD1 | 1056 | S39 S84 S136 S361 | N134 N465 N486 | Ank repeat: E40-N72, C437-D469, K205-V237, | HMMER_PFAM |
| | | | S394 S427 S481 | N631 N1020 | M73-K105, D338-I370, A941-E973, N271-K304, | |
| | | | S526 S557 S589 | N1049 | S871-K904, L702-S734, A566-E598, T633-V665, | |
| | | | S761 S833 S871 | | Q771-F800, D905-E937, W470-K530, H371-K403, | |
| | | | S1051 T200 T365 | | E531-S564, N803-D837, T7-S39, G139-K171, | |
| | | | T602 T633 T713 | | Y238-N270, K838-N870, K669-I701, K599-N631, | |
| | | | T900 | | K172-K204, F404-K436, N106-R138 D305-K337 | |
| | | | | | R735-N770 | |
| | | | | | Ank repeat proteins. PF00023: L177-L192, G438-A447 | BLIMPS_PFAM |
| | | | | | ATP/GTP-binding site motif A (P-loop): A867-T874 | MOTIFS |
| 19 | 2240034CD1 | 923 | S7 S16 S25 S61 | N85 N701 N781 | WD domain, G-beta repeat: P768-K805, R655-D691, | HMMER_PFAM |
| | | | S67 S73 S94 S113 | N787 N815 N849 | F526-N562, I443-D479, L609-V645, T243-D277, | |
| | | | S118 S184 S314 | | I391-S425, K343-T379, L568-D603 | |
| | | | S596 S654 S769 | | | |
| | | | S829 S880 T143 | | | |
| | | | T243 T334 T349 | | | |
| | | | T386 T427 T496 | | | |
| | | | T512 T521 T667 | | | |
| | | | T785 T817 T851 | | | |
| | | | T863 T911 Y207 | | | |
| | | | Y713 | | | |
| | | | | | G-protein beta WD-40 repeat signature PR00320: | BLIMPS_{—} |
| | | | | | L549-S563, I792-L806, L466-H480 | PRINTS |
| | | | | | Pterin 4 alpha carbinola PF01329: Q803-K809, | BLIMPS_PFAM |
| | | | | | W561-P585 | |
| | | | | | PROTEIN HUEMAP ECHINODERM | BLAST_{—} |
| | | | | | MICROTUBULE-ASSOCIATED F38A6.2 | PRODOM |
| | | | | | PD021346: P141-V285 PD040953: R286-H374 | |
| | | | | | HUEMAP ECHINODERM MICROTUBULE- | BLAST_{—} |
| | | | | | ASSOCIATED PROTEIN PD024545: R486-W599 | PRODOM |
| | | | | | PD154836: C696-A767 | |
| | | | | | do MSI1; MEMBRANE; REPETITIVE; YDR128W; | BLAST_DOMO |
| | | | | | DM00299\|A55665\|530-575: G650-K697 | |
| | | | | | BETA-TRANSDUCIN FAMILY TRP-ASP | BLAST_DOMO |
| | | | | | REPEATS DM00005\|A55665\|181-231: S290-K340 | |
| | | | | | 482-529: L602-E648 132-180: E240-D289 | |
| | | | | | EF-hand calcium-binding domain: D326-W338 | MOTIFS |
| | | | | | Histidine acid phosphatases active site signature: | MOTIFS |
| | | | | | F631-S647 | |
| | | | | | Serine proteases, trypsin family, serine active site: | MOTIFS |
| | | | | | D411-L422 | |
| | | | | | Trp-Asp (WD) repeats signature: L549-S563 | MOTIFS |
| 20 | 3438037CD1 | 201 | S23 S33 S112 S190 | | Helix-loop-helix DNA-binding domain: Q76-G128 | HMMER_PFAM |
| | | | T5 T116 | | | |
| | | | | | Myc-type, ‘helix-loop-helix’ dimerization domain | BLIMPS_{—} |
| | | | | | proteins BL00038: E84-R99, D108-G128 | BLOCKS |
| | | | | | Myc-type, ‘helix-loop-helix’ dimerization domain | PROFILESCAN |
| | | | | | signature: N92-A148 | |
| | | | | | Wilm's tumor protein signature PR00049: G62-H78, | BLIMPS_{—} |
| | | | | | T5-V19, A152-P166 | PRINTS |
| | | | | | Vinculin signature PR00806: P158-E171, G156-P166 | BLIMPS_{—} |
| | | | | | PRINTS | |
| | | | | | SCLERAXIS PD056833: G128-S201 PD050814: | BLAST_{—} |
| | | | | | M1-Q50 | PRODOM |
| | | | | | MYC-TYPE, ‘HELIX-LOOP-HELIX’ | BLAST_DOMO |
| | | | | | DIMERIZATION DOMAIN DM00051\|A57717\|57-150: | |
| | | | | | R59-C143 \|P26687\|93-190: G57-E135 | |
| | | | | | \|Q02575\|38-133: G57-L131 \|P12980\|101-233: | |
| | | | | | G67-P147 | |
| | | | | | Myc-type, ‘helix-loop-helix’ dimerization domain | MOTIFS |
| | | | | | signature: S112-L127 | |
| 21 | 6578021CD1 | 793 | S53 S58 S68 S101 | N52 N302 N330 | WD domain, G-beta repeat: F486-D522, P728-R765, | HMMER_PFAM |
| | | | S112 S132 S265 | N340 | R615-D651, V570-S605, K294-N330, G194-D228, | |
| | | | S285 S300 S321 | | I403-G439 | |
| | | | S385 S613 S700 | | | |
| | | | S741 S789 T16 T48 | | | |
| | | | T344 T472 T556 | | | |
| | | | T627 T745 Y172 | | | |
| | | | Y591 Y673 | | | |
| | | | | | HUEMAP ECHINODERM MICROTUBULE- | BLAST_{—} |
| | | | | | ASSOCIATED PROTEIN PD024545: L445-W559 | PRODOM |
| | | | | | PD154836: C656-A727 | |
| | | | | | do MSI1; MEMBRANE; REPETITIVE; YDR128W; | BLAST_DOMO |
| | | | | | DM00299\|A55665\|530-575: G610-K657 | |
| | | | | | Cell attachment sequence: R479-D481 | MOTIFS |
| 22 | 8013295CD1 | 1094 | S127 S160 S314 | N81 N312 N315 | Ank repeat: K185-K217, M350-K382, S53-V85, | HMMER_PFAM |
| | | | S374 S409 S431 | N526 N878 | S218-K250, Q284-K316, N251-R283, N20-E52, | |
| | | | S468 S513 S519 | N1033 | S152-K184, S317-K349, R86-K118, N383-V415, | |
| | | | S559 S596 S643 | | D119-T151 | |
| | | | S669 S695 S713 | | | |
| | | | S765 S880 S927 | | | |
| | | | S989 S998 S1050 | | | |
| | | | S1061 S1089 T182 | | | |
| | | | T183 T270 T435 | | | |
| | | | T448 T450 T460 | | | |
| | | | T480 T615 T621 | | | |
| | | | T687 T772 T1004 | | | |
| | | | T1020 T1045 | | | |
| | | | | | ZU5 domain: S582-E686 | HMMER_PFAM |
| | | | | | Domain present in ZO-1 a PF00791A: Q372-R426 | BLIMPS_PFAM |
| | | | | | PF00791B: L25-S79 PF00791C: L204-G242 | |
| | | | | | PF00791D: L703-N745 PF00791E: L771-P823 | |
| | | | | | PF00791F: E992-K1016 PF00791G: V1029-K1044 | |
| | | | | | Ank repeat proteins. PF00023: L91-L106, G318-Q327 | BLIMPS_PFAM |
| | | | | | ANKYRIN REPEAT UNC44 CYTOSKELETON | BLAST_{—} |
| | | | | | ANK ELEGANS ANKYRIN-RELATED GB: | PRODOM |
| | | | | | U21734 ALTERNATIVE SPLICING | |
| | | | | | PD004427: W688-T1074 | |
| | | | | | ANKYRIN REPEAT CYTOSKELETON ANK | BLAST_{—} |
| | | | | | ALTERNATIVE SPLICING PHOSPHORYLATION | PRODOM |
| | | | | | MULTIGENE FAMILY LIPOPROTEIN PD149987: | |
| | | | | | T381-H581 | |
| | | | | | ANKYRIN REPEAT ELEGANS ALTERNATIVE | BLAST_{—} |
| | | | | | SPLICING UNC44 C CYTOSKELETON ANK | PRODOM |
| | | | | | PHOSPHORYLATION PD003245: S582-E686 | |
| | | | | | UNC44 ELEGANS ANKYRINRELATED GB: | BLAST_{—} |
| | | | | | U21734 C ANKYRINSE PD014451: E412-A475 | PRODOM |
| | | | | | ANKYRIN DM03652 \|A55575\|818-1423: S431-G1023 | BLAST_DOMO |
| | | | | | \|Q01484\|816-1438: V439-Q1049 \|P16157\|788-1366: | |
| | | | | | M536-P1037 \|I49502\|785-1363: E538-P1037 | |
| 23 | 5001859CD1 | 818 | S48 S49 S50 S96 | N25 N387 N567 | PH domain: L354-G447, A176-G271 | HMMER_PFAM |
| | | | S133 S143 S147 | N659 | | |
| | | | S208 S303 S309 | | | |
| | | | S328 S344 S374 | | | |
| | | | S414 S431 S448 | | | |
| | | | S484 S533 S563 | | | |
| | | | S607 S627 S728 | | | |
| | | | S736 S767 S777 | | | |
| | | | T12 T345 T450 | | | |
| | | | T488 T581 T612 | | | |
| | | | T650 T661 T669 | | | |
| | | | T704 T756 T761 | | | |
| | | | T789 T800 Y359 | | | |
| | | | Y457 Y489 Y537 | | | |
| | | | Y668 | | | |
| | | | | | Bacillus thuringiensist PF01338: R233-L272, N219-I266 | BLIMPS_PFAM |
| | | | | | ACTIN PROTEIN FILAMENT-ASSOCIATED | BLAST_{—} |
| | | | | | FILAMENT PD043475: P107-Y489 PD043476: | PRODOM |
| | | | | | R646-R730 | |
| | | | | | do KINASE; FILAMENT; TYROSINE; 110K; | BLAST_DOMO |
| | | | | | DM05530\|A54592\|1-85: L7-P86 | |
| 24 | 7506133CD1 | 617 | S24 S219 S228 | N288 N327 N372 | signal_cleavage: M1-A67 | SPSCAN |
| | | | S250 S256 S359 | N391 | | |
| | | | S420 S436 S468 | | | |
| | | | S481 S508 S543 | | | |
| | | | T32 T128 T295 | | | |
| | | | T477 | | | |
| | | | | | PH domain: I278-R375 | HMMER_PFAM |
| 25 | 5301066CD1 | 402 | S97 S103 S113 | | Villin headpiece domain: H367-F402 | HMMER_PFAM |
| | | | S183 S236 S279 | | | |
| | | | S301 S369 S380 | | | |
| | | | T35 T136 T281 | | | |
| | | | T348 T362 Y67 | | | |
| | | | | | PROTEIN DEMATIN ACTIN BINDING | BLAST_{—} |
| | | | | | ERYTHROCYTE MEMBRANE BAND CAPPING | PRODOM |
| | | | | | REPEAT PHOSPHORYLATION A PD017047: | |
| | | | | | E27-D246 | |
| | | | | | PROTEIN ACTIN BINDING CAPPING REPEAT | BLAST_{—} |
| | | | | | VILLIN CALCIUM PUTATIVE SUPER VILLIN | PRODOM |
| | | | | | DEMATIN ADVILLIN PD003485: E336-F402 | |
| | | | | | VILLIN HEADPIECE DM04001\|Q08495\|252-382: | BLAST_DOMO |
| | | | | | S301-F402 | |

TABLE 4

| Polynucleotide | |
|---|---|
| SEQ ID NO:/ | |
| Incyte ID/Sequence | |
| Length | Sequence Fragments |
| 26/2780338CB1/ | 1-746, 2-592, 4-662, 4-674, 4-732, 4-736, 4-749, 4-4103, 243-593, 243-933, 516-742, 574-1177, 576-810, 657-940, |
| 4519 | 657-1187, 667-1250, 714-1138, 815-1457, 815-1548, 834-1454, 857-1458, 857-1602, 857-1691, |
| | 876-1691, 906-1302, 911-1302, 911-1310, 1020-1486, 1020-1511, 1020-1560, 1020-1583, 1020-1607, 1020-1691, |
| | 1042-1691, 1070-1228, 1126-1564, 1130-1423, 1369-1691, 1384-2176, 1440-3065, 1486-2086, 1570-1850, 1733-2176, |
| | 1822-2389, 1822-2438, 1825-2403, 1965-2645, 2022-2645, 2041-2645, 2208-2639, 2230-2462, 2230-2771, 2364-2569, |
| | 2379-3143, 2547-3116, 2563-3233, 2588-3024, 2689-3269, 2734-2982, 2734-3224, 2814-3322, |
| | 2824-3509, 2885-3510, 2891-3509, 2894-3729, 2918-3510, 2928-3510, 2997-3785, 3004-3509, 3253-3828, 3336-3744, |
| | 3375-3879, 3417-3872, 3421-4003, 3508-4072, 3526-4092, 3580-4101, 3653-4025, 3654-4107, |
| | 3730-3998, 3751-4273, 3757-4237, 3771-4375, 3806-4464, 3837-4094, 3914-4462, 3967-4268, 3967-4273, 3967-4519, |
| | 3968-4510, 3993-4513, 4125-4418, 4239-4422, 4260-4519 |
| 27/2317440CB1/ | 1-620, 1-665, 1-668, 120-723, 122-723, 262-1151, 708-1151, 1083-1412, 1311-1900, 1355-1987, 1697-1970, 1697-1979, |
| 8008 | 1697-2186, 1697-2209, 1697-2220, 1697-2259, 1697-2279, 1697-2283, 1697-2289, 1697-2326, 1697-2342, |
| | 1828-2395, 1829-2445, 1929-2526, 1957-2583, 2039-2586, 2050-2603, 2068-2445, 2101-2837, 2105-2721, 2124-2715, |
| | 2133-2763, 2144-2764, 2182-2537, 2200-2726, 2208-2854, 2243-2946, 2252-2837, 2261-2808, 2273-2328, |
| | 2281-2932, 2289-2606, 2295-2786, 2299-2916, 2303-2888, 2305-2808, 2308-2778, 2308-2858, 2315-2890, 2324-2924, |
| | 2331-2836, 2336-2836, 2341-2975, 2344-2730, 2356-3030, 2358-2667, 2367-2991, 2371-2901, 2374-2822, |
| | 2375-3095, 2392-2871, 2392-2916, 2393-3008, 2394-2974, 2395-3043, 2435-3007, 2459-3013, 2502-3074, 2503-3087, |
| | 2515-3109, 2593-3192, 2602-3030, 2602-3093, 2626-3204, 2629-3265, 2647-3254, 2698-3221, 2705-3100, |
| | 2727-3312, 2740-3182, 2744-3357, 2770-3412, 2771-3255, 2780-3354, 2797-3138, 2797-3401, 2827-3465, 2852-3386, |
| | 2854-3457, 2855-3279, 2896-3359, 2899-3413, 2911-3376, 2916-3361, 2941-3515, 2964-3581, 2967-3745, |
| | 2970-3745, 2979-3613, 3020-3518, 3035-3719, 3063-3745, 3067-3603, 3070-3739, 3094-3730, 3124-3739, |
| | 3132-3745, 3145-3739, 3148-3660, 3158-3739, 3177-3739, 3183-3739, 3186-3739, 3191-3739, 3202-3739, 3210-3738, |
| | 3218-3739, 3229-3746, 3230-3739, 3231-3739, 3251-3749, 3280-3739, 3281-3739, 3297-3739, 3301-3739, |
| | 3307-3739, 3311-3739, 3334-3485, 3424-3785, 3741-4081, 3742-4159, 3749-4309, 3798-4324, 4189-4719, 4189-4824, |
| | 4268-4969, 4434-4988, 4720-4988, 4826-5420, 4985-5620, 5079-8008, 5171-5777, 5210-5852, 5324-6013, |
| | 5472-5986, 5496-5852, 5535-5884, 5601-5871, 5721-6280, 5802-6035, 5853-6351, 5855-6120, 5859-6035, 5859-6358, |
| | 5859-6369, 5859-6447, 5859-6456, 5859-6506, 5898-6316, 5910-6564, 5915-6440, 5923-6535, 5924-6241, |
| | 5924-6282, 5924-6301, 5924-6453, 5924-6493, 5924-6542, 5965-6369, 5984-6664, 6053-6616, 6101-6350, 6150-6732, |
| | 6151-6710, 6163-6657, 6190-6821, 6200-6502, 6237-6837, 6247-6443, 6268-6817, 6301-6854, 6301-6860, |
| | 6339-6973, 6341-6552, 6344-6836, 6355-6786, 6364-6913, 6367-6912, 6388-7050, 6394-6639, 6401-7024, 6417-6947, |
| | 6421-6981, 6424-7065, 6426-6982, 6429-6833, 6450-6985, 6464-6962, 6464-7203, 6465-6988, 6495-7022, |
| | 6503-7056, 6516-6952, 6518-6941, 6566-7070, 6571-7070, 6577-7256, 6579-7310, 6584-7107, 6590-7222, |
| | 6614-7166, 6627-7189, 6638-7385, 6662-7316, 6663-7272, 6671-7376, 6691-7376, 6706-7195, 6706-7272, 6725-7340, |
| | 6745-7376, 6749-7296, 6751-7299, 6758-7276, 6776-7420, 6777-7424, 6779-7274, 6818-7301, 6821-6977, |
| | 6837-7524, 6922-7511, 6931-7432, 6980-7524, 6986-7545, 7040-7631, 7061-7726, 7068-7218, 7099-7684, 7160-7422, |
| | 7193-7461, 7195-7663, 7207-7432, 7221-7821, 7255-7471, 7273-7627, 7316-7916, 7363-7786, 7367-7782, |
| | 7367-7928, 7387-7592, 7395-7645, 7528-7822, 7561-7928, 7596-7865, 7631-7872, 7632-7928, 7637-7927, 7646-7674, |
| | 7671-7916, 7673-7928, 7735-7928, 7735-7929, 7741-7928, 7794-7928, 7816-7928, 7817-7928, 7829-7928, |
| | 7842-7928 |
| 28/3013470CB1/ | 1-479, 2-173, 34-583, 37-557, 53-508, 76-410, 292-724, 311-499, 600-1203, 692-1303, 778-1050, 778-1203, 778-1301, |
| 2160 | 778-1321, 778-1337, 840-1130, 840-1137, 840-1402, 864-1388, 884-1053, 948-1596, 989-1472, 1029-1241, |
| | 1055-1288, 1073-1595, 1083-1761, 1095-1385, 1133-1739, 1135-1621, 1137-1703, 1148-1743, 1157-1753, 1197-1827, |
| | 1199-1824, 1217-1867, 1240-1445, 1286-1519, 1286-1668, 1287-1733, 1290-1554, 1338-1948, 1344-2012, |
| | 1346-1955, 1347-1909, 1361-1768, 1370-2035, 1381-1646, 1412-1935, 1427-1916, 1430-2070, 1432-1975, 1449-2000, |
| | 1449-2058, 1458-2046, 1475-2110, 1481-2140, 1496-2106, 1503-2120, 1544-2160, 1577-2112, 1599-2121, |
| | 1608-2119, 1616-2120, 1629-2121, 1640-1901, 1673-2149, 1708-1908, 1729-2048, 1796-2104, 1796-2121, 1802-2119, |
| | 1906-2121, 1910-2016, 1910-2112, 1910-2121 |
| 29/1738823CB1/ | 1-405, 51-464, 63-365, 73-405, 122-382, 250-882, 406-616, 509-629, 542-649, 542-809, 542-832, 542-1032, 618-1151, |
| 2992 | 702-1337, 731-1331, 796-1102, 797-1443, 820-1095, 820-1250, 888-1403, 903-1638, 912-1473, 954-1756, |
| | 966-1356, |
| | 1028-1231, 1077-1280, 1077-1461, 1077-1667, 1171-1393, 1203-1270, 1209-1494, 1209-1513, 1270-1681, 1325-1675, |
| | 1333-1571, 1333-1659, 1333-1681, 1342-1592, 1342-1819, 1360-1634, 1443-1627, 1443-1681, 1454-1792, |
| | 1469-1681, |
| | 1470-1681, 1592-1897, 1604-1867, 1677-1946, 1746-2020, 1746-2320, 1772-2055, 1862-2123, 1869-2104, 1870-2089, |
| | 1897-2121, 1897-2449, 1941-2202, 1994-2223, 2013-2298, 2024-2262, 2024-2263, 2128-2415, 2217-2451, |
| | 2287-2516, |
| | 2287-2777, 2297-2551, 2311-2583, 2323-2588, 2334-2862, 2338-2599, 2340-2518, 2340-2538, 2388-2859, 2431-2862, |
| | 2448-2875, 2481-2697, 2484-2852, 2505-2875, 2506-2875, 2528-2875, 2544-2862, 2567-2875, |
| | 2571-2799, 2571-2875, 2605-2875, 2616-2875, 2728-2992, 2746-2875, 2753-2875 |
| 30/4184551CB1/ | 1-233, 1-245, 1-280, 1-619, 9-536, 26-622, 386-683, 386-839, 386-861, 389-866, 410-860, 427-868, 458-1036, 460-755, |
| 3225 | 511-866, 569-839, 588-857, 588-866, 591-859, 593-861, 600-1264, 621-892, 668-1145, 810-1234, 846-1441, |
| | 861-1068, 1095-1738, 1168-1708, 1187-1393, 1360-1634, 1373-1959, 1524-1616, 1638-2259, 1739-2274, 1742-2228, |
| | 1783-2475, 1892-2554, 1893-2171, 2073-3225, 2156-2553, 2159-2507, 2228-2553, 2265-2553, |
| | 2278-2559, 2589-2835, 2589-2960, 2589-3064, 2600-3057, 2637-3057 |
| 31/70042484CB1/ | 1-719, 12-485, 24-422, 32-412, 34-494, 78-488, 96-465, 101-412, 158-494, 431-1062, 495-842, 495-871, 495-911, |
| 2787 | 495-1099, 507-900, 516-904, 516-983, 540-625, 733-1246, 786-989, 822-930, 832-1147, 835-1446, 973-1370, 1074-1276, |
| | 1096-1664, 1116-1271, 1131-1220, 1158-1781, 1158-1782, 1164-1268, 1166-1963, 1176-1402, 1192-1383, |
| | 1265-1739, 1266-1727, 1280-1885, 1299-1924, 1342-1749, 1352-1898, 1358-1567, 1376-1941, 1417-1596, 1419-1974, |
| | 1421-1627, 1429-1929, 1435-2032, 1479-1822, 1494-1999, 1494-2042, 1506-2068, 1507-2116, 1535-2134, |
| | 1542-1886, 1558-1809, 1568-2118, 1629-2122, 1650-1892, 1656-2148, 1662-1988, 1671-2286, 1675-1994, 1682-2207, |
| | 1755-2255, 1761-2278, 1765-2138, 1766-2138, 1809-2034, 1820-2067, 1821-2056, 1822-2090, 1834-2492, |
| | 1850-2480, 1855-2097, 1855-2492, 1888-2397, 1897-2400, 1903-2099, 1923-2192, 1934-2356, 1934-2361, 1959-2250, |
| | 1975-2217, 1975-2492, 1975-2501, 1975-2502, 1975-2541, 1979-2067, 1983-2083, 1989-2545, 1992-2123, |
| | 1996-2484, 2000-2444, 2002-2231, 2033-2535, 2042-2543, 2048-2306, 2056-2505, 2062-2348, 2080-2320, 2080-2396, |
| | 2092-2503, 2137-2787, 2179-2354, 2181-2477, 2188-2502, 2196-2465, 2222-2489, 2222-2540, 2228-2508, |
| | 2234-2323 |
| 32/3236274CB1/ | 1-815, 1-826, 24-655, 33-349, 56-716, 71-324, 71-606, 112-692, 114-350, 134-593, 152-772, 260-709, 262-595, 335-761, |
| 2444 | 393-652, 457-719, 457-890, 462-844, 532-900, 536-715, 573-931, 573-1067, 602-840, 622-1121, |
| | 869-1129, 918-1274, 921-1403, 1065-1336, 1076-1354, 1162-1725, 1193-1367, 1347-1605, 1358-1650, 1381-1913, |
| | 1410-1982, 1413-2173, 1430-2102, 1442-1649, 1457-2009, 1537-1787, 1645-1871, 1659-2232, |
| | 1742-2396, 1811-1956, 1831-1956, 1838-2115, 1839-2071, 1894-2176, 1905-2444, 1909-2160 |
| 33/7179725CB1/ | 1-634, 429-1064, 476-925, 493-934, 644-1240, 742-1194, 935-1254, 960-1222, 979-1468, 993-1242, 1129-1417, |
| 2605 | 1131-1398, 1186-1417, 1207-1448, 1335-1853, 1360-1608, 1394-1590, 1398-1946, 1409-1694, 1417-1611, 1448-1688, |
| | 1457-1743, 1474-1741, 1476-1964, 1481-1640, 1487-1506, 1487-1531, 1519-1623, 1519-1640, 1519-1656, |
| | 1519-1720, 1519-1892, 1539-1625, 1547-1624, 1553-1597, 1563-1688, 1564-1622, 1578-1656, 1580-1624, 1580-1634, |
| | 1580-1640, 1580-1656, 1580-1892, 1588-1640, 1606-1660, 1606-1734, 1606-1868, 1611-1656, 1613-1688, |
| | 1616-1829, 1616-1830, 1616-1852, 1616-1853, 1616-1997, 1617-1853, 1619-1853, 1630-1688, 1630-1720, 1630-1728, |
| | 1630-1786, 1630-1787, 1630-1792, 1639-2123, 1646-1792, 1649-2189, 1668-1792, 1672-1800, 1672-1919, |
| | 1672-1934, 1672-1985, 1674-2183, 1687-2179, 1696-1946, 1706-1792, 1712-1899, 1712-1919, 1741-1815, 1741-1834, |
| | 1741-1849, 1741-1857, 1741-1890, 1745-1882, 1745-1895, 1745-1901, 1745-2018, 1776-1820, 1776-1849, |
| | 1776-1865, 1776-1933, 1776-1934, 1776-1948, 1777-1853, 1778-1885, 1778-1886, 1778-1901, 1778-1915, 1778-1934, |
| | 1778-1946, 1778-2018, 1778-2051, 1782-1948, 1796-1901, 1796-2162, 1798-2342, 1800-2369, 1802-1853, |
| | 1805-1833, 1805-1892, 1806-1868, 1808-1948, 1.811-1919, 1811-2018, 1811-2051, 1816-1937, 1816-1953, 1816-2189, |
| | 1818-2018, 1822-1897, 1822-1901, 1822-2018, 1826-1946, 1828-2052, 1828-2071, 1828-2078, 1833-1946, |
| | 1838-1886, 1838-1934, 1838-2051, 1844-1919, 1844-1923, 1844-1934, 1844-1939, 1844-1946, 1851-1886, 1851-1934, |
| | 1854-2041, 1854-2085, 1854-2092, 1854-2236, 1854-2240, 1854-2306, 1854-2359, 1854-2362, 1859-1934, |
| | 1871-1946, 1877-1953, 1887-2051, 1887-2066, 1894-1946, 1906-2570, 1910-2018, 1910-2078, 1919-2183, 1926-2566, |
| | 1934-2210, 1950-2565, 1957-2567, 1958-2071, 1958-2078, 1965-2541, 1967-2018, 1974-2018, 1975-2121, |
| | 1976-2053, 1980-2051, 1985-2239, 1985-2244, 1985-2574, 1993-2281, 1994-2263, 1998-2230, 1998-2369, 2003-2052, |
| | 2003-2071, 2003-2078, 2007-2051, 2025-2071, 2025-2078, 2026-2302, 2030-2308, 2053-2535, 2083-2295, |
| | 2083-2544, 2084-2113, 2084-2341, 2098-2302, 2099-2150, 2101-2195, 2101-2216, 2101-2219, 2102-2130, 2102-2151, |
| | 2106-2150, 2106-2178, 2106-2183, 2106-2193, 2106-2195, 2106-2278, 2107-2182, 2107-2183, 2107-2242, |
| | 2107-2250, 2107-2253, 2109-2294, 2111-2253, 2125-2219, 2125-2223, 2125-2287, 2125-2584, 2128-2584, |
| | 2132-2437, 2137-2583, 2138-2195, 2138-2275, 2138-2278, 2138-2584, 2139-2465, 2141-2287, 2146-2588, 2148-2586, |
| | 2157-2583, 2163-2287, 2165-2586, 2170-2256, 2170-2277, 2170-2278, 2170-2282, 2170-2287, 2170-2319, |
| | 2172-2282, |
| | 2183-2433, 2183-2459, 2192-2298, 2192-2586, 2205-2253, 2206-2249, 2206-2265, 2206-2282, 2206-2319, 2206-2338, |
| | 2206-2342, 2206-2352, 2207-2248, 2207-2254, 2207-2265, 2207-2287, 2207-2297, 2208-2352, 2210-2491, |
| | 2210-2526, 2214-2297, 2222-2286, 2222-2297, 2224-2247, 2233-2327, 2233-2351, 2240-2287, 2240-2298, 2240-2320, |
| | 2246-2590, 2272-2583, 2278-2597, 2300-2583, 2304-2352, 2305-2600, 2312-2342, 2312-2352, 2318-2584, |
| | 2328-2605, 2335-2584, 2346-2512, 2375-2550, 2382-2577, 2382-2603 |
| 34/1966217CB1/ | 1-750, 389-1519, 479-790, 479-878, 515-989, 605-919, 745-1136, 989-1180, 1114-1747, 1114-1818, 1114-1849, |
| 3716 | 1114-1872, 1114-1920, 1114-1982, 1186-1438, 1186-1859, 1287-1830, 1290-1558, 1351-1716, 1354-1714, 1416-1631, |
| | 1451-2066, 1475-1724, 1475-1730, 1475-1758, 1475-1898, 1475-1908, 1475-1923, 1475-1943, 1475-1962, |
| | 1475-1993, 1475-2038, 1475-2050, 1528-2226, 1542-2027, 1570-1967, 1622-1839, 1627-1987, 1637-2167, 1643-2066, |
| | 1675-1995, 1682-2050, 1793-2390, 1849-2264, 1896-2227, 1943-2190, 1944-2541, 1968-2587, 1984-2485, |
| | 2024-2151, 2037-2792, 2111-2362, 2111-2643, 2128-2634, 2130-2609, 2200-2634, 2201-2290, 2219-2867, 2225-2866, |
| | 2266-2513, 2266-2706, 2276-2825, 2279-2883, 2291-2596, 2313-2529, 2331-2836, 2337-2881, 2381-2899, |
| | 2430-2895, 2432-2895, 2448-2950, 2455-2609, 2487-2899, 2546-2897, 2549-2895, 2567-2980, 2568-2710, 2600-2899, |
| | 2628-2903, 2694-2895, 2866-3096, 2866-3326, 2867-3335, 2902-3134, 2903-3094, 2903-3103, 2903-3134, |
| | 2903-3135, 2903-3137, 2976-3588, 3098-3618, 3098-3667, 3398-3592, 3398-3682, 3402-3716 |
| 35/1598186CB1/ | 1-30, 1-32, 1-33, 1-35, 1-36, 1-68, 1-79, 33-83, 34-79, 36-79, 37-78, 37-79, 37-83, 37-408, 37-454, 39-78, 39-79, |
| 1372 | 133-421, 136-406, 187-275, 187-286, 187-319, 187-327, 187-366, 187-411, 187-538, 187-547, 187-553, |
| | 187-562, 187-583, 187-633, 187-642, 187-661, 187-769, 187-890, 187-1372, 191-696, 193-384, 201-360, 350-641, |
| | 384-600, 409-560, 512-932, 525-1039, 558-780, 564-843, 595-1104, 613-840, 617-905, 629-887, 638-727, |
| | 695-829, 727-962, 730-994, 748-867, 749-926, 749-1081, 751-996, 754-1005, 769-1002, 769-1019, 782-1051, 809-1094, |
| | 811-1096, 822-1067, 830-1085, 840-1118, 850-1099, 869-1171, 873-1156, 887-1104, 887-1154, |
| | 888-1172, 889-1172, 920-1139, 922-1172, 942-1172, 954-1172, 955-1172, 956-1172, 1004-1172, 1014-1172, 1021-1171, |
| | 1032-1172, 1047-1172, 1059-1172, 1062-1172, 1064-1172, 1070-1172, 1075-1172, 1091-1172, 1094-1172, |
| | 1101-1172 |
| 36/7493044CB1/ | 1-526, 151-526, 434-1465, 1307-1562 |
| 1562 | |
| 37/7925017CB1/ | 1-4249, 101-819, 751-977, 828-978, 845-1437, 964-1514, 966-1488, 1126-1519, 1214-4296, 1520-1558, 2003-2225, |
| 4296 | 2094-2225 |
| 38/6758789CB1/ | 1-204, 1-367, 88-367, 224-658, 439-1057, 596-751, 611-751, 810-1506, 887-1074, 889-1156, 894-1177, 894-1178, |
| 2045 | 894-1224, 1001-1595, 1011-1402, 1011-1606, 1011-1608; 1011-1625, 1011-1641, 1011-1651, |
| | 1011-1666, 1020-1275, 1020-1384, 1152-1373, 1186-1384, 1225-1490, 1228-1826, 1242-1384, 1481-1898, 1583-2044, |
| | 1583-2045, 1585-2045, 1637-2045, 1638-2001, 1691-2045, 1701-1895, 1735-2045 |
| 39/7488249CB1/ | 1-435, 130-448, 179-413, 341-558, 372-995, 387-641, 387-939, 388-961, 388-1096, 395-558, 395-972, 532-1043, |
| 1919 | 533-1043, 543-1043, 545-966, 545-1006, 545-1043, 549-1039, 574-1170, 578-1043, 627-1200, 642-751, 645-1292, |
| | 649-1325, 677-1091, 679-1148, 713-1229, 731-1270, 765-1274, 791-1343, 793-1241, 795-1174, 805-923, 855-1463, |
| | 872-1157, 926-1314, 933-1522, 939-1089, 973-1468, 982-1433, 1006-1584, 1076-1529, 1129-1388, 1192-1583, |
| | 1265-1919, 1301-1791, 1335-1770, 1407-1593 |
| 40/5046311CB1/ | 1-290, 2-638, 15-493, 17-268, 23-321, 54-287, 65-310, 69-372, 83-394, 83-817, 85-671, 102-633, 202-501, 203-311, |
| 5923 | 211-688, 242-541, 245-713, 245-750, 245-814, 245-824, 245-875, 245-878, 245-924, 245-925, 245-976, 245-979, |
| | 253-495, 253-752, 348-1040, 353-817, 398-976, 451-1034, 474-906, 478-1034, 480-847, 516-863, 517-2094, 531-1156, |
| | 544-668, 547-822, 547-878, 547-1027, 547-1091, 547-1119, 547-1153, 547-1180, 547-1274, 548-847, 555-744, |
| | 555-1143, 558-1058, 578-1135, 589-1113, 592-1132, 604-1018, 617-1158, 650-1158, 651-1158, 652-1149, 658-1141, |
| | 682-1090, 682-1149, 693-1158, 703-1147, 726-1330, 760-1164, 799-1324, 890-1428, 904-1764, 933-1675, |
| | 938-1412, 947-1479, 990-1266, 1006-1904, 1037-1629, 1038-1149, 1041-1851, 1055-1676, 1112-1503, 1112-1569, |
| | 1134-1752, 1155-5923, 1183-1399, 1189-1408, 1189-1471, 1219-1430, 1219-1457, 1226-1967, 1228-1998, 1272-1471, |
| | 1319-1427, 1319-2003, 1327-1998, 1359-2119, 1360-2003, 1365-2003, 1371-2003, 1382-2003, 1413-2003, |
| | 1415-1923, 1427-2003, 1437-2003, 1441-2003, 1469-2003, 1476-1973, 1545-2003, 1567-2003, 1624-2003, 1635-2003, |
| | 1640-2003, 1651-2003, 1886-2018, 1983-2791, 2264-2644, 2303-2644, 2333-2644, 2334-2644, |
| | 2361-2644, 2424-2644, 2441-2644, 2545-2644, 2603-3134, 2659-3246, 2900-3563, 2923-3417, 2968-3425 |
| 41/931056CB1/2789 | 1-561, 20-551, 20-593, 20-594, 38-181, 52-2773, 103-592, 122-746, 346-534, 347-521, 347-686, 347-691, 347-692, |
| | 347-715, 347-735, 347-773, 347-788, 347-810, 347-853, 347-854, 347-899, 347-951, 347-996, 347-1047, |
| | 348-609, 360-594, 446-766, 472-1166, 481-1132, 493-594, 523-1222, 1162-1760, 1498-2115, 1640-2104, 1647-2198, |
| | 1693-1911, 1984-2166, 1984-2219, 1984-2310, 1984-2379, 1984-2563, 1985-2619, 2094-2782, 2105-2695, |
| | 2105-2781, 2105-2782, 2105-2783, 2108-2292, 2109-2692, 2125-2694, 2128-2281, 2137-2771, 2164-2766, 2195-2495, |
| | 2202-2783, 2295-2784, 2307-2565, 2322-2772, 2349-2769, 2350-2783, 2354-2769, 2364-2698, |
| | 2548-2768, 2554-2769, 2654-2772, 2707-2786, 2707-2789, 2709-2789, 2712-2789 |
| 42/2578937CB1/ | 1-663, 12-675, 38-612, 38-636, 38-673, 60-728, 65-654, 100-764, 101-769, 101-816, 145-860, 147-824, 149-700, |
| 4077 | 227-665, 237-1111, 244-793, 246-796, 253-704, 254-704, 323-1055, 340-1052, 354-997, 371-596, 382-983, 389-993, |
| | 390-849, 396-716, 423-844, 425-625, 426-1131, 434-1111, 450-945, 460-842, 492-790, 505-1058, 510-1020, |
| | 520-1103, 534-1133, 537-970, 542-1069, 561-1295, 581-1083, 601-853, 655-1306, 999-1301, 1004-1564, 1062-1299, |
| | 1080-1325, 1175-1833, 1191-1805, 1266-1530, 1389-1945, 1398-1732, 1530-2189, 1537-2084, 1543-2059, |
| | 1578-2080, 1704-2180, 1742-2456, 1750-2097, 1840-1908, 1840-2057, 1840-2320, 1840-2322, 1852-2019, 1864-2554, |
| | 1896-2407, 1914-2484, 1929-2164, 1929-2295, 1930-2536, 1984-2526, 1985-2433, 1995-2353, 2026-2451, |
| | 2026-2574, 2026-2610, 2040-2535, 2042-2535, 2045-2270, 2045-2313, 2060-2378, 2087-2355, 2098-2714, 2121-2575, |
| | 2173-2654, 2265-2538, 2265-2546, 2312-2583, 2312-2815, 2347-2565, 2347-2601, 2380-2534, 2404-2687, |
| | 2409-2986, 2435-2699, 2464-2761, 2509-2793, 2512-2803, 2597-2862, 2597-3053, 2638-2928, 2840-3402, 2843-3088, |
| | 2869-3091, 2925-3176, 2925-3465, 2932-3193, 2980-3239, 3036-3291, 3036-3542, 3051-3315, 3072-3341, |
| | 3109-3414, 3215-3442, 3228-3428, 3228-3460, 3228-3488, 3228-3546, 3228-3741, 3268-3546, 3293-3560, 3412-3679, |
| | 3427-4048, 3434-4027, 3446-3735, 3449-4060, 3475-4060, 3482-4058, 3554-4055, 3570-4023, |
| | 3591-3845, 3631-3867, 3666-3924, 3666-4031, 3666-4066, 3713-3928, 3838-4077 |
| 43/489786CB1/4935 | 1-262, 1-578, 1-598, 1-666, 1-678, 6-330, 6-705, 18-426, 19-249, 19-692, 20-578, 23-321, 42-519, 47-582, 70-352, |
| | 72-492, 86-788, 116-569, 126-707, 129-702, 158-278, 163-706, 177-1802, 180-456, 326-1045, 329-1073, 331-1144, |
| | 362-808, 373-1069, 380-1157, 445-710, 452-1036, 462-1125, 575-1036, 584-1247, 637-1278, 638-1176, 649-1234, |
| | 674-1036, 689-1252, 704-1252, 716-1252, 742-1131, 742-1443, 745-1073, 745-1098, 750-1175, 750-1193, 750-1294, |
| | 822-1587, 845-1508, 848-1252, 851-1036, 855-1120, 855-1121, 855-1252, 874-1087, 883-1252, 894-1036, |
| | 911-1802, 922-1158, 1064-1798, 1093-1252, 1159-1784, 1187-1800, 1199-1795, 1251-1677, 1301-1800, 1336-1601, |
| | 1450-1700, 1464-1492, 1546-2028, 1546-2230, 1614-1879, 1673-1930, 1673-1968, 1707-2247, 1713-2432, |
| | 1903-2552, 1946-2190, 1946-2463, 2060-2691, 2333-2603, 2333-2616, 2358-2386, 2386-2612, 2386-2805, 2406-2962, |
| | 2661-3075, 2669-3075, 2907-3192, 3017-3551, 3056-3328, 3076-3295, 3076-3392, 3076-3504, 3076-3563, |
| | 3110-3442, 3175-3564, 3265-3951, 3306-3572, 3355-3923, 3358-3581, 3359-3598, 3364-3951, 3369-3985, 3381-3694, |
| | 3399-3860, 3495-3731, 3620-3904, 3741-3998, 3741-4225, 3741-4283, 3743-3926, 3832-4123, 3900-4246, |
| | 3908-4374, 3964-4145, 4046-4335, 4142-4381, 4246-4460, 4262-4690, 4301-4614, 4405-4869, 4434-4467, 4442-4467, |
| | 4496-4853, 4505-4859, 4531-4853, 4565-4817, 4606-4856, 4697-4853, 4703-4935, 4716-4860, 4760-4932 |
| 44/2240034CB1/ | 1-180, 1-269, 1-277, 1-290, 1-391, 1-402, 1-409, 1-477, 1-484, 1-485, 1-631, 3-391, 5-496, 10-448, 10-473, 28-317, |
| 3400 | 28-365, 28-483, 48-300, 49-347, 49-450, 51-482, 65-454, 70-478, 84-502, 322-1005, 414-920, 499-1016, 595-1176, |
| | 624-898, 643-898, 693-1250, 1028-1649, 1029-1242, 1029-1498, 1029-1518, 1035-1209, 1062-1689, 1081-1311, |
| | 1112-1669, 1132-1738, 1154-1738, 1202-1484, 1218-1743, 1234-1797, 1283-1839, 1283-1938, 1325-1743, 1332-1897, |
| | 1337-1743, 1475-1848, 1475-1902, 1491-1748, 1505-1743, 1517-1927, 1528-2193, 1600-1743, 1639-2292, |
| | 1679-2211, 1684-2250, 1753-2317, 1894-2438, 1961-2244, 1994-2558, 2029-2646, 2095-2686, 2099-2621, 2109-2408, |
| | 2116-2400, 2179-2898, 2187-2425, 2202-2393, 2202-2413, 2202-2750, 2226-2489, 2243-2926, 2350-2960, |
| | 2391-2902, 2512-3187, 2569-2842, 2588-3228, 2659-2986, 2673-2912, 2680-3290, 2683-2960, 2683-3018, 2712-3016, |
| | 2713-3159, 2713-3161, 2735-3243, 2760-2956, 2760-2984, 2781-3068, 2782-3056, 2794-3214, 2808-3400, |
| | 2809-3247, 2811-2908, 2811-3065, 2818-3386, 2943-2975, 2943-3043, 2943-3080, 2943-3087, 2943-3339 |
| 45/3438037CB1/981 | 1-216, 14-449, 26-162, 323-551, 437-981, 456-656 |
| 46/6578021CB1/ | 1-444, 268-836, 268-1063, 268-2823, 273-784, 273-879, 273-932, 315-944, 315-1023, 328-998, 346-1125, 608-1019, |
| 3097 | 624-1094, 627-914, 748-1042, 797-1151, 801-1318, 870-1460, 884-1535, 941-1351, 945-1590, 957-1633, 998-1709, |
| | 1010-1180, 1014-1679, 1021-1627, 1026-1725, 1073-1352, 1112-1413, 1117-1568, 1123-1417, 1123-1622, |
| | 1123-1807, 1125-1686, 1138-1828, 1180-1246, 1304-1851, 1304-1912, 1304-1934, 1304-2081, 1356-1593, 1419-2140 |
| | 1485-1944, 1504-2129, 1508-2079, 1593-1806, 1593-2098, 1611-1869, 1611-2194, 1620-2128, 1638-1931, |
| | 1653-2226, 1688-1940, 1688-2096, 1710-2165, 1710-2166, 1743-2004, 1743-2235, 1744-2294, 1749-1973, 1766-2013, |
| | 1766-2389, 1772-1983, 1772-1999, 1802-2097, 1831-2129, 1835-2327, 1835-2435, 1855-2173, 1890-2145, |
| | 1898-2322, 1907-2092, 1911-2188, 1911-2189, 1911-2363, 1950-2121, 1954-2211, 1954-2466, 1979-2245, 2004-2236, |
| | 2004-2468, 2075-2357, 2075-2420, 2127-2398, 2136-2208, 2164-2712, 2182-2763, 2186-2909, 2228-2836, |
| | 2236-2499, 2278-2870, 2288-2534, 2288-2536, 2292-2758, 2301-2837, 2311-2809, 2376-2624, 2383-2610, 2391-2660, |
| | 2399-2658, 2421-3097, 2445-2671, 2453-3096, 2459-3023, 2472-2695, 2494-2687, 2494-2782, |
| | 2496-2785, 2498-2714, 2498-2951, 2500-2697, 2539-2866, 2589-2881, 2647-2912, 2862-3097 |
| 47/8013295CB1/ | 1-3472, 571-1090, 577-1195, 613-1186, 683-1216, 688-1309, 717-1314, 752-1125, 884-916, 970-1244, 1129-1831, |
| 3472 | 1149-1465, 1153-1465, 1225-1471, 1541-1622, 2396-2679, 2465-2665 |
| 48/5001859CB1/ | 1-620, 227-890, 256-558, 256-649, 256-955, 264-508, 264-698, 272-541, 272-547, 335-531, 335-1007, 358-901, 437-1011, |
| 5865 | 495-1133, 536-1278, 603-673, 673-1107, 776-1082, 853-1511, 968-1786, 1030-1292, 1030-1367, 1030-1489, |
| | 1030-1491, 1030-1515, 1030-1531, 1030-1562, 1030-1586, 1030-1589, 1031-1583, 1041-1640, 1087-1573, 1088-1681, |
| | 1102-1550, 1123-1650, 1172-1825, 1263-1479, 1292-1863, 1343-1920, 1364-1979, 1372-1933, 1373-1631, |
| | 1394-1855, 1404-2087, 1408-2009, 1410-1616, 1418-1737, 1447-2424, 1459-2125, 1468-2121, 1473-1764, 1543-1836, |
| | 1571-1718, 1578-2220, 1584-2271, 1597-2149, 1602-1871, 1656-2170, 1671-1951, 1683-2293, 1711-2301, |
| | 1719-1987, 1726-2287, 1733-1903, 1785-2257, 1797-2393, 1798-2253, 1798-2307, 1841-2462, 1851-2367, 1875-2472, |
| | 1890-2434, 1899-2280, 1899-2408, 1899-2515, 1900-2404, 1923-2330, 1924-2444, 1927-2563, 1929-2462, |
| | 1953-2376, 1973-2502, 1979-2483, 1998-2390, 2005-2649, 2007-2634, 2035-2569, 2094-2421, 2108-2421, 2125-2314, |
| | 2147-2373, 2164-2649, 2173-2805, 2176-2673, 2189-2402, 2192-2605, 2204-2788, 2205-2771, 2262-2598, |
| | 2272-2742, 2275-2576, 2288-2730, 2333-2842, 2368-2803, 2390-2939, 2391-2642, 2401-2919, 2437-2942, |
| | 2464-2942, 2496-2942, 2497-3101, 2527-2985, 2576-2939, 2592-3077, 2600-3091, 2606-2997, 2611-2853, 2618-3088, |
| | 2620-3008, 2638-2934, 2652-2831, 2652-3270, 2696-2964, 2697-2942, 2702-2996, 2714-2942, 2717-2917, |
| | 2717-3045, 2717-3157, 2717-3268, 2753-3347, 2806-3026, 2835-3125, 2881-3110, 2899-3155, 2919-3536, 2926-3524, |
| | 2958-3220, 2965-3537, 2988-3528, 2995-3352, 3000-3237, 3000-3524, 3005-3238, 3008-3534, 3033-3301, |
| | 3038-3392, 3093-3465, 3093-3545, 3108-3381, 3127-3546, 3131-3385, 3138-3545, 3143-3539, 3155-3539, 3171-3381 |
| | 3171-3715, 3177-3740, 3189-3444, 3193-3956, 3197-3453, 3197-3480, 3198-3538, 3227-3539, 3251-3568, |
| | 3253-3630, 3284-3510, 3284-3540, 3302-3583, 3309-3954, 3326-3751, 3337-3539, 3340-3933, 3343-3628, 3375-3951, |
| | 3388-3751, 3404-3628, 3435-3645, 3442-3645, 3466-3722, 3484-3772, 3519-3749, 3519-3770, 3519-3785, |
| | 3526-3955, 3563-3848, 3587-3959, 3593-3814, 3598-3927, 3692-3955, 3700-3937, 3770-3960, 3770-4223, 3770-4230, |
| | 3771-4115, 3773-3946, 3773-3952, 3774-4335, 3783-3961, 3786-4335, 3788-3958, 3995-4335, 4296-4668, |
| | 4296-4691, 4299-4527, 4299-4588, 4299-4664, 4303-4955, 4310-4866, 4314-4921, 4319-4960, 4368-4615, |
| | 4370-4885, 4370-4896, 4378-5146, 4429-4867, 4466-4952, 4519-4696, 4561-5152, 4704-5226, 4723-4952, 4837-5091, |
| | 4846-5282, 4875-5152, 4903-5138, 4917-5565, 5004-5631, 5049-5226, 5132-5379, 5137-5402, |
| | 5236-5473, 5270-5587, 5321-5865, 5405-5819, 5446-5631, 5468-5738, 5564-5819, 5615-5865 |
| 49/7506133CB1/ | 1-473, 1-815, 1-826, 1-2304, 24-655, 33-349, 56-716, 71-324, 71-606, 114-350, 134-593, 152-772, 260-709, 262-595, |
| 2340 | 268-623, 323-581, 393-652, 457-719, 457-890, 480-1067, 503-1123, 532-900, 536-715, 550-824, 573-931, 573-958, |
| | 602-840, |
| | 606-1209, 645-1181, 869-1129, 918-1274, 921-1180, 921-1403, 1019-1545, 1065-1336, 1076-1354, 1139-1774, |
| | 1237-1795, 1347-1605, 1358-1650, 1442-1649, 1517-1801, 1537-1787, 1543-1787, 1693-1956, 1707-2212, 1812-2049, |
| | 1812-2051, 1812-2088, 1812-2260, 1812-2283, 1824-2116, 1839-2092, 1839-2111, 1839-2116, 1856-2117, 1861-2114, |
| | 1862-2172, 1866-2139, 1886-2167, 1887-2070, 1906-2150, 1907-2081, 1907-2143, 1911-2185, 1915-2222, |
| | 1920-2184, 1925-2192, 1925-2201, 1937-2227, 1942-2197, 1944-2205, 1946-2153, 1961-2174, 1976-2273, 1980-2235, |
| | 1988-2114, 2018-2279, 2018-2340, 2021-2274, 2028-2224, 2077-2289, 2085-2321, 2090-2315, 2095-2325, |
| | 2112-2235, 2149-2329 |
| 50/5301066CB1/ | 1-588, 122-506, 243-588, 319-938, 326-609, 326-610, 326-656, 433-1027, 443-834, 443-1038, 443-1040, 443-1057, |
| 3263 | 443-1073, 443-1083, 443-1098, 452-707, 657-922, 660-1247, 910-989, 1185-1918, 1216-1239, 1381-1547, 1429-2307, |
| | 1614-2209, 1723-2256, 1730-2189, 1757-2292, 1758-2243, 1786-2285, 1800-2478, 1815-2420, 1829-2099, |
| | 1829-2241, 1886-2263, 1890-2337, 1895-2293, 1916-2484, 1925-2485, 1930-2485, 1973-2327, 1973-2344, 2013-2430, |
| | 2074-2295, 2100-2758, 2117-2513, 2146-2650, 2146-2763, 2158-2461, 2255-2804, 2257-2580, 2284-2548, |
| | 2287-2458, 2291-2742, 2297-2542, 2306-2575, 2367-2821, 2402-2658, 2444-2967, 2445-3007, 2452-3198, 2456-3174, |
| | 2485-3172, 2491-2782, 2492-2773, 2516-2778, 2521-3105, 2523-3091, 2548-2853, 2548-2864, 2569-2832, |
| | 2569-3101, 2571-3016, 2571-3137, 2576-3134, 2584-2854, 2632-2900, 2632-2901, 2644-2813, 2644-3235, 2644-3263, |
| | 2653-3176, 2653-3190, 2653-3256, 2665-3188, 2668-2934, 2689-3263, 2745-3214, 2781-3111, 2795-3209, |
| | 2798-3263, 2801-3263, 2808-3261, 2809-3181, 2852-3244, 2860-3237, 2947-3248, 2957-3246, 2960-3246, |
| | 2986-3246, 3000-3246, 3008-3232, 3058-3256 |

TABLE 5

| Polynucleotide SEQ | | |
|---|---|---|
| ID NO: | Incyte Project ID: | Representative Library |
| 26 | 2780338CB1 | THYMNOE02 |
| 27 | 2317440CB1 | BRACNOK02 |
| 28 | 3013470CB1 | MUSCNOT07 |
| 29 | 1738823CB1 | TESTNOT03 |
| 30 | 4184551CB1 | TESTNOT11 |
| 31 | 70042484CB1 | BRAUNOR01 |
| 32 | 3236274CB1 | PROSNOT28 |
| 33 | 7179725CB1 | LNODNOT03 |
| 34 | 1966217CB1 | LUNGNOT02 |
| 35 | 1598186CB1 | EOSITXT01 |
| 37 | 7925017CB1 | COLNTUS02 |
| 38 | 6758789CB1 | BRAENOK01 |
| 39 | 7488249CB1 | THYRNOT03 |
| 40 | 5046311CB1 | LIVRNON08 |
| 41 | 931056CB1 | TESTNOC01 |
| 42 | 2578937CB1 | NGANNOT01 |
| 43 | 489786CB1 | LIVRNON08 |
| 44 | 2240034CB1 | BMARTXE01 |
| 45 | 3438037CB1 | PITUNOT06 |
| 46 | 6578021CB1 | LATRTUT02 |
| 47 | 8013295CB1 | BRAIFER05 |
| 48 | 5001859CB1 | LUNGNON03 |
| 49 | 7506133CB1 | BRAITUT07 |
| 50 | 5301066CB1 | BRAINOT23 |

TABLE 6

| Library | Vector | Library Description |
|---|---|---|
| BMARTXE01 | pINCY | This 5′ biased random primed library was constructed using RNA isolated from treated SH-SY5Y cells derived |
| | | from a metastatic bone marrow neuroblastoma, removed from a 4-year-old Caucasian female (Schering AG). The |
| | | medium was MEM/HAM'S F12 with 10% fetal calf serum. After reaching about 80% confluency cells were treated |
| | | with 6-Hydroxydopamine (6-OHDA) at 100 microM for 8 hours. |
| BRACNOK02 | PSPORT1 | This amplified and normalized library was constructed using RNA isolated from posterior cingulate tissue |
| | | removed from an 85-year-old Caucasian female who died from myocardial infarction and retroperitoneal |
| | | hemorrhage. Pathology indicated atherosclerosis, moderate to severe, involving the circle of Willis, middle cerebral, |
| | | basilar and vertebral arteries; infarction, remote, left dentate nucleus; and amyloid plaque deposition consistent |
| | | with age. There was mild to moderate leptomeningeal fibrosis, especially over the convexity of the frontal lobe. |
| | | There was mild generalized atrophy involving all lobes. The white matter was mildly thinned. Cortical thickness in |
| | | the temporal lobes, both maximal and minimal, was slightly reduced. The substantia nigra pars compacta appeared |
| | | mildly depigmented. Patient history included COPD, hypertension, and recurrent deep venous thrombosis. |
| | | 6.4 million independent clones from this amplified library were normalized in one round using conditions adapted |
| | | from Soares et al., PNAS (1994) 91: 9228-9232 and Bonaldo et al., Genome Research 6 (1996): 791. |
| BRAENOK01 | PSPORT1 | This amplified and normalized library was constructed using RNA isolated from inferior parietal cortex |
| | | tissue removed from a 35-year-old Caucasian male who died from cardiac failure. Pathology indicated moderate |
| | | leptomeningeal fibrosis and multiple microinfarctions of the cerebral neocortex. There was evidence of shrunken |
| | | and slightly eosinophilic pyramidal neurons throughout the cerebral hemispheres. There were multiple small |
| | | microscopic areas of cavitation with surrounding gliosis scattered throughout the cerebral cortex. |
| | | Patient history included dilated cardiomyopathy, congestive heart failure, and cardiomegaly. Patient medications |
| | | included simethicone, Lasix, Digoxin, Colace, Zantac, captopril, and Vasotec. 1.08 million independent |
| | | clones from this amplified library were normalized in one round using conditions adapted from Soares |
| | | et al., PNAS (1994) 91: 9228-9232 and Bonaldo et al., Genome Research 6 (1996): 791, except that a significantly |
| | | longer (48 hours/round) reannealing hybridization was used. |
| BRAIFER05 | pINCY | Library was constructed using RNA isolated from brain tissue removed from a Caucasian male fetus who was |
| | | stillborn with a hypoplastic left heart at 23 weeks' gestation. |
| BRAINOT23 | pINCY | Library was constructed using RNA isolated from right temporal lobe tissue removed from a 45-year-old Black male |
| | | during a brain lobectomy. Pathology for the associated tumor tissue indicated dysembryoplastic neuroepithelial |
| | | tumor of the right temporal lobe. The right temporal region dura was consistent with calcifying pseudotumor of |
| | | the neuraxis. The patient presented with convulsive intractable epilepsy, partial epilepsy, and memory disturbance. |
| | | Patient history included obesity, meningitis, backache, unspecified sleep apnea, acute stressreaction, acquired knee |
| | | deformity, and chronic sinusitis. Family history included obesity, benign hypertension, cirrhosis of the liver, |
| | | alcohol abuse, hyperlipidemia, cerebrovascular disease, and type II diabetes. |
| BRAITUT07 | pINCY | Library was constructed using RNA isolated from left frontal lobe tumor tissue removed from the brain of a 32- |
| | | year-old Caucasian male during excision of a cerebral meningeal lesion. Pathology indicated low grade desmoplastic |
| | | neuronal neoplasm, type not otherwise specified. The lesion formed a firm, circumscribed cyst-associated mass |
| | | involving white matter and cortex. No definite glial component was evident to suggest a diagnosis of ganglioglioma. |
| | | Family history included atherosclerotic coronary artery disease. |
| BRAUNOR01 | pINCY | This random primed library was constructed using RNA isolated from striatum, globus pallidus and posterior |
| | | putamen tissue removed from an 81-year-old Caucasian female who died from a hemorrhage and ruptured thoracic |
| | | aorta due to atherosclerosis. Pathology indicated moderate atherosclerosis involving the internal carotids, bilaterally; |
| | | microscopic infarcts of the frontal cortex and hippocampus; and scattered diffuse amyloid plaques and |
| | | neurofibrillary tangles, consistent with age. Grossly, the leptomeninges showed only mild thickening and |
| | | hyalinization along the superior sagittal sinus. The remainder of the leptomeninges was thin and contained some |
| | | congested blood vessels. Mild atrophy was found mostly in the frontal poles and lobes, and temporal lobes, |
| | | bilaterally. Microscopically, there were pairs of Alzheimer type II astrocytes within the deep layers of the neocortex. |
| | | There was increased satellitosis around neurons in the deep gray matter in the middle frontal cortex. The amygdala |
| | | contained rare diffuse plaques and neurofibrillary tangles. The posterior hippocampus contained a microscopic area |
| | | of cystic cavitation with hemosiderin-laden macrophages surrounded by reactive gliosis. Patient history included |
| | | sepsis, cholangitis, post-operative atelectasis, pneumonia CAD, ardiomegaly due to left ventricular hypentrophy, |
| | | splenomegaly, arteriolonephrosclerosis, nodular colloidal goiter, emphysema, CHF, hypothyroidism, |
| | | and peripheral vascular disease. |
| COLNTUS02 | pINCY | This subtracted library was constructed using 1.16 million clones from a pooled colon tumor library and was |
| | | subjected to 2 rounds of subtraction hybridization with 7 million clones from a colon tissue library. The starting |
| | | library for subtraction was constructed using pooled cDNA from 6 donors. cDNA was generated using mRNA |
| | | isolated from colon tumor tissue removed from a 55-year-old Caucasian male (A) during hemicolectomy; from a 60- |
| | | year-old Caucasian male (B) during hemicolectomy; from a 62-year-old Caucasian male (C) during sigmoidectomy; |
| | | from a 30-year-old Caucasian female (D) during hemicolectomy; from a 64-year-old Caucasian female (E) during |
| | | hemicolectomy; and from a 70-year-old Caucasian female (F) during hemicolectomy. Pathology indicated invasive |
| | | grade 3 adenocarcinoma (A); invasive grade 2 adenocarcinoma (B); invasive grade 2 adenocarcinoma (C); |
| | | carcinoid tumor (D); invasive grade 3 adenocarcinoma (E); and invasive grade 2 adenocarcinoma (F). |
| | | Patient medications included Ativan (A); Seldane (B), Tri-Levlen (D); Synthroid (E); Tamoxifen, prednisone, |
| | | Synthroid, and Glipizide (F). The hybridization probe for subtraction was derived from a similarly constructed |
| | | library using RNA isolated from colon tissue from a different donor. Subtractive hybridization conditions were |
| | | based on the methodologies of Swaroop et al., NAR 19 (1991): 1954 and Bonaldo, et al., |
| | | Genome Research 6 (1996): 791. |
| EOSITXT01 | pINCY | Library was constructed using RNA isolated from eosinophils stimulated with IL-5. |
| LATRTUT02 | pINCY | Library was constructed using RNA isolated from a myxoma removed from the left atrium of a 43-year-old |
| | | Caucasian male during annuloplasty. Pathology indicated atrial myxoma. Patient history included pulmonary |
| | | insufficiency, acute myocardial infarction, atherosclerotic coronary artery disease, hyperlipidemia, and tobacco use. |
| | | Family history included benign hypertension, acute myocardial infarction, atherosclerotic coronary artery disease, |
| | | and type II diabetes. |
| LIVRNON08 | pINCY | This normalized library was constructed from 5.7 million independent clones from a pooled liver tissue library. |
| | | Starting RNA was made from pooled liver tissue removed from a 4-year-old Hispanic male who died from anoxia |
| | | and a 16 week female fetus who died after 16-weeks gestation from anencephaly. Serologies were positive for |
| | | cytolomegalovirus in the 4-year-old. Patient history included asthma in the 4-year-old. Family history included |
| | | taking daily prenatal vitamins and mitral valve prolapse in the mother of the fetus. The library was normalized |
| | | in 2 rounds using conditions adapted from Soares et al., PNAS (1994) 91: 9228 and Bonaldo et al., Genome |
| | | Research 6 (1996): 791, except that a significantly longer (48 hours/round) reannealing hybridization was used. |
| LNODNOT03 | pINCY | Library was constructed using RNA isolated from lymph node tissue obtained from a 67-year-old Caucasian male |
| | | during a segmental lung resection and bronchoscopy. On microscopic exam, this tissue was found to be extensively |
| | | necrotic with 10% viable tumor. Pathology for the associated tumor tissue indicated invasive grade 3-4 squamous |
| | | cell carcinoma. Patient history included hemangioma. Family history included atherosclerotic coronary artery |
| | | disease, benign hypertension, congestive heart failure, atherosclerotic coronary artery disease. |
| LUNGNON03 | PSPORT1 | This normalized library was constructed from 2.56 million independent clones from a lung tissue library. |
| | | RNA was made from lung tissue removed from the left lobe of a 58-year-old Caucasian male during a segmental |
| | | lung resection. Pathology for the associated tumor tissue indicated a metastatic grade 3 (of 4) osteosarcoma. |
| | | Patient history included soft tissue cancer, secondary cancer of the lung, prostate cancer, and an acute |
| | | duodenal ulcer with hemorrhage. Patient also received radiation therapy to the retroperitoneum. Family history |
| | | included prostate cancer, breast cancer, and acute leukemia. The normalization and hybridization conditions were |
| | | adapted from Soares et al., PNAS (1994) 91: 9228; Swaroop et al., NAR |
| | | (1991) 19: 1954; and Bonaldo et al., Genome Research (1996) 6: 791. |
| LUNGNOT02 | PBLUBSCRIPT | Library was constructed using RNA isolated from the lung tissue of a 47-year-old Caucasian male, who died of a |
| | | subarachnoid hemorrhage. |
| MUSCNOT07 | pINCY | Library was constructed using RNA isolated from muscle tissue removed from the forearm of a 38-year-old |
| | | Caucasian female during a soft tissue excision. Pathology for the associated tumor tissue indicated intramuscular |
| | | hemangioma. Family history included breast cancer, benign hypertension, cerebrovascular disease, |
| | | colon cancer, and type II diabetes. |
| NGANNOT01 | PSPORT1 | Library was constructed using RNA isolated from tumorous neuroganglion tissue removed from a 9-year-old |
| | | Caucasian male during a soft tissue excision of the chest wall. Pathology indicated a ganglioneuroma. Family history |
| | | included asthma. |
| PITUNOT06 | pINCY | Library was constructed using RNA isolated from pituitary gland tissue removed from a 55-year-old male who |
| | | died from chronic obstructive pulmonary disease. Neuropathology indicated there were no gross abnormalities, |
| | | other than mild ventricular enlargement. There was no apparent microscopic abnormality in any of the neocortical |
| | | areas examined, except for a number of silver positive neurons with apical dendrite staining, particularly in the |
| | | frontal lobe. The significance of this was undetermined. The only other microscopic abnormality was that there was |
| | | prominent silver staining with some swollen axons in the CA3 region of the anterior and posterior hippocampus. |
| | | Microscopic sections of the cerebellum revealed mild Bergmann's gliosis in the Purkinje cell layer. |
| | | Patient history included schizophrenia. |
| PROSNOT28 | pINCY | Library was constructed using RNA isolated from diseased prostate tissue removed from a 55-year-old |
| | | Caucasian male during a radical prostatectomy and regional lymph node excision. Pathology indicated |
| | | adenofibromatous hyperplasia. Pathology for the associated tumor tissue indicated adenocarcinoma, Gleason |
| | | grade 5 + 4. The patient presented with elevated prostate specific antigen (PSA). Family history included |
| | | lung and breast cancer. |
| TESTNOC01 | PBLUESCRIPT | This large size fractionated library was constructed using RNA isolated from testicular tissue removed from a |
| | | pool of eleven, 10 to 61-year-old Caucasian males. |
| TESTNOT03 | PBLUESCRIPT | Library was constructed using RNA isolated from testicular tissue removed from a 37-year-old Caucasian male, |
| | | who died from liver disease. Patient history included cirrhosis, jaundice, and liver failure. |
| TESTNOT11 | pINCY | Library was constructed using RNA isolated from testicular tissue removed from a 16-year-old Caucasian male |
| | | who died from hanging. Patient history included drug use (tobacco, marijuana, and cocaine use), and medications |
| | | included Lithium, Ritalin, and Paxil. |
| THYMNOE02 | PCDNA2.1 | This 5′ biased random primed library was constructed using RNA isolated from thymus tissue removed from a |
| | | 3-year-old Hispanic male during a thymectomy and closure of a patent ductus arteriosus. The patient presented |
| | | with severe pulmonary stenosis and cyanosis. Patient history included a cardiac catheterization and echocardiogram. |
| | | Previous surgeries included Blalock-Taussig shunt and pulmonary valvotomy. The patient was not taking any |
| | | medications. Family history included benign hypertension, osteoarthritis, depressive disorder, |
| | | and extrinsic asthma in the grandparent(s). |
| THYRNOT03 | pINCY | Library was constructed using RNA isolated from thyroid tissue removed from the left thyroid of a 28-year-old |
| | | Caucasian female during a complete thyroidectomy. Pathology indicated a small nodule of adenomatous hyperplasia |
| | | present in the left thyroid. Pathology for the associated tumor tissue indicated dominant follicular adenoma, |
| | | forming a well-encapsulated mass in the left thyroid. |

TABLE 7

| Program | Description | Reference | Parameter Threshold |
|---|---|---|---|
| ABI | A program that removes vector sequences and masks | Applied Biosystems, | |
| FACTURA | ambiguous bases in nucleic acid sequences. | Foster City, CA. | |
| ABI/ | A Fast Data Finder useful in | Applied Biosystems, | Mismatch < 50% |
| PARACEL | comparing and annotating amino | Foster City, CA; | |
| FDF | acid or nucleic acid sequences. | Paracel Inc., Pasadena, CA. | |
| ABI | A program that assembles nucleic acid sequences. | Applied Biosystems, | |
| AutoAssembler | | Foster City, CA. | |
| BLAST | A Basic Local Alignment Search Tool useful in | Altschul, S.F. et al. (1990) | ESTs: Probability |
| | sequence similarity search for amino acid and nucleic | J. Mol. Biol. 215: 403-410; | value = 1.0E−8 |
| | acid sequences. BLAST includes five functions: | Altschul, S.F. et al. (1997) | or less; |
| | blastp, blastn, blastx, tblastn, and tblastx. | Nucleic Acids Res. 25: 3389-3402. | Full Length sequences: |
| | | | Probability value = |
| | | | 1.0E−10 or less |
| FASTA | A Pearson and Lipman algorithm that searches for | Pearson, W. R. and | ESTs: fasta E |
| | similarity between a query sequence and a group of | D. J. Lipman (1988) Proc. Natl. | value = 1.06E−6; |
| | sequences of the same type. FASTA comprises as | Acad Sci. USA 85: 2444-2448; | Assembled ESTs: fasta |
| | least five functions: fasta, tfasta, fastx, tfastx, and | Pearson, W. R. (1990) Methods Enzymol. 183: 63-98; | Identity = 95% or |
| | ssearch. | and Smith, T. F. and M. S. Waterman (1981) | greater and |
| | | Adv. Appl. Math. 2: 482-489. | Match length = |
| | | | 200 bases or greater; |
| | | | fastx E value = |
| | | | 1.0E−8 or less; |
| | | | Full Length sequences: |
| | | | fastx score = |
| | | | 100 or greater |
| BLIMPS | A BLocks IMProved Searcher that matches a | Henikoff, S. and J. G. Henikoff (1991) | Probability value = |
| | sequence against those in BLOCKS, PRINTS, | Nucleic Acids Res. 19: 6565-6572; Henikoff, | 1.0E−3 or less |
| | DOMO, PRODOM, and PFAM databases to search | J. G. and S. Henikoff (1996) Methods | |
| | for gene families, sequence homology, and structural | Enzymol. 266: 88-105; and Attwood, T. K. et | |
| | fingerprint regions. | al. (1997) J. Chem. Inf. Comput. Sci. 37: 417-424. | |
| HMMER | An algorithm for searching a query sequence against | Krogh, A. et al. (1994) J. Mol. Biol. | PFAM, INCY, |
| | hidden Markov model (HMM)-based databases of | 235: 1501-1531; Sonnhammer, E. L. L. et al. | SMART, or |
| | protein family consensus sequences, such as PFAM, | (1988) Nucleic Acids Res. 26: 320-322; | TIGRFAM hits: |
| | INCY, SMART, AND TIGRFAM. | Durbin, R. et al. (1998) Our World View, in | Probability value = |
| | | a Nutshell, Cambridge Univ. Press, pp. 1-350. | 1.0E−3 or less |
| | | | Signal peptide hits: |
| | | | Score = 0 or greater |
| ProfileScan | An algorithm that searches for structural and | Gribskov, M. et al. (1988) CABIOS 4: 61-66; | Normalized quality |
| | sequence motifs in protein sequences that match | Gribskov, M. et al. (1989) Methods | score ≧ GCG |
| | sequence patterns defined in Prosite. | Enzymol. 183: 146-159; Bairoch, A. et al. | specified “HIGH” |
| | | (1997) Nucleic Acids Res. 25: 217-221. | value for that |
| | | | particular |
| | | | Prosite motif. |
| | | | Generally, score = |
| | | | 1.4-2.1. |
| Phred | A base-calling algorithm that examines automated | Ewing, B. et al. (1998) Genome Res. 8: 175-185; | |
| | sequencer traces with high sensitivity and probability. | Ewing, B. and P. Green (1998) Genome | |
| | | Res. 8: 186-194. | |
| Phrap | A Phils Revised Assembly Program including | Smith, T. F. and M. S. Waterman (1981) Adv. | Score = 120 or greater; |
| | SWAT and CrossMatch, programs based on efficient | Appl. Math. 2: 482-489; Smith, T. F. and | Match length = |
| | implementation of the Smith-Waterman algorithm, | M. S. Waterman (1981) J. Mol. Biol. 147: 195-197; | 56 or greater |
| | useful in searching sequence homology and | and Green, P., University of | |
| | assembling DNA sequences. | Washington, Seattle, WA. | |
| Consed | A graphical tool for viewing and editing Phrap | Gordon, D. et al. (1998) Genome Res. 8: 195-202. | |
| | assemblies. | | |
| SPScan | A weight matrix analysis program that scans protein | Nielson, H. et al. (1997) Protein Engineering | Score = 3.5 or greater |
| | sequences for the presence of secretory signal | 10: 1-6; Claverie, J. M. and S. Audic (1997) | |
| | peptides. | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos | |
| | determine orientation. | (1996) Protein Sci. 5: 363-371. | |
| TMHMMER | A program that uses a hidden Markov model (HMM) | Sonnhammer, E.L. et al. (1998) Proc. Sixth | |
| | to delineate transmembrane segments on protein | Intl. Conf. on Intelligent Systems for Mol. | |
| | sequences and determine orientation. | Biol., Glasgow et al., eds., The Am. Assoc. | |
| | | for Artificial Intelligence (AAAI) Press, | |
| | | Menlo Park, CA, | |
| | | and MIT Press, Cambridge, | |
| | | MA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

1. An isolated polypeptide selected from the group consisting of:
a) a poypeptide comprising an amino acid sequence selected from the group consisting of SEQ ID NO:1-25,
b) a polypeptide comprising a naturally occurring amino acid sequence at least 90% identical to an amino acid sequence selected from the group consisting of SEQ ID NO:1-5, SEQ ID NO:7-13, SEQ ID NO:15-16, SEQ ID NO:18-19, and SEQ ID NO:21-25,
c) a polypeptide comprising a naturally occurring amino acid sequence at least 96% identical to the amino acid sequence of SEQ ID NO:6,
d) a polypeptide comprising a naturally occurring amino acid sequence at least 98% identical to the amino acid sequence of SEQ ID NO:14,
e) a polypeptide comprising a naturally occurring amino acid sequence at least 94% identical to an amino acid sequence selected from the group consisting of SEQ ID NO:17 and SEQ ID NO:20,
f) a biological active fragment of a polypeptide having an amino acid sequence selected from the group consisting of SEQ ID NO:1-25, and
g) an immonogenic fragment of a polypeptide having an amino acid sequence selected from the group consisting of SEQ ID NO:1-25.

2. An isolated polypeptide of claim 1 comprising an amino acid sequence selected from the group consisting of SEQ ID NO:1-25.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:26-50.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-25.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:26-50,
b) a polynucleotide comprising a naturally occurring polynucleotide sequence at least 90% identical to a polynucleotide sequence selected from the group consisting of SEQ ID NO:26-41 and SEQ ID NO:43-50,
c) a polynucleotide comprising a naturally occurring polynucleotide sequence at least 95% identical to the polynucleotide sequence of SEQ ID NO:42,
d) a polynucleotide complementary to a polynucleotide of a),
e) a polynucleotide complementary to a polynucleotide of b),
f) a polynucleotide complementary to a polynucleotide of c), and
g) an RNA equivalent of a)-f).

13. (CANCELED).

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (CANCELED).

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-25.

19. (CANCELED).

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (CANCELED).

22. (CANCELED).

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (CANCELED).

25. (CANCELED).

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. (CANCELED).

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-105. (CANCELED).
